SEQUENCE LISTING (56 sequences)

SEQ ID NO: 1 - protein, 318 aa, Homo sapiens
MQRANHSTVTQFILVGFSVFPHLQLMLFLLFLLMYLFTLLGNLLIMATVWSERSLHTPMYLFLCALSVSEILYTVAIIPRMLADLLSTQRSIAFLACASQMFFSFSFGFTHSFLLTVMGYDRYVAICHPLRYNVLMSPRGCACLVGCSWAGGLVMGMVVTSAIFHLAFCGHKEIHHFACHVPPLLKLACGDDVLVVAKGVGLVCITALLGCFLLILLSYAFIVAAILKIPSAEGRNKAFSTCASHLTVVVVHYGFASVIYLKPKSPQSLEGDTLMGITYTVLTPFLSPIIFSLRNKELKVAMKKTFFSKLYPEKNVMM

SEQ ID NO: 2 - protein, 318 aa, Homo sapiens
MQRANHSTVTQFILVRFSVFPHLQLMLFLLFLLMYLFTLLGNLLIMATVWSERSLHTPMYLFLCALSVSEILYTVAIIPRMLADLLSTQRSIAFLACASQMFFSFSFGFTHSFLLTVMGYDRYVAICHPLRYNVLMSPRGCACLVGCSWAGGLVMGMVVTSAIFHLAFCGHKEIHHFACHVPPLLKLACGDDVLVVAKGVGLVCITALLGCFLLILLSYAFIVAAILKIPSAEGRNKAFSTCASHLTVVVVHYGFASVIYLKPKSPQSLEGDTLMGITYTVLTPFLSPIIFSLRNKELKVAMKKTFFSKLYPEKNVMM

SEQ ID NO: 3 - protein, 318 aa, Homo sapiens
MQRANHSTVTQFILVRFSVFPHLQLMLFLLFLLMYLFTLLGNLLIMATVWSERSLHTPMYLFLCVLSVSEILYTVAIIPRMLADLLSTQRSIAFLACASQMFFSFSFGFTHSFLLTVMGYDRYVAICHPLRYNVLMSPRGCACLVGCSWAGGLVMGMVVTSAIFHLAFCGHKEIHHFACHVPPLLKLACGDDVLVVAKGVGLVCITALLGCFLLILLSYAFIVAAILKIPSAEGRNKAFSTCASHLTVVVVHYGFASVIYLKPKSPQSLEGDTLMGITYTVLTPFLSPIIFSLRNKELKVAMKKTFFSKLYPEKNVMM

SEQ ID NO: 4 - protein, 318 aa, Homo sapiens
MQRANHSTVTQFILVGFSVFPHLQLMLFLLFLLMYLFTLLGNLLIMATVWSERSLHTPMYLFLCALSVSEILYTVAIIPRMLADLLSTQRSIAFLACASQMFFSFSFGFTHSFLLTVMGYDRYVAICHPLRYNVLMSPRGCACLVGCSWAGGLVMGMVVTSAIFHLAFCGHKEIQHFACHVPPLLKLACGDDVLVVAKGVGLVCITALLGCFLLILLSYAFIVAAILKIPSAEGRNKAFSTCASHLTVVVVHYGFASVIYLKPKSPQSLEGDTLMGITYTVLTPFLSPIIFSLRNKELKVAMKKTFFSKLYPEKNVMM

SEQ ID NO: 5 - protein, 25 aa, Homo sapiens
MQRANHSTVTQFILVGFSVFPHLQL

SEQ ID NO: 6 - protein, 24 aa, Homo sapiens
AIIPRMLADLLSTQRSIAFLACAS

SEQ ID NO: 7 - protein, 37 aa, Homo sapiens
SAIFHLAFCGHKEIHHFACHVPPLLKLACGDDVLVVA

SEQ ID NO: 8 - protein, 13 aa, Homo sapiens
YLKPKSPQSLEGD

SEQ ID NO: 9 - protein, 25 aa, Homo sapiens
MQRANHSTVTQFILVRFSVFPHLQL

SEQ ID NO: 10 - protein, 37 aa, Homo sapiens
SAIFHLAFCGHKEIQHFACHVPPLLKLACGDDVLVVA

SEQ ID NO: 11 - protein, 317 aa, Macaca fascicularis
MQRANHSTVTQFILVGFSAFPDLQLMLFLLFLLMYLFTLLGNLLIMATVWSERSLHTPMYLFLCALSVSEILYTVAIIPRMLADLLSTQRSIAFLACASQMFFSFSFGFTHSFLLTVMGYDRYVAICRPLRYNVLMSPRGGACLVGCSWAGGSVMGLVVTTAIFHLTFCGPSEIHHFACHVPPLLKLACGNNVPAVALGVGLVCITALLGCFLLILLSYAFIVAAILKIPSAEGRNKAFSTCASHVTVVVVHYGFASVIYLKPKGPQSLEGDTLMGITYTVLTPFLSPIIFSLRNKELKVAMKTFFSKLYPEKNVTI

SEQ ID NO: 12 - protein, 318 aa, Homo sapiens
MQRANHSTVTQFILVGFSVFPHLQLMLFLLFLLMYLFTLLGNLLIMATVWSERSLHTPMYLFLCALSVSEILYTVAIIPRMLADLLSTQRSIAFLACASQMFFSFSFGFTHSFLLTVMGYDRYVAICHPLRYNVLMSPRGCACLVGCSWAGGLVMGMVVTSAIFHLTFCGHKEIHHFACHVPPLLKLACGDDVLVVAKGVGLVCITALLGCFLLILLSYAFIVAAILKIPSAEGRNKAFSTCASHLTVVVVHYGFASVIYLKPKSPQSLEGDTLMGITYTVLTPFLSPIIFSLRNKELKVAMKKTFFSKLYPEKNVMM

SEQ ID NO: 13 - protein, 37 aa, Homo sapiens
SAIFHLTFCGHKEIHHFACHVPPLLKLACGDDVLVVA

SEQ ID NO: 14 - protein, 315 aa, Homo sapiens
MQGLNHTSVSEFILVGFSAFPHLQLMLFLLFLLMYLFTLLGNLLIMATVWSERSLHMPMYLFLCALSITEILYTVAIIPRMLADLLSTQRSIAFLACASQMFFSFSFGFTHSFLLTVMGYDRYVAICHPLRYNVLMSLRGCTCRVGCSWAGGLVMGMVVTSAIFHLAFCGHKEIHHFFCHVPPLLKLACGDDVLVVAKGVGLVCITALLGCFLLILLSYAFIVAAILKIPSAEGRNKAFSTCASHLTVVVVHYGFASVIYLKPKGPQSPEGDTLMGITYTVLTPFLSPIIFSLRNKELKVAMKKTCFTKLFPQNC

SEQ ID NO: 15 - protein, 315 aa, Homo sapiens
MLGLNHTSMSEFILVGFSAFPHLQLMLFLLFLLMYLFTLLGNLLIMATVWSERSLHTPMYLFLCVLSVSEILYTVAIIPRMLADLLSTQRSIAFLACASQMFFSFSFGFTHSFLLTVMGYDRYVAICHPLRYNVLMSPRGCACLVGCSWAGGSVMGMVVTSAIFQLTFCGSHEIQHFLCHVPPLLKLACGNNVPAVALGVGLVCIMALLGCFLLILLSYAFIVADILKIPSAEGRNKAFSTCASHLIVVIVHYGFASVIYLKPKGPHSQEGDTLMATTYAVLTPFLSPIIFSLRNKELKVAMKRTFLSTLYSSGT

SEQ ID NO: 16 - protein, 25 aa, Homo sapiens
MQGLNHTSVSEFILVGFSAFPHLQL

SEQ ID NO: 17 - protein, 25 aa, Homo sapiens
MLGLNHTSMSEFILVGFSAFPHLQL

SEQ ID NO: 18 - protein, 24 aa, Homo sapiens
AIIPRMLADLLSTQRSIAFLACAS

SEQ ID NO: 19 - protein, 24 aa, Homo sapiens
AIIPRMLADLLSTQRSIAFLACAS

SEQ ID NO: 20 - protein, 37 aa, Homo sapiens
SAIFHLAFCGHKEIHHFFCHVPPLLKLACGDDVLVVA

SEQ ID NO: 21 - protein, 37 aa, Homo sapiens
SAIFQLTFCGSHEIQHFLCHVPPLLKLACGNNVPAVA

SEQ ID NO: 22 - protein, 13 aa, Homo sapiens
YLKPKGPQSPEGD

SEQ ID NO: 23 - protein, 13 aa, Homo sapiens
YLKPKGPHSQEGD

SEQ ID NO: 24 - RNA, 19 nt, Homo sapiens
ggagacaccuugaugggca

SEQ ID NO: 25 - RNA, 19 nt, Homo sapiens
aguaaacucuacccagaaa

SEQ ID NO: 26 - RNA, 19 nt, Homo sapiens
gcagagagccaaucacucc

SEQ ID NO: 27 - RNA, 19 nt, Homo sapiens
ggucgugcacuauggcuuu

SEQ ID NO: 28 - RNA, 21 nt, Homo sapiens
gttcctgctgatgtacctgtt

SEQ ID NO: 29 - RNA, 21 nt, Homo sapiens
tgcgctacaacgtgctcatga

SEQ ID NO: 30 - RNA, 21 nt, Homo sapiens
tggctttgcctccgtcattta

SEQ ID NO: 31 - RNA, 21 nt, Homo sapiens
tctgctgaaggtcggaacaag

SEQ ID NO: 32 - RNA, 21 nt, Homo sapiens
acacaaggagatccaccattt

SEQ ID NO: 33 - protein, 7 aa, Rattus norvegicus
  1C3-A1-A1 heavy-chain CDR1 amino acid sequence
FTFDNAW

SEQ ID NO: 34 - protein, 10 aa, Rattus norvegicus
  1C3-A1-A1 heavy-chain CDR2 amino acid sequence
IKAKSNNYAT

SEQ ID NO: 35 - protein, 6 aa, Rattus norvegicus
  1C3-A1-A1 heavy-chain CDR3 amino acid sequence
TRLYYD

SEQ ID NO: 36 - protein, 115 aa, Rattus norvegicus
  1C3-A1-A1 heavy-chain variable domain amino acid sequence
EVQLVETGGNLVQPGKSLKLTCATSGFTFDNAWMHWVRQSPEKQLEWVAQIKAKSNNYATYYAESVKGRFTISRDDSKSSVYLQMNRLKEEDTAIYYCTRLYYDWGQGVMVTVSS

SEQ ID NO: 37 - protein, 6 aa, Rattus norvegicus
  1C3-A1-A1 light-chain CDR1 amino acid sequence
QDIGNY

SEQ ID NO: 38 - protein, 4 aa, Rattus norvegicus
  1C3-A1-A1 light-chain CDR2 amino acid sequence
  SITE 4: Xaa can be any amino acid
RATX

SEQ ID NO: 39 - protein, 8 aa, Rattus norvegicus
  1C3-A1-A1 light-chain CDR3 amino acid sequence
LQHKQYPL

SEQ ID NO: 40 - protein, 108 aa, Rattus norvegicus
  1C3-A1-A1 light-chain variable domain amino acid sequence
DIQMTQSPSSISVSLGDRFTFTCRASQDIGNYLSWFQQKPEKSPKLMIYRATNLEDGVPSRFSGSRSGSDYSLTINSLESEDTGIYFCLQHKQYPLTFGSGTKLEIKR

SEQ ID NO: 41 - protein, 7 aa, Rattus norvegicus
  1C3-A1-A2 heavy-chain CDR1 amino acid sequence
FTFSNYD

SEQ ID NO: 42 - protein, 10 aa, Rattus norvegicus
  1C3-A1-A2 heavy-chain CDR2 amino acid sequence
ISHSGDSTYF

SEQ ID NO: 43 - protein, 6 aa, Rattus norvegicus
  1C3-A1-A2 heavy-chain CDR3 amino acid sequence
HGVYTN

SEQ ID NO: 44 - protein, 122 aa, Rattus norvegicus
  1C3-A1-A2 heavy-chain variable domain amino acid sequence
EVQLQESGGGLIQPGRSLKLSCAASGFTFSNYDMAWVRQAPTKGLEWVASISHSGDSTYFRASVKGRFTVSRDNAKSSLYLQMDSLRSEDTATYYCARHGVYTNYGIWFAYWGQGTLVTVSS

SEQ ID NO: 45 - protein, 6 aa, Rattus norvegicus
  1C3-A1-A2 light-chain CDR1 amino acid sequence
QDIGNY

SEQ ID NO: 46 - protein, 4 aa, Rattus norvegicus
  1C3-A1-A2 light-chain CDR2 amino acid sequence
  SITE 4: Xaa can be any amino acid
RATX

SEQ ID NO: 47 - protein, 8 aa, Rattus norvegicus
  1C3-A1-A2 light-chain CDR3 amino acid sequence
LQHKQYPL

SEQ ID NO: 48 - protein, 108 aa, Rattus norvegicus; identical to SEQ ID NO: 40
  1C3-A1-A2 light-chain variable domain amino acid sequence

SEQ ID NO: 49 - protein, 7 aa, Rattus norvegicus
  8A11-B9-A1 heavy-chain CDR1 amino acid sequence
FTFSSAW

SEQ ID NO: 50 - protein, 10 aa, Rattus norvegicus
  8A11-B9-A1 heavy-chain CDR2 amino acid sequence
IKGKSNNYAT

SEQ ID NO: 51 - protein, 8 aa, Rattus norvegicus
  8A11-B9-A1 heavy-chain CDR3 amino acid sequence
TWFGPMDA

SEQ ID NO: 52 - protein, 117 aa, Rattus norvegicus
  8A11-B9-A1 heavy-chain variable domain amino acid sequence
EVQLQESGGRLVQPGKSLKLTCAASGFTFSSAWIHWVRQSPEKQLEWVAQIKGKSNNYATYYAESVKGRFTISRDDSKSSVYLQMNSLKEEDTAIYYCTWFGPMDAWGQGASVTVSS

SEQ ID NO: 53 - protein, 11 aa, Rattus norvegicus
  8A11-B9-A1 light-chain CDR1 amino acid sequence
KSLLHNNGKTF

SEQ ID NO: 54 - protein, 4 aa, Rattus norvegicus
  8A11-B9-A1 light-chain CDR2 amino acid sequence
  SITE 4: Xaa can be any amino acid
WMSX

SEQ ID NO: 55 - protein, 8 aa, Rattus norvegicus
  8A11-B9-A1 light-chain CDR3 amino acid sequence
QQFLEYPL

SEQ ID NO: 56 - protein, 113 aa, Rattus norvegicus
  8A11-B9-A1 light-chain variable domain amino acid sequence
DIVMTQGALPNPVPSGESASITCQSSKSLLHNNGKTFLNWYLQRPGQSPQLLIYWMSTRASGVSDRFSGSGSGADFTLKISSVEAEDVGVYYCQQFLEYPLTFGSGTKLEIKR

(19) United States
(12) Patent Application Publication
(10) Pub. No.: US 2020/0024347 A1
(43) Pub. Date: Jan. 23, 2020

(54) OR10H1 ANTIGEN BINDING PROTEINS AND USES THEREOF

(71) Applicant: iOmx Therapeutics AG, Martinsried (DE)

(72) Inventors: Sebastian MEIER-EWERT, Martinsried (DE); Nisit KHANDELWAL, Martinsried (DE); Philipp BECKHOVE, Regensburg (DE); Tillmann MICHELS, Regensburg (DE); Michael BOUTROS, Heidelberg (DE); Marco BREINIG, Schriesheim (DE); Antonio SORRENTINO, Heidelberg (DE); Valentina VOLPIN, Regensburg (DE)

(21) Appl. No.: 16/348,336

(22) PCT Filed: Nov. 10, 2017
(86) PCT No.: PCT/EP2017/078883

§ 371 Date: May 8, 2019

Related U.S. Application Data
(60) Provisional application No. 62/420,015, filed on Nov. 10, 2016.

(30) Foreign Application Priority Data
Nov. 10, 2016 (EP) 16198189.9
Nov. 10, 2016 (EP) PCT/EP2016/077288

(51) Int. Cl. C07K 16/28; C07K 14/705; G01N 33/574; G01N 33/563

(52) U.S. Cl. CPC C07K 16/286; C07K 14/70571; C07K 2317/34; G01N 33/563; C07K 2317/565; G01N 33/57484

(57) ABSTRACT

The invention provides antigen binding proteins that bind to one or more extracellular domains of the human olfactory receptor 10H1 (OR10H1) or variants thereof, including antigen binding proteins which can modulate the expression, function, activity and/or stability of such receptor, as well as providing nucleic acids encoding such antigen binding proteins or components thereof. Methods for detecting OR10H1 or variants thereof in a sample, including diagnostic methods, using such antigen binding proteins are also provided. Uses of such antigen binding proteins, and nucleic acids encoding the same, in medicine are further provided.

The invention provides antigen binding proteins that bind to one or more extracellular domains of the human olfactory receptor 10H1 (OR10H1) or variants thereof, including antigen binding proteins which can modulate the expression, function, activity and/or stability of such receptor, as well as providing nucleic acids encoding such antigen binding proteins or components thereof. Methods for detecting OR10H1 or variants thereof in a sample, including diagnostic methods, using such antigen binding proteins are also provided. Uses of such antigen binding proteins, and nucleic acids encoding the same, in medicine are further provided.

Therapies targeting or utilising the immune system, and therapeutic monoclonal antibodies (mAbs) in particular, have become an important treatment strategy in cancer and autoimmune disease. Numerous mAbs have been approved by the United States Food and Drug Administration (FDA) and the European Medicines Agency (EMA), with many more undergoing clinical evaluation and pre-clinical development.

In cancer, for example, there are a number of targets against which mAbs may be directed in order to eliminate tumour cells. mAbs can directly target molecules expressed on the tumour (e.g. CD20 on lymphoma) in order to elicit Fc-dependent cytotoxic effects. They can also interact with Fc receptor-expressing cells of the innate immune effector system to trigger antibody-dependent cell mediated cytotoxicity (ADCC), or react with serum or other proteins in order to elicit complement dependent cytotoxicity (CDC). Further, they can function by blocking key survival signals in the tumour cell, thereby provoking cell cycle arrest and cell death in addition to ADCC. Current state of the art cancer immunotherapies—involving antigen-specific vaccines or adoptive cellular therapies with tumour-specific cytotoxic T cells (CTLs)—have meanwhile developed protocols to induce functionally potent cellular CTL responses in patients (Gao et al, 2013).

However, one of the limitations associated with the use of mAbs in cancer is that tumour cells often exploit immune-checkpoints to prevent immune-recognition or downregulate the CTL response, thereby imposing resistance against immunotherapy (Rabinovich et al, 2007, and Zitvogel et al, 2006). Under normal conditions, such regulatory checkpoints are crucial for the maintenance of self-tolerance under physiological conditions but there is an increasing recognition of the important role that they play in cancer (Hanahan 2011). Cancer cells can take over these mechanisms or release immunosuppressive cytokines such as transforming growth factor β (TGF-β) to evade and suppress the immune system in order to develop into a tumour (Drake 2006). This results in a significant number of patients not sufficiently responding to T cell-based immunotherapy. Nevertheless, tumour infiltrating lymphocytes (TILs) that fail to eliminate the tumour but yet possess cytolytic functions in vitro suggest an endogenous anti-tumour effect that can be harnessed if immune tolerance in the tumour cell can be broken.

Blocking antibodies against surface-expressed immune-regulatory proteins is one such approach for breaking immune tolerance.

Ligation of the T-cell activator CD28 by a member of the B7 family of co-stimulatory molecules, B7-1 (also known as CD80) or B7-2 (also known as CD86), greatly enhances TCR-induced survival, proliferation, differentiation and eventual activation of the naive T-cell. Concurrently, cytotoxic T-lymphocyte associated antigen 4 (CTLA4) is transported from intracellular stores to the cell surface where it serves to down-regulate the T-cell activation signal by competitive binding to B7 molecules. For this reason, CTLA4 has been referred to as a brake on the immune system and was proposed as a viable target for blocking with mAbs to enhance anti-cancer responses. Indeed, blocking antibodies against CTLA4 have been shown to boost anti-tumour immunity (Chambers et al, 2001).

The programmed death 1 (PD-1) receptor expressed by activated T-cells is another key immune checkpoint receptor with a negative regulatory role when engaged by its ligands PD-L1 (also known as B7-H1) and PD-L2 (also known as B7-DC) within the tumour microenvironment. Indeed, blocking antibodies against PD-L1 have also been shown to boost anti-tumour immunity (Blank et al, 2004). Blocking antibodies against CTLA4 and PD-L1 have been successfully applied in clinical trials (van Elsas et al, 1999; Weber, 2007; Brahmer et al, 2012; Topalian et al, 2012).

Another checkpoint receptor identified to be a potential therapeutic target is V-domain Ig suppressor of T-cell activation (VISTA). This protein bears a structural resemblance to PD-1. Its expression on antigen presenting cells (APCs) inhibits T-cell proliferation and function. Application of an antagonistic mAb reversed T-cell inhibition and exacerbated a mouse model of autoimmunity, indicating that it is a checkpoint blocker amenable to therapeutic mAb manipulation (Wang et al, 2011).

Still, treatment unresponsiveness is frequent among patients (Topalian et al, 2012), indicating that other immune-checkpoint pathways may be active. Indeed, synergistic cooperation between several immune-inhibitory pathways maintains immune tolerance against tumours, which might explain why blocking only one immune-checkpoint node can still result in tumour escape (Berrien-Elliott et al, 2013, and Woo et al, 2012). However, little is known about the molecular factors playing an important role in such immune-inhibitory pathways.

Therefore, successful cancer immunotherapy requires a systematic delineation of the entire immune-regulatory circuit—the ‘immune modulatome’—expressed on tumours (Woo et al, 2012; Berrien-Elliott et al, 2013).

WO 2015/028515 describes a robust high-throughput RNAi assay that enables a qualitative as well as a quantitative analysis of heterologous cell-to-cell interaction between tumour cells and T cells. CTL-induced tumour cell death was used as the selection criterion for the screen. By systematically knocking down the expression of cell-surface expressed proteins in tumour cells and then assaying for CTL induced tumour cell death, the authors identified certain sensory receptors as potential modulators of the CTL response against tumour cells.

The publication of Khandelwal et al, 2015 described the same assay as that disclosed in WO 2015/028515, and additionally the validation of another target, C—C chemokine receptor type 9 (CCR9). CCR9 was found to regulate STAT signaling in T cells, resulting in reduced T-helper-1 cytokine secretion and reduced cytotoxic capacity.

Thus, to date, only a few immune-checkpoints which modulate the CTL response have been identified. Indeed, only a limited number of specific olfactory receptors have been described as being associated with such function: for example TAS2R3, OR1F1, OR2J2 and OR51E2 (WO 2015/028515). Therefore, today, there is still an unmet need for identifying further molecular targets that may serve as immune-checkpoints and in particular an unmet need for moieties, items, components, detectors and/or (other) means and methods to modulate, detect and otherwise utilise such possible immune-checkpoint targets, such as in medicine, diagnosis and research.

Accordingly, it is an object of the present invention to provide alternative, improved, simpler, cheaper and/or integrated moieties, items, components, detectors and/or (other) means or methods that address one or more of these or other problems. Such an object underlying the present invention is solved by the subject matter as disclosed or defined anywhere herein, for example by the subject matter of the attached claims.

Surprisingly, human OR10H1 is identified as a molecular target that plays an important role in the CTL response. OR10H1 is a G-protein coupled receptor (GPCR), predicted to have four extracellular domains. Human OR10H1 is 318 amino acids in length and arises from a single coding-exon gene located on chromosome 19 (RefSeqs: NP_039228.1; NM_013940.2; XP_011526213.1; XM_011527911.1; HUGO Gene Nomenclature Committee symbol: HGNC:8172; Genome Coordinates for assembly GRCh38.p7: Chr19:15,807,003-15,808,126; UniProtKB identifier Q9Y4A, Sequence version 1 of 1 Nov. 1999, Entry version 126 of 5 Oct. 2016). According to the Human Olfactory Data Explorer (HORDE) (https://genome.weizmann.ac.il/horde) and UniProt (http://www.uniprot.org), human OR10H1 has at least five naturally occurring variants: G16R (in the first extracellular domain; 13.9% frequency according to HORDE), A65V (transmembrane; A65V together with G16R has a 1.5% frequency according to HORDE), A142T (transmembrane; 0.5% frequency according to HORDE), A167T (in the third extracellular domain; 1.7% frequency according to HORDE), and H175Q (in the third extracellular domain).

Based on its homology to other olfactory receptors, OR10H1 is assumed to sense the chemical environment and can be distinguishable from other olfactory receptors by the chemostimulus/chemostimuli to which it responds. Olfactory receptors signal mainly via a unique G protein-coupled adenylyl cyclase cascade. Subsequently, cAMP is the key messenger of olfactory G protein signaling. Olfactory signaling leads to the specific cAMP production by adenylyl cyclase type III (AC3). AC3 in turn is activated by the olfactory-restricted G protein alpha subunit G-alpha-Olf. It has been shown that olfactory receptors could couple in vitro to G-alpha-s and G-alpha-15 G proteins, which might alter the specificity of the receptor. Furthermore, olfactory receptors can signal via other mechanisms. Olfactory receptor activation leads to production of cGMP, opens cyclic nucleotide-gated channels (CNC) by cAMP and cGMP, stimulates the production of Inositol-1,4,5-trisphosphate (IP3) and increases influx of calcium.

OR10H1 expression on tumour cells is found to inhibit T cell activity, reduce TIL type I cytokine secretion and induce apoptosis in T cells. In this manner, OR10H1 down-regulates the CTL response. Thus, the antigen binding proteins described herein by the present inventors that bind to one or more epitope(s) displayed by one or more extracellular domains of OR10H1 can be expected to solve one or more of the foregoing problems, such as to be useful in medicine, diagnosis or research. In particular, such antigen binding proteins that inhibit or antagonise the expression, function, activity and/or stability of OR10H1 or variants thereof are expected to be useful in the treatment, diagnosis and/or prevention of disorders characterized by resistance to T cell-mediated cytotoxicity, such as cancer.

Generally, and by way of brief description, the main aspects of the present invention can be described as follows:

The present invention inter alia provides novel antigen binding proteins (ABPs) that bind to human OR10H1, or a paralogue, orthologue or other variant thereof.

Accordingly, in a first aspect, the present invention provides an ABP that binds to human OR10H1, or a paralogue, orthologue or other variant thereof, wherein said ABP binds to one or more epitope(s) displayed by one or more extracellular domain(s) of said OR10H1, paralogue, orthologue or other variant.

In a second aspect, the present invention provides a nucleic acid encoding an ABP of the invention or a component of said ABP.

In a third aspect, the present invention provides a nucleic acid construct (NAC) comprising a nucleic acid encoding an ABP of the invention or a component of said ABP, and one or more additional features permitting the expression of the encoded ABP or component of said ABP in a cell.

In a fourth aspect, the present invention provides a cell comprising a NAC of the invention capable of expressing the encoded ABP or component of said ABP.

In a fifth aspect, the present invention provides a pharmaceutical composition comprising the ABP of the invention or a component of said ABP, and/or at least one NAC encoding the ABP of the invention or a component of said ABP, and/or cells of the invention, and a pharmaceutically acceptable excipient or carrier.

In a sixth aspect, the present invention provides a method of modulating the expression, function, activity and/or stability of a human OR10H1 or paralogue, orthologue or other variant thereof comprising contacting a cell that expresses an OR10H1 or variant with an ABP of the invention, or an NAC encoding said ABP, thereby modulating the expression, function, activity and/or stability of said OR10H1 or variant.

In a seventh aspect, the present invention provides a method of enhancing a cell-mediated immune response to a mammalian cell, such as a human cell, that expresses a human OR10H1 or paralogue, orthologue or other variant thereof, comprising contacting said cell with an ABP of the invention, or an NAC encoding said ABP, in the presence of an immune cell, such as a T-cell, wherein said ABP is an inhibitor of the expression, function, activity and/or stability of said OR10H1 or variant, thereby enhancing a cell-mediated immune response.

In an eighth aspect, the present invention provides the ABP, the NAC, the cells or the pharmaceutical composition of the invention for use in medicine, such as in the treatment or the prevention of a disease, disorder or condition in a mammalian subject, such as a human patient.

In a ninth aspect, the present invention provides a method of treating or preventing a disease, disorder or condition in a mammalian subject in need thereof, comprising administering to said subject at least once an effective amount of the ABP, the NAC, the cells or the pharmaceutical composition of the invention.

In a tenth aspect, the present invention provides a method of detecting a human OR10H1 or a paralogue, orthologue or other variant thereof in a sample, comprising contacting the sample with an ABP of the invention and detecting binding between said ABP and said OR10H1, paralogue, orthologue or other variant.

In an eleventh aspect, the present invention provides a method of determining whether a mammalian subject, such as a human, has or is at risk of developing a phenotype associated with cellular resistance against a cell-mediated immune response, such as a T-cell-mediated immune response, comprising:
(a) the steps of:
(i) providing a biological sample from said subject, said sample comprising cells or tissue of said subject, or an extract of said cells or tissue; and
(ii) contacting the sample with an ABP of the invention and detecting binding between said ABP and a human OR10H1 or a paralogue, orthologue or other variant thereof;
or
(b) contacting a biological sample from said subject, said sample comprising cells or tissue of said subject, or an extract of said cells or tissue, with an ABP of the invention and detecting binding between said ABP and a human OR10H1 or a paralogue, orthologue or other variant thereof in said sample:

wherein the detection of a human OR10H1 or paralogue, orthologue or other variant thereof in said sample indicates a phenotype associated with cellular resistance against the cell-mediated immune response in said subject.

In a twelfth aspect, the present invention provides a kit for detecting human OR10H1 or a paralogue, orthologue or other variant thereof in a biological sample comprising:
(i) at least one ABP of the invention, and
(ii) optionally, instructions for, and/or one or more additional components suitable for, detecting the binding between said ABP and said OR10H1 or variant.

In a thirtheenth aspect, the present invention provides a cell, which is either: (i) a hybridoma capable of expressing an ABP of the invention; or (ii) a cell comprising at least one NAC of the invention encoding an ABP or a component of said ABP of the invention.

In a fourteenth aspect, the present invention provides a method of producing an ABP of the invention, comprising culturing one or more cells of the invention under conditions allowing the expression of said ABP.

In a fifteenth aspect, the present invention provides a method of manufacturing a pharmaceutical composition of the invention, comprising formulating the ABP isolated by the method of producing an ABP according to the invention into a pharmaceutically acceptable form.

In a sixteenth aspect, the present invention provides a method for identifying (or characterising) a compound suitable for the treatment of a disease characterized by expression (such by aberrant expression) of OR10H1 or a paralogue, orthologue or other variant thereof.

### BRIEF DESCRIPTION OF THE DRAWINGS

FIG. 1AA: OR10H1 is expressed by various cancer cell-lines. RT-PCR shows expression of OR10H1 by the cell lines M579-A2 (melanoma), PANG-1 (PDAC) and SW480 (colorectal), but not by the myeloma cell line KMM-1.

FIG. 1 cont.: OR10H1 prevents lysis of solid tumors by TILs. AB M579-A2 cells were transfected with the siRNA sequences for OR10H1 and mRNA was measured by RT-PCR after 72 h. Beta-actin served as a control. B, C Killing assays of M579-A2 with two different TIL-cultures. B M579-A2-luc cells, which were transfected with individual (s1-s4), pooled OR10H1 siRNA or control siRNA (PD-L1 as positive and non-specific as negative control) and TIL-mediated lysis was measured by Luc-CTL cytotoxicity assay (cumulative data). C Chromium-release assay showing specific lysis of M579-A2 by TIL209 at different E:T ratios after transfection with OR10H1 siRNA 1 (Δ), positive control PD-L1 siRNA (∘) or control siRNA (▪). D Luc-CTL assay with an autologous pair of melanoma (M615-luc) and TIL (TIL615) conducted in parallel to C (cumulative data). E, F Chromium-release assay showing, respectively, lysis of SW480 colorectal cancer and PANG-1 pancreatic adenocarcinoma cells by patient-derived HLA-matched TILs at different E:T ratios upon OR10H1 (Δ), PD-L1 positive control (∘) or control (●) knockdown. All experiments were performed in triplicates and are representative (if not indicated otherwise) of at least three independent experiments. Error bars denote ±SEM, and statistical significance was calculated using unpaired, two-tailed Student's t-test.

FIG. 2: OR10H1 inhibits TIL type I cytokine secretion and induces apoptosis. A Cumulative data of type I vs. type II cytokine secretion of TIL412 after 20 h co-culture with OR10H1 knock-down (siRNA1 or “s1”) or OR10H1 expressing (control siRNA) M579-A2 measured by Luminex assay. B ELISpot assay showing the number of IFN-gamma secreting TILs (spot numbers) after co-culture with OR10H1 knockdown M579-A2 (or control siRNA). TILs without stimulation were used as a negative control. C cumulative data of apoptosis induction (measured by FACS staining for Annexin V⁺) in CD8⁺ TILs after co-culture (6h) with OR10H1 knock-down (siRNA1 or siRNA pool) or OR10H1 expressing (control siRNA) M579-A2. TILs stimulated with PMA/ionomycin and unstimulated TILs (no melanoma encounter) were used as positive and negative controls, respectively. A and C show cumulative data from three independent experiments. B shows representative data of two independent experiments (performed in triplicates). Error bars denote ±SEM, and statistical significance was calculated using unpaired, two-tailed Student's t-test.

FIG. 3: OR10H1 functions as an immune checkpoint in vivo. A Stable OR10H1 knock-down M579-A2 (transduced with OR10H1-targeting shRNA) or control transduced M579-A2 (non-targeting sequence shRNA; NTS) were injected subcutaneously (mixed with matrigel) into the left and right flank of NSG mice. On day 2 and 9, mice (n=6) received adoptive cell transfer of TIL412 (i.v. injection) and the tumor growth was measured. Mice (n=4) with tumors but without ACT serve as a control group for tumor growth. B, C Tumor growth curves showing mean±SEM tumor volume of OR10H1-negative (i.e. OR10H1 knockdown; kd) or OR10H1-positive (i.e. NTS shRNA control) M579-A2 tumors in TIL412-treated mice, i.e., with ACT (B), or in the growth control group without ACT (C). Statistical difference was calculated using the Mann-Whitney U test.

FIG. 4: OR10H1 reduces Lck and increases CREB activity by cAMP signaling via PKA in TILs. A, B TIL412 were co-cultured with OR10H1-positive (control siRNA) or OR10H1-negative (OR10H1 siRNA) M579-A2 cells for 10 h and mRNAs were extracted from the respective TILs for transcriptome analysis by RNA sequencing. A Smear plot showing differential gene expression (log 2 of fold change) between OR10H-negative (kd)- and OR10H1-positive, control siRNA)-tumor cell-treated TILs versus the average expression (log of count per million). Log FC cutoff at ±0.5 is represented by horizontal lines. Genes with a false discovery rate (FDR) above 0.05 (depicted in gray) were excluded from subsequent analysis. B Functional enrichment analysis (Ingenuity pathway analysis; IPA) based on top upregulated (Log FC>0.5) and downregulated (Log FC<−0.5) genes with FDR 0.05. Differential gene expression-associated functions were selected based on enrichment p-value (−log of p-value; threshold=−1.3) and activation Z-score (threshold ±2). RNA-Sequencing was performed in duplicates. C, D, E, F Phospho pathway analysis for key signaling proteins in TILs after co-culture (up to 2 h) with OR10H1-positive (i.e. control siRNA) and OR10H1-negative (i.e. OR10H1 siRNA) M579-A2. Phosphoplex analysis showing phosphorylation state changes in TILs upon encountering OR10H1-positive- or OR10H1-negative M579-A2 tumor cells (log 2 ratio) of classical (C) or T cell receptor (TCR)-associated (D, E) signaling proteins (CREB and Lck) compared to unstimulated TILs. In each case, TILs stimulated with PMA and ionomycin serve as positive control. F Immunoblot analysis showing phospho-CREB (Ser133), phospho-ATF1, phospho-PKA (Thr197) and phospho-Lck (Tyr505) levels in OR10H1-positive tumor cell-treated, OR10H1-negative tumor cell-treated, PMA and ionomycin-treated or unstimulated TILs using the according phospho-specific antibodies. Beta-actin was used as a loading control. C, D and E are cumulative data of three independent experiments. F is representative of three independent experiments. Mean±SEM are shown, unless stated otherwise, and statistical significance was calculated using unpaired, two-tailed Student's t-test. G Lck inhibition by a small molecule abrogates OR10H1 knockdown effect on T-cell mediated cytotoxicity (as reflected by the ratio of cytotoxicity/viability) of melanoma cells. H Western blot showing that olfactory receptor signaling activates a unique G protein (G-alpha-Olf) and subsequently adenylate cyclase type III. I In the presence of TILs the cAMP response in M579-A2 cells is reduced if OR10H1 is knocked-down on the melanoma cells. J Pretreatment with cholera but not pertussis toxin abrogates killing of OR10H1-deficient melanoma. Representative luciferase killing assays (of at least three independent experiments) after pretreatment with pertussis (i) or cholera toxin +IBMX (ii). M579-A2-luc cells were reverse transfected as before. Before co-culture with TIL209 cells were pretreated with different concentrations of pertussis (6 h) and cholera toxin (1 hour). Treated OR10H1-positive or OR10H1-negative M579-A2-luc were co-cultured with TIL209 for 20 h. TIL-mediated tumor lysis is represented by the luciferase intensity ratio between cytotoxicity and viability (no TILs). K Principle of cAMP assay based on G-protein activation of adenylate cyclase and accumulation of cAMP by PDE inhibition. M Proposed mode of action for OR10H1-mediated inhibition of CD8+ TIL functionality.

FIG. 5: Generation of monoclonal blocking antibodies against OR10H1. Mother clones of OR10H1-blocking antibodies were generated by genetic immunization of three rats with an OR10H1-IgG construct and screening for binding and blocking activity. The four most promising mother clones were enriched and tested for their impact on TIL-mediated killing by Luc-CTL cytotoxicity assay (A and B) and for two of such clones in an IncuCyte cytotoxicity assay (C and D), based on caspase activation. A, B Blocking efficacy was tested using different antibody concentration (0-500 ug/ml) for the co-culture of TIL412 with OR10H1-positive- or OR10H1-negative M579-A2-luc, compared to pre-immunization rat serum. IncuCyte cytotoxicity assays depicting the impact of mother clones 8A11 (C) and 1611 (D) on TIL-mediated induction of apoptosis in M579-A2 over 48 hours (measurement every 5 min). The Y-axis represents the number of green object counts (event of caspase-3 activation) per well. E, F, G Summary of screening for monoclonal antibodies derived from aforementioned mother clones. Luc-CTL cytotoxicity assays showing the impact of monoclonal supernatants (unknown concentration) on the TIL-mediated killing of wild-type (E), control knockdown (F) or OR10H1 knockdown (G) M579-A2-luc. A and B are representative of two independent experiments. C and D are exemplary experiments. E, F and G are results from one screening. A-E were performed in triplicates, F and G were performed in duplicates. Error bars denote +/−SEM, and statistical significance was calculated using unpaired, two-tailed Student's t-test with * p≤0.05; ** p≤0.01; *** p≤0.001; **** p≤0.0001. Clone IDs: A=1C3, B=1B11, C=8A11, D=4B4, A-1=1C3-B2, A-2=1C3-A9, D-1=4B4-A10, C-1=8A11-G12, C-2=8A11-E7, B-1=1B11-B12 and B-2=1B11-B9.

FIG. 6: Detection of OR10H1 using an antibody of the invention. A Surface expression of OR10H1 on melanoma M579-A2 transfected with non-specific control siRNA or OR10H1 siRNA 1 detected with post-immunisation polysera against OR10H1. B Surface expression of OR10H1 detected by the antibody purified from clone Di-8A11-H12-E6 on M579-A2 melanoma cells that express OR10H1 (determined by RT-PCR), but not detected on KMM-1 myeloma cells that do not express OR10H1 (determined by RT-PCR).

FIG. 7: Amino acid sequences of certain antibodies of or for use in the present invention. A. Sequence of the amino acid sequence of the heavy and light chains of antibody 1C3-A1-A1; B. Sequence of the amino acid sequence of the heavy and light chains of antibody 1C3-A1-A2; C. Sequence of the amino acid sequence of the heavy and light chains of antibody 8A11-B9-A1.

FIG. 8: OR10H1 mediates cAMP response in a reporter cell line suitable for small-molecule screening. A A non-tumour HEK293 reporter cell line express OR10H1 on their surface (detected by FLAG-tag FACS data) after transfection with a suitable OR10H1-encoding genetic construct. B Assay principle of Example 11 (adapted from Hill et al 2001, Curr Opin Pharmacol 1:526). C cAMP production was significantly increased (as measured by Cre-luc activity, and upon treatment with 10 uM forskolin (not for the “basal” group)) in such OR10H1-expressing HEK293 reporter cells compared to control-transfected HEK393 reporter cells known to surface-express the G-protein coupled receptor GPR41.

The present invention, and particular non-limiting aspects and/or embodiments thereof, can be described in more detail as follows:

### DEFINITIONS

In order that the present invention may be readily understood, several definitions of terms used in the course of the invention are set forth below.

The term “antigen” refers to a molecule or a portion of a molecule capable of being bound by an agent that specifically binds to such molecule or portion thereof, such as an antigen binding protein (including, e.g., an antibody or binding fragment thereof). An antigen can possess one or more epitopes that are capable of interacting with different antigen binding proteins, e.g., antibodies.

The term “epitope” includes any determinant capable of being bound by an antigen binding protein, such as an antibody. An epitope is a region of an antigen that is bound by an antigen binding protein that targets that antigen, and when the antigen is a protein, includes specific amino acids that bind the antigen binding protein (such as via an antigen binding domain of said protein). Epitope determinants can include chemically active surface groupings of molecules such as amino acids, sugar side chains, phosphoryl or sulfonyl groups, and can have specific three dimensional structural characteristics, and/or specific charge characteristics. Generally, antigen binding proteins specific for a particular target antigen will preferentially recognize an epitope on the target antigen in a complex mixture of proteins and/or macromolecules.

An “antigen binding protein” (“ABP”) as used herein means a protein that specifically binds one or more epitope(s) displayed by or present on a target antigen. The antigen of the ABPs of the invention is OR10H1 or a paralogue, orthologue or other variant thereof; and the epitope(s) is/are displayed by or present on one or more extracellular domains of said OR10H1 or variant. Typically, an antigen binding protein is an antibody (or a fragment thereof), however other forms of antigen binding protein are also envisioned by the invention. For example, the ABP may be another (non-antibody) receptor protein derived from small and robust non-immunoglobulin “scaffolds”, such as those equipped with binding functions for example by using methods of combinatorial protein design (Gebauer & Skerra, 2009; Curr Opin Chem Biol, 13:245). Particular examples of such non-antibody ABPs include: Affibody molecules based on the Z domain of Protein A (Nygren, 2008; FEBS J 275:2668); Affilins based on gamma-B crystalline and/or ubiquitin (Ebersbach et al, 2007; J Mo Biol, 372:172); Affimers based on cystatin (Johnson et al, 2012; Anal Chem 84:6553); Affitins based on Sac7d from Sulfolobus acidcaldarius (Krehenbrink et al, 2008; J Mol Biol 383:1058); Alphabodies based on a triple helix coiled coil (Desmet et al, 2014; Nature Comms 5:5237); Anticalins based on lipocalins (Skerra, 2008; FEBS J 275:2677); Avimers based on A domains of various membrane receptors (Silverman et al, 2005; Nat Biotechnol 23:1556); DARPins based on an ankyrin repeat motif (Strumpp et al, 2008; Drug Discov Today, 13:695); Fynomers based on an SH3 domain of Fyn (Grabulovski et al, 2007; J Biol Chem 282:3196); Kunitz domain peptides based on Kunitz domains of various protease inhibitors (Nixon et al, Curr opin Drug Discov Devel, 9:261) and Monobodies based on a 10th type III domain of fibronectin (Koide & Koide, 2007; Methods Mol Biol 352:95).

The term “extracellular” domain (“EC” domain) as used herein refers to the region or regions of the protein which are exposed to the extracellular space and which are typically responsible for ligand binding. GPCRs (the class of proteins to which OR10H1 belongs) typically have four extracellular domains, which connect to seven transmembrane (TM) domains which are a common structural signature of GPCRs, as too are the four EC domains, the first being an extracellular amino terminus, and the four intracellular domains, the last being an intracellular carboxyl terminus. GPCRs share the greatest homology within the TM segments. The most variable structures among the family of GPCRs are the carboxyl terminus, the intracellular loop spanning TM5 and TM6, and the amino terminus. The greatest diversity is observed in the first EC domain being the amino terminus of the receptor (Kobilka 2007; Biochim Biophys Acta. 1768:794).

An “antigen binding domain” as used herein means the part of an ABP (sequence of amino acids) that interacts most closely with (e.g. binds) the target antigen, such as at the epitope.

The term “compete” when used in the context of ABPs (e.g., modulator ABPs) that compete for the same epitope means competition between ABPs as determined by an assay in which the ABP (e.g., antibody or binding fragment thereof) being tested prevents or inhibits (e.g., reduces) binding of a reference ABP (e.g., a ligand, or a reference antibody) to a common antigen (e.g., OR10H1 or a fragment thereof).

The term “amino acid” includes its normal meaning in the art.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm.

A standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) may also be used by the algorithm.

Examples of parameters that can be employed in determining percent identity for polypeptides or nucleotide sequences using the GAP program are the following:
Algorithm: Needleman et al., 1970, J. Mol. Biol. 48:443-453
Comparison matrix: BLOSUM 62 from Henikoff et al., 1992, supra
Gap Penalty: 12 (but with no penalty for end gaps)
Gap Length Penalty: 4
Threshold of Similarity: 0

A preferred method of determining similarity between a protein or nucleic acid and (or between) human OR10H1, a paralogue, orthologue or other variant thereof, is that provided by the Blast searches supported at HORDE supra (e.g., https://genome.weizmann.ac.il/cgi-bin/horde/blastHorde.pl); in particular for amino acid identity, those using BLASTP 2.2.28+ with the following parameters: Matrix: BLOSUM62; Gap Penalties: Existence: 11, Extension: 1; Neighboring words threshold: 11; Window for multiple hits: 40.

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least about 10, 15, 20, 25, 30, 35, 40, 45, 50 or other number of contiguous amino acids of the target polypeptide or region thereof.

The term “paralogue” as used herein means a variant in the same organism that descends from the same ancestral gene by a duplication event. A paralogue of OR10H1 is typically expected to be an olfactory receptor. Those paralogues of human OR10H1 having the closest similarity thereto are: OR10H5 (313 amino acids; 93.6% identity, SEQ ID NO: 14) and OR10H2 (307 amino acids; 89.6% identity, SEQ ID NO: 15). OR10H2 (aliases: OR19-23, AC004597-A, HsOR19.4.1, ORL312, ORL528; Database IDs: HGNC: 8173; GeneBank: AF399585, BC069085, BC069457, BK004211, NM_013939; ORDB: ORL312, ORL528; Chromosome location: Chr19:15,728,044-15,728,988(+), GRCh38/hg38; UniProtKB identifier: 060403, Sequence version 1 of 1 Aug. 1998, Entry version 130 of 5 Oct. 2016); OR10H5 (aliases: OR19-25, OR19-26, HsOR19.4.3, ORL3761; Database IDs: HGNC: 15389; GeneBank: AB065920, AF399583, BK004271, BK004272, NM_001004466; ORDB: ORL3761; Chromosome location: Chr19:15,794,049-15,794,993(+), GRCh38/hg38; UniProtKB identifier: Q8NGA6, Sequence version 1 of 1 Oct. 2002, Entry version 116).

The term “orthologue” as used herein means a variant that descends from the same ancestral gene but which is present in another organism due to a speciation event. An orthologue of OR10H1 is typically expected to retain the same function as (or have a similar function to) human OR10H1. Those orthologues of human OR10H1 having the closest similarity thereto include those of chimpanzee (307 amino acids; 89% identity), cow (314 amino acids; 89% identity), mouse (310 amino acids; 87% identity) and rat (300 amino acids; 87% identity). A particular orthologue of human OR10H1 is that of cynomolgus monkeys. An example of a cynomolgus monkey orthologue of human OR10H1 is that according to SEQ ID NO: 11 (317 amino acids; 94% identity) (UniProt ID: G7PZP4, Sequence version 1 of 25 Jan. 2012, Entry version 18 of 11 May 2016).

The term “variant” as used herein in the context of OR10H1 means any natural or non-natural OR10H1 which comprises one or more amino acid mutations compared to human OR10H1 but which shares significant amino acid sequence identity with human OR10H1, e.g. at least 70% amino acid sequence identity, preferably at least 80% amino acid sequence identity, more preferably at least 90% amino acid sequence identity and most preferably at least 95%, 96%, 97%, 98% or 99% amino acid sequence identity. Variants of OR10H1 may include paralogues, orthologues and natural variants of human OR10H1, such as G16R, A65V (including G16R together with A65V), A142T, A167T, H175Q and others. Variants of OR10H1 may also correspond to human OR10H1 with one or more amino acid residues inserted into, or deleted from the amino acid sequence, such as those variants of OR10H1 naturally found within a population or those made by genetic manipulation, such as to specifically engineer amino acid changes into one or more domains (such as extracellular domains) of the variant. Variants of OR10H1 include fusion proteins of OR10H1 (for example, a human OR1H1 fused to a heterologous polypeptide chain), and/or OR10H1 conjugated to another chemical moiety such as an effector group or a labelling group. A variant of OR10H1 can, in certain embodiments, comprise a fragment of OR10H1, for example a polypeptide that consists of one or more EC domains (or regions thereof) of OR10H1 without one or other (or any other) EC, TM or intracellular domains of OR10H1. Preferred such variants of OR10H1 that are fragments include those that comprise the 1^{st}, 2^{nd}, 3^{rd} and/or 4^{th} EC domain of OR10H1 without any of the TM or intracellular domains of OR10H1; more preferably those that comprise the 2^{nd} EC domain of OR10H1 without one or more (or all) of the other EC, TM or intracellular domains of OR10H1. Variants of OR10H1 also include versions of human OR10H1 (or paralogues or orthologues thereof) that have been modified to display only specific domains (such as extracellular), or not to display one or more other domains, and/or to display certain (e.g. EC) domains of human OR10H1 in combination with domains from paralogues and/or orthologues of human OR10H1, or from other GPCRs. Methods describing the engineering of domain or amino acid variants of OR10H1 are described in the examples herein. In certain embodiments, the variant of OR10H1 is a functional variant thereof. A “functional variant” of OR10H1 (such as a functional fragment of an OR10H1 protein) is a variant of the protein of OR10H1 that provides, possesses and/or maintains one or more of the herein described functions/activities of the non-variant protein of OR10H1. For example, such functional variant may bind one or more of the same chemostimuli as OR10H1 protein, may signal the same G protein-coupled adenylyl cyclase cascade as the OR10H1 protein and/or may be coupled to one or more of the same G-alpha-s and G-alpha-15 G proteins as OR10H1 protein, such as having the same, essentially the same or similar specificity and/or function as a receptor as OR10H1 protein. In other embodiments, such a functional variant may possess other activities than those possessed by the non-variant OR10H1 protein, as long as, preferably, it provides, possesses and/or maintains at least one function/activity that is the same, essentially the same or similar as OR10H1 protein. In more preferred embodiments, a functional variant of OR10H1 may act as an immune checkpoint inhibitor, such as by inhibiting one or more cell-based immune response(s) to a tumour or cancer cell that expresses such functional variant.

The term “isolated” as used herein in the context of a protein, such as an ABP (an example of which could be an antibody), refers to a protein that is purified from proteins or polypeptides or other contaminants that would interfere with its therapeutic, diagnostic, prophylactic, research or other use. An isolated ABP according to the invention may be a recombinant, synthetic or modified (non-natural) ABP. The term “isolated” as used herein in the context of a nucleic acid or cells refers to a nucleic acid or cells that is/are purified from DNA, RNA, proteins or polypeptides or other contaminants (such as other cells) that would interfere with its therapeutic, diagnostic, prophylactic, research or other use, or it refers to a recombinant, synthetic or modified (non-natural) nucleic acid. Preferably an isolated ABP or nucleic acid or cells is/are substantially pure. In this context, a “recombinant” protein or nucleic acid is one made using recombinant techniques. Methods and techniques for the production of recombinant nucleic acids and proteins are well known in the art.

As used herein, “substantially pure” or “purified” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, 96%, 97%, 98%, 99%, 99.5% or 99.8% of all macromolecular species present in the composition. In certain embodiments, an essentially homogeneous substance has been purified to such a degree that contaminating species cannot be detected in the composition by conventional detection methods and thus the composition consists of a single detectable macromolecular species.

An antigen binding protein is “specific” when it binds to one antigen more preferably than it binds to a second antigen. The term “specifically binds” (or “binds specifically”) used herein in the context of an ABP of the invention means that said ABP will preferably bind to said OR10H1 or variant than to other proteins (or other molecules), such as preferably binding to said OR10H1 or variant compared to one or more of its paralogues, orthologues and/or other GPCRs (such as, in each case, extracellular domains thereof). Therefore, preferably, the binding affinity of the ABP to the one antigen (e.g. human OR10H1) is at least 2-fold, 5-fold, at least 10-fold, at least 20-fold, at least 50-fold, at least 100-fold, at least 200-fold, at least 500-fold, at least 1000-fold, at least 2000-fold, at least 5000-fold, at least 10000-fold, at least 10⁵-fold or even at least 10⁶-fold, most preferably at least 2-fold, compared to its affinity to the other targets (e.g. paralogues of human OR10H1, such as OR10H2 and/or OR10H5).

As used herein, the term “antibody” may be understood in the broadest sense as any immunoglobulin (Ig) that enables binding to its epitope. An antibody as such is a species of an ABP. Full length “antibodies” or “immunoglobulins” are generally heterotetrameric glycoproteins of about 150 kDa, composed of two identical light and two identical heavy chains. Each light chain is linked to a heavy chain by one covalent disulphide bond, while the number of disulphide linkages varies between the heavy chain of different immunoglobulin isotypes. Each heavy and light chain also has regularly spaced intrachain disulphide bridges. Each heavy chain has an amino terminal variable domain (VH) followed by three carboxy terminal constant domains (CH). Each light chain has a variable N-terminal domain (VL) and a single C-terminal constant domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to cells or factors, including various cells of the immune system (e.g., effector cells) and the first component (C1q) of the classical complement system. Other forms of antibodies include heavy-chain antibodies, being those which consist only of two heavy chains and lack the two light chains usually found in antibodies. Heavy-chain antibodies include the hcIgG (IgG-like) antibodies of camelids such as dromedaries, camels, llamas and alpacas, and the IgNAR antibodies of cartilaginous fishes (for example sharks). And yet other forms of antibodies include single-domain antibodies (sdAb, called Nanobody by Ablynx, the developer) being an antibody fragment consisting of a single monomeric variable antibody domain. Single-domain antibodies are typically produced from heavy-chain antibodies, but may also be derived from conventional antibodies.

The term “monoclonal antibody” or “mAb” as used herein refers to an antibody obtained from a population of substantially homogeneous antibodies based on their amino acid sequence. Monoclonal antibodies are typically highly specific. Furthermore, in contrast to conventional (polyclonal) antibody preparations which typically include different antibodies directed against different determinants (e.g. epitopes) of an antigen, each mAb is typically directed against a single determinant on the antigen. In addition to their specificity, mAbs are advantageous in that they can be synthesized by cell culture (hybridomas, recombinant cells or the like) uncontaminated by other immunoglobulins. The mAbs herein include for example chimeric, humanized or human antibodies or antibody fragments.

The term “chimeric antibody” according to the present invention refers to an antibody whose light and/or heavy chain genes have been constructed, typically by genetic engineering, from immunoglobulin variable and constant regions which are identical to, or homologous to, corresponding sequences of different species, such as mouse and human. Alternatively, heavy chain genes derive from a particular antibody class or subclass while the remainder of the chain derives from another antibody class or subclass of the same or a different species. It covers also fragments of such antibodies. For example, a typical therapeutic chimeric antibody is a hybrid protein composed of the variable or antigen-binding domain from a mouse antibody and the constant or effector domain from a human antibody, although other mammalian species may be used.

The term “humanized antibody” according to the present invention refers to specific chimeric antibodies, immunoglobulin chains or fragments thereof (such as Fab, Fab′, F(ab′)₂, Fv, or other antigen-binding sub-sequences of antibodies), which contain minimal sequence (but typically, still at least a portion) derived from non-human immunoglobulin. For the most part, humanized antibodies are human immunoglobulins (the recipient antibody) in which CDR residues of the recipient antibody are replaced by CDR residues from a non-human species immunoglobulin (the donor antibody) such as a mouse, rat or rabbit having the desired specificity, affinity and capacity. As such, at least a portion of the framework sequence of said antibody or fragment thereof may be a human consensus framework sequence. In some instances, Fv framework residues of the human immunoglobulin need to be replaced by the corresponding non-human residues to increase specificity or affinity. Furthermore, humanized antibodies can comprise residues which are found neither in the recipient antibody nor in the imported CDR or framework sequences. These modifications are made to further refine and maximize antibody performance. In general, the humanized antibody will comprise substantially all of at least one, and typically at least two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region, typically that of a human immunoglobulin.

As used herein, a “mammalian cell”, such as one expressing OR10H1, or a paralogue, orthologue or other variant thereof, may be from dogs, cats, horses, sheep, goats, cows, pigs, rabbits, rodents (e.g., mice or rats) and primates, such as cynomolgus monkeys, chimpanzees, great apes and humans. The mammalian cell may also be an engineered cell. Preferably the mammalian cell is a human cell.

ABPs of the invention can be “modulators”. The term “modulator” as used herein, refers to a molecule that changes, modifies or alters one or more characteristics, properties and/or abilities of another molecule or, for example, that changes, modifies or alters an immune response (“immunomodulators”), such as a cell-mediated immune response. For example, a modulator can impair or interfere with, or cause a decrease in the magnitude of, expression, function, activity and/or stability, such as a certain activity or function, of a molecule compared to the magnitude of such characteristic, property or ability observed in the absence of the modulator. Certain exemplary characteristics, properties or abilities of a molecule include, but are not limited to, expression, function, activity and/or stability, such as binding affinity, enzymatic activity, and signal transduction; for example any of the functions or activities of OR10H1 described herein.

Immunomodulatory ABPs can act as “inhibitors” (“antagonists”) against a receptor, such as by impairing (e.g. blocking) ligand engagement.

As used herein, the terms “modulator of OR10H1 expression” and the like (such as an “inhibitor [or antagonist] of OR10H1 expression” and the like) shall relate to any of the herein disclosed ABPs which has an effect (such as an antagonistic activity) toward the expression of an OR10H1 protein, that is it alters (e.g. impairs, suppresses, reduces and/or lowers) the expression of an OR10H1 protein such as may be determined by measuring an amount (or change in an amount) of OR10H1 protein or OR10H1 mRNA. The term “expression” means in this context the cellular process of transcribing a gene into an mRNA and the following translation of the mRNA into a protein. “Gene expression” therefore may thus refer only to the generation of mRNA, irrespectively from the fate of the so produced mRNA, or alternatively/additionally to the translation of the expressed mRNA into a protein. The term “protein expression” on the other hand may refer to the complete cellular process of synthesis of proteins. The terms “modulator of expression of a [orthologue][paralogue][variant] of OR10H1” and the like, shall have the corresponding meaning with respect to any such variant of OR10H1.

The terms a “modulator of OR10H1 function [or activity]” and the like (such as an “inhibitor [or antagonist] of OR10H1 function [or activity]” and the like) shall refer to any of the herein disclosed ABPs that alters, such as impairs (e.g., induces a decrease or reduction in) the efficiency, effectiveness, amount or rate of one or more activities of OR10H1 (for example, by impairing the expression and/or stability of OR10H1 protein), such as one or more of those activities described herein, for example, the activity of OR10H1 as a modulator of T-cell activation and/or viability. In one embodiment, such a modulating ABP may impair binding of one or more of the chemostimuli of OR10H1 protein, may impair signaling by the G protein-coupled adenylyl cyclase cascade of OR10H1 protein and/or may impair the coupling or activity of one or more of the G-alpha-s and G-alpha-15 G proteins of OR10H1 protein. The terms “modulator of function of a [orthologue][paralogue][variant] of OR10H1” and the like, shall have the corresponding meaning with respect to any such variant of OR10H1.

The terms “modulator of OR10H1 stability” and the like (such as an “inhibitor [or antagonist] of OR10H1 stability” and the like) shall refer to any of the herein disclosed ABPs which has an effect (such as a negative activity) towards the stability of an OR10H1 protein. The term, in context of the present disclosure, shall be understood in its broadest sense. Such modulators are included by the term, which, for example, interfere with and reduce the OR10H1 protein half-live or interfere with and disturb OR10H1 protein folding or protein presentation on the surface of the cell. In one preferred example, an inhibiting modulator of the invention, such as an ABP, may induce internalisation, and optionally degradation, of OR10H1 protein from the surface of the cell. The terms “modulator of stability of a [orthologue][paralogue][variant] of OR10H1” and the like, shall have the corresponding meaning with respect to any such variant of OR10H1.

The term “cytotoxic T cell” and its abbreviation “CTL” as used herein may be understood in the broadest sense as any T lymphocyte that is able to induce cell death, in particular in neoplastic cells, cells that are infected, particularly viruses-infected cells, and/or cells in other pathological conditions. In this context, the terms “cytotoxic T cell”, “CTL”, “cytotoxic TC”, “cytotoxic T lymphocyte”, “T killer cell”, “cytolytic T cell” and “killer T cell” may be understood interchangeably. The cytotoxic T cell may be a cytotoxic CD8+ T cell. Typically, a CTL in the context of the present invention has at least one T cell receptor (TCR) on its surface that enables the recognition of particular molecular structures presented at surfaces of other cells. Those molecular structures will typically be antigens presented at the surface of the other cell in complex with major histocompatibility complex (MHC) class I, where they can be recognized by the CTL. If the TCR is specific for that antigen, it will bind to said complex of the MHC class I with the antigen and a CTL response occurs, i.e., the other cell is destroyed. Preferably, the CTLs used in the context of the present invention are mammalian CTLs, in particular human CTLs, so that the CTL response is a human CTL response. Tumour infiltrating lymphocytes (TILs) are lymphocytes that have left the bloodstream and migrated into a tumour. They may be a mix of different types of cells (i.e., T cells, B cells, NK cells, macrophages) in variable proportions, T cells being the most abundant cell. TILs are implicated in killing tumour cells. The presence of lymphocytes in tumours is often associated with better clinical outcomes. According to one aspect of the invention, modulation of OR10H1 expression, function, activity and/or stability in tumour cells will modulate the TIL response to such tumour cells.

The terms “polynucleotide”, “oligonucleotide” and “nucleic acid” are used interchangeably throughout and include DNA molecules (e.g., cDNA or genomic DNA), RNA molecules (e.g., mRNA), analogues of the DNA or RNA generated using nucleotide analogues (e.g., peptide nucleic acids and non-naturally occurring nucleotide analogues), and hybrids thereof. The nucleic acid molecule can be single-stranded or double-stranded.

A “nucleic acid construct” (“NAC”) is a nucleic acid, such as a “vector” that can be used to propagate, produce, maintain or introduce a nucleic acid comprised within it in/into a cell, such as for expression of a polypeptide encoded by a sequence comprising said nucleic acid. One type of NAC is a “plasmid”, which refers to a linear or circular double stranded DNA molecule into which additional nucleic acid segments can be ligated. Another type of NAC is a viral vector (e.g., replication defective retroviruses, adenoviruses and adeno-associated viruses), wherein additional DNA segments can be introduced into the viral genome. Certain NACs are capable of autonomous replication in a cell, such as a host cell, into which they are introduced (e.g., bacterial vectors comprising a bacterial origin of replication and episomal mammalian vectors). Other NACs (e.g., non-episomal mammalian vectors) integrate into the genome of a cell upon introduction into the cell and culturing under selective pressure, and thereby are replicated along with the genome. A NAC can be used to direct the expression of a chosen polynucleotide in a cell. A NAC may comprise an mRNA molecule which includes an open reading frame encoding a polypeptide of interest, together with one or more upstream and/or downstream elements (such as 5′ and/or 3′ UTRs and/or a poly-A stretch) that enable expression of the polypeptide, and preferably enhance stability of the mRNA and/or expression of the desired polypeptide. Exemplary mRNA NACs are described by Zangi et al in Nat. Biotechnol. vol. 31, 898-907 (2013), Sahin et al (2014) Nature Reviews Drug Discovery 13:759 and by Thess et al in Mol. Ther. vol. 23 no. 9, 1456-1464 (2015). In particular, the use of mRNA therapeutics for the expression of antibodies is known from WO2008/083949. NACs such as DNA-, retroviral- and mRNA-based NACs may be used in genetic therapeutic methods, where instead of administering to the cell or organism the desired polypeptide (such as an ABP of the present invention), a NAC that comprises an expressible sequence encoding such desired polypeptide is administered to the cell or organism (e.g. by transfection). The NAC is incorporated into a cell and the cell's own expression machinery transcribes and translates (for DNA-based NACs) or only translates (for RNA-based NACs) the encoded polypeptide.

A “host cell” is a cell that can be used to carry, propagate and/or express a nucleic acid, e.g., a nucleic acid or DNA of the invention and the respective protein. A host cell can be a prokaryote, for example, E. coli, or it can be a eukaryote, for example, a single-cell eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Exemplary host cells include Chinese hamster ovary (CHO) cell lines or their derivatives. Typically, a host cell is a cultured cell that can be transformed or transfected with a polypeptide-encoding nucleic acid, which can then be expressed in the host cell. However, a host cell may also be a cell isolated from a specific subject (such as a human subject or patient), and then such cell is propagated and/or modified so that it may carry, propagate and/or express a nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “composition” means a mixture of substances. The term “pharmaceutical composition” means a mixture of substances including the therapeutically active substance, such as ABPs or NACs of the invention, for pharmaceutical use.

The term “label” or “labelling group” refers to any detectable label. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.).

The term “effector group” means any group, in particular one coupled to another molecule such as an antigen binding protein, that acts as a cytotoxic agent. Examples for suitable effector groups are radioisotopes or radionuclides. Other suitable effector groups include toxins, therapeutic groups, or chemotherapeutic groups. Examples of suitable effector groups include calicheamicin, auristatins, geldanamycin and maytansine.

In some embodiments, an effector group or a labelling group is coupled to another molecule (such as the ABP) via spacer arms of various lengths to reduce potential steric hindrance.

As used herein, “a subject” includes all mammals, including without limitation humans, but also non-human primates such as cynomolgus monkeys. It also includes dogs, cats, horses, sheep, goats, cows, rabbits, pigs and rodents (such as mice and rats). It will be appreciated that a particularly preferred subject according to the invention is a human subject, such as a human suffering from (or at risk of suffering from) a disorder, disease or condition, for example a human patient.

As used herein, “therapy” is synonymous with treating a disease, disorder or condition, which includes reducing symptoms of the disease, disorder or condition, inhibiting progression of the disease, disorder or condition, causing regression of the disease, disorder or condition and/or curing the disease, disorder or condition.

The term “treatment” in the present invention is meant to include therapy, e.g. therapeutic treatment, as well as prophylactic or suppressive measures for a disease (or disorder or condition). Thus, for example, successful administration of an ABP prior to onset of the disease results in treatment of the disease. “Treatment” also encompasses administration of an ABP after the appearance of the disease in order to ameliorate or eradicate the disease (or symptoms thereof). Administration of an ABP after onset and after clinical symptoms, with possible abatement of clinical symptoms and perhaps amelioration of the disease, also comprises treatment of the disease. Those “in need of treatment” include mammals (such as a human subject) already having the disease, disorder or condition, as well as those prone to or suspected of having the disease, disorder or condition, including those in which the disease, disorder or condition is to be prevented.

The term “immune cell” is art recognised to describe any cell of an organism involved in the immune system of such organism, in particular of a mammal such as a human. Leukocytes (white blood cells) are immune cells that are involved in the innate immune system, and the cells of the adaptive immune system are special types of leukocytes, known as lymphocytes. B cells and T cells are the major types of lymphocytes and are derived from hematopoietic stem cells in the bone marrow. B cells are involved in the humoral immune response, whereas T cells are involved in cell-mediated immune response. In preferred embodiments of the invention, the immune cell can be T cells, and in particular (such as when an increase in cell-mediated immune response is required, such as to treat a cancer) the T cell can be a cytotoxic T cell (also known as TC, cytotoxic T lymphocyte, CTL, T-killer cell, cytolytic T cell, CD8+ T-cell or killer T cell). A CTL is a T-cell that is involved in the killing of cancer cells, cells that are infected (particularly with viruses), or cells that are damaged in other ways. Other preferred immune cells for such embodiments can include Tumour-Infiltrating Lymphocytes (TILs). TILs are white blood cells that have left the bloodstream and migrated into a tumour. Typically, TILs are a mix of different types of cells (i.e., T cells, B cells, NK cells, macrophages) in variable proportions, T cells being the most abundant cells. TILs can often be found in the stroma and within the tumour itself, and are implicated in killing tumour cells. The presence of lymphocytes in tumours is often associated with better clinical outcomes.

As used herein, the term “fluorescence activated cell sorting” or “FACS” refers to a method by which the individual cells of a sample are analyzed and sorted according to their optical properties (e.g., light absorbance, light scattering and fluorescence properties, etc.) as they pass in a narrow stream in single file through a laser beam. FACS of live cells separates a population of cells into sub-populations based on fluorescent labelling. Cells stained using fluorophore-conjugated antibodies can be separated from one another depending on which fluorophore they have been stained with. For example, a cell expressing one cell marker may be detected using an FITC-conjugated antibody that recognizes the marker, and another cell type expressing a different marker could be detected using a PE-conjugated antibody specific for that marker.

The terms “of the invention”, “in accordance with the invention”, or “according to the invention” as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

Antigen Binding Proteins

The present invention is based on the finding that OR10H1 can function as an immune modulator, in particular as an immune checkpoint receptor in tumour cells. For example, OR10H1 expressed on the surface of tumour cells inhibits the activity of certain immune cells against said tumour cells, reduces TIL type I cytokine secretion and induces apoptosis of immune cells, such as T-cells, thereby down-regulating the CTL response. Thus, modulation of OR10H1 (or of paralogues, orthologues or other variants thereof) by the antigen binding proteins of the invention may be expected to have therapeutic benefit in a number of diseases, disorders or conditions.

In one aspect, herein provided is an ABP that binds to a human OR10H1, or a paralogue, orthologue or other variant thereof, wherein said ABP binds to one or more epitope(s) displayed by one or more extracellular domain(s) of said OR10H1, paralogue, orthologue or other variant, such as when said OR10H1 or variant is expressed on the surface of a mammalian cell. In a preferred embodiment, the mammalian cell is a human cell.

An ABP of the invention can be considered to comprise at least one antigen binding domain that binds to said OR10H1 or variant, in particular an antigen binding domain that interacts with one or more specific determinants (e.g. epitope(s)) of the extracellular domain(s) of said OR10H1 or variant.

The epitope(s) to which the ABPs of the invention bind are present on or displayed by one (or more) of those particular regions of the polypeptide chain of the human OR10H1 (or a paralogue, orthologue or other variant thereof) that is typically, such as under normal physiological conditions, exposed to the extracellular space of a mammalian cell. For example, when such polypeptide is expressed by (such as on the surface of) a mammalian cell (e.g. a human cell), the region(s) of such polypeptide to which an ABP of the invention binds (such as via an interaction between an epitope displayed by said polypeptide region and an antigen binding domain comprised by the ABP) is/are presented on the outside of the membrane of the mammalian cell.

In common with other GPCRs, OR10H1 is predicted to have four extracellular domains which, according to the applicable UniProt entry accessed on 12 Oct. 2016 (http://www.uniprot.org/uniprot/Q9Y4A9), correspond to regions of the polypeptide chain of OR10H1 represented by amino acids from about 1 to about 25, from about 76 to about 99, from about 161 to about 197 and from about 260 to about 272 of human OR10H1 (SEQ ID NO: 1, 2, 3, 4 or 12). The skilled person will appreciate that these regions are estimated based on amino acid sequence properties; the boundaries between extracellular (EC) domains and transmembrane (TM) domains of OR10H1 may actually vary by up to several amino acids, such as by 1, 2, 3, 4, 5, 6, 7, 8, 9 or more than 9 amino acids positions, in particular between 1 and about 5 amino acids positions, and preferably 2 or 3 amino acids positions, as given in the preceding sentence. For example, the corresponding EC domains given by the HORDE (supra) entry accessed on 12 Oct. 2016 [https://genome.weizmann.ac.il/horde/card/index/symbol:OR10H1/] for OR10H1 are represented by amino acids from about 1 to about 23, from about 84 to about 97, from about 162 to about 197 and from about 266 to about 270 of human OR10H1. In particular, the boundaries between EC domains in a paralogue, orthologue or other variant of human OR10H1 may differ by more than about 7, 8, 9 or 10 amino acids positions, particularly for the position(s) of extracellular domains towards the C-terminal end of the polypeptide, as the cumulative effect of deletions (especially) of individual amino acids or of other regions/domains of the OR10H1, or by swapping of domains with corresponding domains from heterologous proteins, can lead to larger differences in amino acid numbering. As well as these four EC domains, the remaining regions of the chain correspond to seven TM segments and four intracellular domains (including the intracellular carboxyl terminus).

The extracellular domains of a given OR10H1 or variant will be known or readily identifiable by the person of ordinary skill. For example, information on EC domains of OR10H1 (or a given paralogue, or orthologue) may be obtained from HORDE (supra), or may be predicted from the sequence of a given variant such as by comparison of the amino acid sequence of such variant to that of one or more olfactory receptors (or other GPCR) of known structure (Man et al, 2004; Protein Sci 13:240).

In some embodiments, the ABP (e.g., comprising one antigen binding domain) binds to one or more epitope(s) displayed by one or more extracellular domain(s) of a human OR10H1, or a paralogue, orthologue or other variant thereof.

In some embodiments, the ABP binds to a single epitope displayed by one or more extracellular domain(s) of a human OR10H1, or a paralogue, orthologue or other variant thereof.

In particular embodiments, the ABP of the invention binds to (e.g. via one or more epitope(s) displayed by one or more extracellular domain(s) of) an OR10H1 variant that shares at least 80%, at least 85%, at least 90%, at least 95%, at least 96%, at least 97%, at least 98%, or at least 99% amino acid sequence identity, preferably at least 95% and more preferably at least 97% sequence identity, with the sequence of SEQ ID NO 1, or to (e.g. via one or more epitope(s) displayed by one or more one or more extracellular domain(s)) an OR10H1 that is identical to the sequence of SEQ ID NO 1. In other particular embodiments, the ABP of the invention binds to (e.g. via one or more epitope(s) displayed by one or more extracellular domain(s) of) an OR10H1 variant that shares at least 80%, at least 85%, at least 90%, at least 95%, at least 96%, at least 97%, at least 98%, or at least 99% amino acid sequence identity, preferably at least 95% and more preferably at least 97% sequence identity, with the sequence of SEQ ID NO 2, or to (e.g. via one or more epitope(s) displayed by one or more one or more extracellular domain(s)) an OR10H1 that is identical to the sequence of SEQ ID NO 2. In further other particular embodiments, the ABP of the invention binds to (e.g. via one or more epitope(s) displayed by one or more extracellular domain(s) of) an OR10H1 variant that shares at least 80%, at least 85%, at least 90%, at least 95%, at least 96%, at least 97%, at least 98%, or at least 99% amino acid sequence identity, preferably at least 95% and more preferably at least 97% sequence identity, with the sequence of SEQ ID NO 3, or to (e.g. via one or more epitope(s) displayed by one or more one or more extracellular domain(s)) an OR10H1 that is identical to the sequence of SEQ ID NO 3. In yet other particular embodiments, the ABP of the invention binds to (e.g. via one or more epitope(s) displayed by one or more extracellular domain(s) of) an OR10H1 variant that shares at least 80%, at least 85%, at least 90%, at least 95%, at least 96%, at least 97%, at least 98%, or at least 99% amino acid sequence identity, preferably at least 95% and more preferably at least 97% sequence identity, with the sequence of SEQ ID NO 4, or to (e.g. via one or more epitope(s) displayed by one or more one or more extracellular domain(s)) an OR10H1 that is identical to the sequence of SEQ ID NO 4. In yet further other particular embodiments, the ABP of the invention binds to (e.g. via one or more epitope(s) displayed by one or more extracellular domain(s) of) an OR10H1 variant that shares at least 80%, at least 85%, at least 90%, at least 95%, at least 96%, at least 97%, at least 98%, or at least 99% amino acid sequence identity, preferably at least 95% and more preferably at least 97% sequence identity, with the sequence of SEQ ID NO 12, or to (e.g. via one or more epitope(s) displayed by one or more one or more extracellular domain(s)) an OR10H1 that is identical to the sequence of SEQ ID NO 12.

Preferably in such embodiments, such sequence identity is calculated over an alignment between the respective sequences that spans the entire length of the sequences (or regions) to be compared. For example, a percentage sequence identify can be calculated (as described elsewhere herein) by consideration of the full length of the OR10H1 variant and the corresponding reference sequence of human OR10H1 (e.g. SEQ ID NOs: 1, 2, 3, 4 or 12). As an alternative, a percentage sequence identify can be calculated (also as described elsewhere herein) by consideration of an alignment that spans only the portion of each sequence that corresponds to the extracellular domain of the OR10H1 variant for which a percent identity to the equivalent extracellular domain of the reference sequence of human OR10H1 (e.g. SEQ ID NOs: 1, 2, 3, 4 or 12) can be established.

In preferred embodiments, the ABP of the invention binds to (e.g. one or more epitope(s) displayed by one or more extracellular domain(s)) of at least one human OR10H1 selected from the group consisting of SEQ ID NO: 1, SEQ ID NO: 2, SEQ ID NO: 3, SEQ ID NO: 4 and SEQ ID NO: 12.

In some embodiments, the ABP of the invention binds to one or more epitope(s) displayed by one or more extracellular domain(s) of a natural variant of OR10H1, such as those variants of OR10H1 naturally found within a population. In other embodiments, the ABP of the invention binds to one or more epitope(s) displayed by one or more extracellular domain(s) of a G16R variant of OR10H1, an A167T variant of OR10H1, and/or a H175Q variant of OR10H1.

In some embodiments, the ABP of the invention binds to one or more epitope(s) displayed by one or more extracellular domain(s) of a variant of human OR10H1 which corresponds to human OR10H1 with one or more amino acid residues inserted into, added to, or deleted from the amino acid sequence.

In some embodiments, the ABP binds to one or more epitope(s) displayed by one or more extracellular domain(s) of human OR10H1, preferably not binding to one or more extracellular domain(s) of) one or more paralogues of human OR10H1. For example, an ABP of the invention may not bind (or may not detectably bind) to one or more, or any, extracellular domain(s) of one or more (or any) paralogues of human OR10H1. In some embodiments, the ABP binds to one or more epitope(s) displayed by one or more extracellular domain(s) of human OR10H1, preferably not binding to (e.g., it does not bind to) any extracellular domain(s) of the human paralogues OR10H2 and/or OR10H5. In particular embodiments, an ABP of the invention binds the 1^{st}, 2^{nd}, 3^{rd} or4^{th} EC domain of a human OR10H1 preferably not binding to the corresponding EC domain of human OR10H2 and/or human OR10H5; and/or an ABP of the invention binds one or more EC domain(s) of a human OR10H1 preferably not binding to the 4^{th} EC domain of human OR10H2 and/or human OR10H5.

In a preferred embodiment, the ABP binds to one or more epitope(s) displayed by one or more extracellular domain(s) of human OR10H1, preferably not binding to (e.g., it does not bind to) the extracellular domain(s) of any olfactory receptor of family 1, 2 and/or 51 (OR1, OR2 and/or OR51), in particular OR1F1, OR2J2 and/or OR51E2, and/or to TAS2R3, and/or the orthologue(s) of the foregoing.

In other embodiments, the ABP of the invention, although it may indeed bind to one or more OR10H1 paralogues (such as to the 1^{st}, 2^{nd}, 3^{rd} and/or 4^{th} EC domain of said paralogue), the ABP does not modulate the expression, function, activity and/or stability of said paralogue, in particular not the expression, function, activity and/or stability of OR10H2 and/or OR10H5. In preferred embodiments, the ABP of the invention, even if it does bind to and/or modulate the expression, function, activity and/or stability of said paralogue, such binding or modulation is not associated with a modulation of an immune response, such as an enhancement or a cell-based immune response to a cancer cell that expresses such paralogue(s).

In some embodiments, the ABP of the invention binds to one or more epitope(s) displayed by one or more extracellular domain(s) of human OR10H1, preferably to binding to a plurality of other human olfactory receptors and/or over a plurality of other human GPCRs. In certain of such embodiments, the ABP of the invention does not bind (or does not detectably bind) to at least 10, 20, 30, 40 or 50 other human olfactory receptors and/or other human GPCRs.

In a preferred embodiment, the ABP of the invention does not bind (or does not detectably bind to) olfactory receptors and/or human GPCRs other than human OR10H1 and/or its naturally occurring variants.

In another preferred embodiment, the ABP of the invention binds to human OR10H1 of SEQ ID NO: 1, but does not bind to (or does not detectably bind to) human OR10H1 of SEQ ID NO: 2, SEQ ID NO: 3, SEQ ID NO: 4 or SEQ ID NO: 12; and preferably does not bind to (or does not detectably bind to) any other olfactory receptors and/or human GPCRs.

In another preferred embodiment, the ABP of the invention binds to human OR10H1 of SEQ ID NO: 2, but does not bind to (or does not detectably bind to) human OR10H1 of SEQ ID NO: 1, SEQ ID NO: 3, SEQ ID NO: 4 or SEQ ID NO: 12; and preferably does not bind to (or does not detectably bind to) any other olfactory receptors and/or human GPCRs.

In another preferred embodiment, the ABP of the invention binds to human OR10H1 of SEQ ID NO: 3, but does not bind to (or does not detectably bind to) human OR10H1 of SEQ ID NO: 1, SEQ ID NO: 2, SEQ ID NO: 4 or SEQ ID NO: 12; and preferably does not bind to (or does not detectably bind to) any other olfactory receptors and/or human GPCRs.

In another preferred embodiment, the ABP of the invention binds to human OR10H1 of SEQ ID NO: 4, but does not bind to (or does not detectably bind to) human OR10H1 of SEQ ID NO: 1, SEQ ID NO: 2, SEQ ID NO: 3 or SEQ ID NO: 12; and preferably does not bind to (or does not detectably bind to) any other olfactory receptors and/or human GPCRs.

In yet another preferred embodiment, the ABP of the invention binds to human OR10H1 of SEQ ID NO: 12, but does not bind to (or does not detectably bind to) human OR10H1 of SEQ ID NO: 1, SEQ ID NO: 2, SEQ ID NO: 3 or SEQ ID NO: 4; and preferably does not bind to (or does not detectably bind to) any other olfactory receptors and/or human GPCRs.

Any of such ABPs of the invention that preferably binds to one or more epitope(s) displayed by one or more extracellular domain(s) of said OR10H1 or variant over any other protein or molecule (such as those above) may, instead or in addition, be described to specifically bind to said OR10H1 or variant.

In some embodiments, the ABP of the invention binds to (e.g. via one or more epitope(s) displayed by one or more extracellular domain(s) of) at least one cynomolgus orthologue of OR10H1, or a variant thereof. In a preferred embodiment, the ABP of the invention binds to (e.g. via one or more extracellular domain(s) of) a cynomolgus orthologue of OR10H1 according to SEQ ID NO: 11, or a variant thereof. In a more preferred embodiment, the ABP of the invention binds to one or more epitope(s) displayed by one or more extracellular domain(s) of human OR10H1 or a variant thereof, and also to (e.g. via one or more epitope(s) displayed by one or more extracellular domain(s) of) a cynomolgus orthologue of human OR10H1, such as one identical to SEQ ID NO: 11, or a variant thereof. Such an ABP of the invention may, despite binding one or more epitope(s) displayed by one or more extracellular domain(s) of both human OR10H1 and a cynomolgus orthologue of human OR10H1, preferentially bind either the human OR10H1 or the cynomolgus orthologue of human OR10H1; i.e., it may specifically bind to one or other of such proteins. In certain of such embodiments, an ABP of the invention specifically binds one or more epitope(s) displayed by one or more extracellular domain(s) of human OR10H1, over its binding to one or more epitope(s) displayed by one or more extracellular domain(s) of a cynomolgus orthologue of human OR10H1.

In those embodiments of the ABP of the invention that binds to an epitope displayed by the 1^{st} extracellular domain (EC) of OR10H1 (e.g., said epitope(s) is/are formed by a stretch of amino acids comprised in amino acid sequence SEQ ID NO: 5 and/or 9), preferred are those such ABPs that also bind to the cynomolgus orthologue of human OR10H1 (SEQ ID NO: 11) (e.g., to the analogous 1^{st} EC domain thereof) more specifically than binding to OR10H5 (e.g. the analogous 1^{st} EC domain thereof, SEQ ID NO: 16) and/or to OR10H2 (e.g. the analogous 1^{st} EC domain thereof, SEQ ID NO: 17).

In those embodiments of the ABP of the invention that binds to an epitope displayed by the 2nd extracellular domain (EC) of OR10H1 (e.g. said epitope(s) is/are formed by a stretch of amino acids comprised in amino acid sequence SEQ ID NO: 6), preferred are those such ABPs that also bind to the cynomolgus orthologue of human OR10H1 (SEQ ID NO: 11) (e.g. to the analogous 2^{nd} EC domain thereof) essentially as specifically as binding to OR10H2 (e.g. the analogous 2^{nd} EC domain thereof, SEQ ID NO: 18) and/or to OR10H5 (e.g. the analogous 2^{nd} EC domain thereof, SEQ ID NO: 19).

In those embodiments of the ABP of the invention that binds to an epitope displayed by the 3^{rd} extracellular domain (EC) of OR10H1 (e.g. said epitope(s) is/are formed by a stretch of amino acids comprised in amino acid sequence SEQ ID NO: 7, 10 and/or 13), preferred are those such ABPs that also bind to OR10H5 (e.g. to the analogous 3^{rd} EC domain thereof, SEQ ID NO: 20) more specifically than binding to the cynomolgus orthologue of human OR10H1 (SEQ ID NO: 11) (e.g. to the analogous 3^{rd} domain thereof) and/or to OR10H2 (e.g. to the analogous 3^{rd} domain thereof, SEQ ID NO: 21).

In those embodiments of the ABP of the invention that binds to an epitope displayed by the 4^{th} extracellular domain (EC) of OR10H1 (said epitope(s) is/are formed by a stretch of amino acids comprised in amino acid sequence SEQ ID NO: 8), preferred are those such ABPs that also bind to the cynomolgus orthologue of human OR10H1 (SEQ ID NO: 11) (e.g., to the 4^{th} domain thereof) more specifically than binding to OR10H5 (e.g., to the analogous 4^{th} EC domain thereof, SEQ ID NO: 22) and/or to OR10H2 (e.g., to the analogous 4^{th} EC domain thereof, SEQ ID NO: 23).

In each of such embodiments, it being understood that the (and as described elsewhere) exact position of EC domains is predicted, and hence a boundary between any given EC domain and its corresponding TM domain of the corresponding orthologue or paralogue of OR10H1 may actually vary by up to several amino acids, such as by 1, 2, 3, 4, 5, 6, 7, 8, 9 or more than 9 amino acids positions, in particular between 1 and about 5 amino acids positions, and preferably 2 or 3 amino acids positions, as given in the preceding paragraphs (and the corresponding sequences).

In some embodiments, the ABP of the invention is an isolated ABP, and/or is one that is substantially pure. Accordingly, in this or other embodiments, the ABP of the invention may be a non-natural ABP, such as a synthetic, modified or recombinant ABP. In particular, an ABP of the invention may contain at least one amino acid substitution (or deletion) modification (such as 2, 3, 4, 5, 6, 7, 8, 9, 10 or more than 10 such modifications, in particular between 1 and about 5 such modifications, preferably 2 or 3 such modifications) relative to a product of nature, such as a human antibody or a rabbit antibody (such as a polyclonal rabbit antibody) or a murine or rat antibody. In another of such embodiments, the ABP of the invention is generated following non-natural immunisation of a (species of) mammal; such as by immunisation with an antigen to which such (species of) mammal is not exposed in nature, and hence will not have naturally raised antibodies against (i.e., it is not found in, or a product of, nature).

In some embodiments, the ABP of the invention will bind to (e.g., via one or more epitope(s) displayed by one or more extracellular domain(s) of) human OR10H1 or a paralogue, orthologue or other variant thereof (such as any OR10H1 or variant described herein) with a K_{D} that is less than 1 nM. In a preferred embodiment, the ABP of the invention will bind (e.g. said epitope(s) of) said OR10H1 or variant with a K_{D} that is less than 100 pM. In a more preferred embodiment, the ABP of the invention will bind said OR10H1 or variant with a K_{D} that is less than 10 pM. In a most preferred embodiment, the ABP of the invention will bind said OR10H1 or variant with a K_{D} that is less than 2 pM. Binding of an ABP of the invention, such as an antibody of the invention, to a human cell line expressing said OR10H1 or variant may, in some embodiments, occur at an EC50 of less than about 10 μg/mL, 5 μg/mL, 2 μg/mL, 1 μg/mL, 0.5 μg/mL or 0.2 μg/mL, preferably with an EC50 of less than 2 μg/mL. Binding of an ABP of the invention, such as an antibody of the invention, to a Cynomolgus cell line expressing an orthologue of said OR10H1 or variant may, in some embodiments, occur at an EC50 of less than about 10 μg/mL, 5 μg/mL, 2 μg/mL, 1 μg/mL, 0.5 μg/mL or 0.2 μg/mL, preferably with an EC50 of less than 2 μg/mL.

Binding affinity is preferably measured by known methods such as titration of antigen binding protein (e.g. antibody) and detection of its binding to antigen expressed on the surface of cells by flow cytometry or by Biacore testing utilizing surface plasmon resonance. A bead based assay is, however, also useful.

More preferably, binding affinity of an antigen binding protein (e.g. an antibody) of the present invention is measured using flow cytometry, for example by using an assay analogous to the method described in Example 6. Briefly, binding of such an ABP to OR10H1-expressing cells (e.g., M579-A2 cells transiently transfected with OR10H1 knock-down siRNA1) is detected by FACS. The ABP may be labelled directly or, such as in the case of an antibody ABP, may be detected using labelled secondary antibody. The signal of cell binding (determined by FACS) obtained from binding of the ABP at various concentrations (such as across a series of dilutions ranging from about 100 μg/mL to 0.1 μg/mL) is used to construct a dose-response curve of binding, from which an EC50 of binding can be readily determined.

As is known in the art, the Biacore system gives formal kinetic values for the binding coefficient between each antigen binding protein (e.g. antibody) and the antigen. Briefly, the Biacore technology uses surface plasmon resonance (SPR) to measure the decay of antigen binding protein from antigen at various concentrations of antigen and at a known concentration of antigen binding protein. For example, chips are loaded with antigen binding protein, washed, and the chip is exposed to a solution of antigen to load the antigen binding proteins with antigen. The chip is then continually washed with a solution without antigen. An initial increase in SPR is seen as the antigen binding protein and antigen complex forms, followed by decay as the antigen-antigen binding protein complex dissociates. This decay in signal is directly proportional to antigen binding protein affinity.

A preferred bead based assay to determine binding affinity is the Luminex (MiraiBio, Inc., Alameda, Calif.) technology. According to this technology, antigen binding proteins (e.g. antibodies) are assayed for how they bind a plurality of different antigen coated beads. In this assay each bead set is preferably coated with a different concentration of antigen. As the Luminex reader has the ability to multiplex all the bead sets, the bead sets are combined and ABP binding to each of the different bead sets is determined. The behaviour of ABPs on the differentially coated beads can then be tracked. Once normalized for ABP concentration, the ABPs which maintain a high degree of binding as one moves from non-antigen limiting concentrations to limited antigen concentrations correlate well to high affinity. Advantageously, these differential shifts can be used to relatively rank ABP affinities. For example, samples with smaller shifts correspond to higher affinity ABPs and samples with larger shifts correspond to lower affinity ABPs.

In some embodiments of the invention, the ABP of the invention will have a Koff rate of <1×10⁻⁵ 1/s. In a preferred embodiment, the ABP of the invention will have a Koff rate of <1×10⁻⁶ 1/s. In a more preferred embodiment, the ABP of the invention will have a Koff rate of <1×10⁻⁷ 1/s. In a most preferred embodiment, the ABP of the invention will have a Koff rate of <1×10⁻⁸ 1/s.

Koff rates are also measurable via surface plasmon resonance using a Biacore system.

ABPs of the invention may compete for the same epitope or an adjacent epitope. Numerous types of competitive binding assays can be used to determine if one ABP competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labelled assay, solid phase direct labelled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labelled RIA (Moldenhauer et al., 1990, Scand. J. Immunol. 32:77-82). Typically, such an assay involves the use of purified antigen bound to a solid surface or cells bearing either of these, an unlabeled test ABP and a labelled reference ABP. Competitive inhibition is measured by determining the amount of label bound to the solid surface or cells in the presence of the test ABP. Usually the test ABP is present in excess. ABPs identified by competition assay (competing ABPs) include ABPs binding to the same epitope as the reference ABPs and ABPs binding to an adjacent epitope sufficiently proximal to the epitope bound by the reference ABP for steric hindrance to occur. Usually, when a competing ABP is present in excess, it will inhibit (e.g., reduce) specific binding of a reference ABP to a common antigen (such as OR10H1 protein or a fragment thereof) by at least 20%, 30%, 40%, 50%, 55%, 60%, 65%, 70%, 75% or more. In some instances, binding is inhibited by at least 80, 85%, 90%, 95%, or 97% or more, in particular between about 20% and 80%, preferably by at least about 30%, and most preferably by at least about 50%.

In some embodiments of the invention, the ABP of the invention comprises the sequences of an antibody heavy chain variable region CDR1, CDR2, and CDR3; and/or the sequences of an antibody light chain variable region CDR1, CDR2, and CDR3. In preferred embodiments the ABP of the invention comprise an antibody heavy chain sequence and/or an antibody light chain sequence, or an antigen binding fragment thereof; wherein the antibody heavy chain sequence, or the fragment thereof, comprises a CDR3 having at least 80% (for 8 amino acid long CDR3 sequences preferably 85%) sequence identity to an amino acid sequence selected from SEQ ID Nos. 35, 43 and 51, and/or wherein antibody light chain sequence, or the fragment thereof, comprises a CDR3 having at least 80% (for 8 amino acid long CDR3 sequences preferably 85%) sequence identity to an amino acid sequence selected from SEQ ID Nos. 39, 47 and 55.

In some embodiments the antibody heavy chain sequence of an ABP of the invention, or the antigen binding fragment thereof, further comprises a CDR1 having at least 80% sequence identity to an amino acid sequence selected from SEQ ID Nos. 33, 41 and 49; and/or a CDR2 having at 80% sequence identity to an amino acid sequence selected from SEQ ID Nos. 34, 42 and 50. In some embodiments the antibody light chain sequence, or the antigen binding fragment thereof, further comprises a CDR1 having at least 80% sequence identity to an amino acid sequence selected from SEQ ID Nos. 37, 45 and 53; and/or a CDR2 having at least 80% sequence identity to an amino acid sequence selected from SEQ ID Nos. 38, 46 and 54. Also in case of CDR1 or CDR2 sequences, the preferred degree of identity may be increased depending on the length of the CDR sequence. Preferred higher degrees of sequence identity include 85%, 90%, and 95%.

In some embodiments the ABP according to the invention comprises an antibody variable chain sequence having at least 80%, preferably 85%, 90%, 95%, or 98% sequence identity to an amino acid sequence selected from SEQ ID Nos. 36, 40, 44, 48, 52 and 56.

In some embodiments the ABP of the invention comprises an antigen binding fragment of an antibody, wherein said antigen binding fragment comprises CDR1, CDR2 and CDR3, preferably which are selected from the CDR1, CDR2 and CDR3 sequences having the respective amino acid sequences of SEQ ID Nos. 33, 34, 35; or 37, 38, 39; or 41, 42, 43; or 45, 46, 47; or 49, 50, 51; or 53, 54, 55; in each case independently, optionally with not more than three or two, preferably one, amino acid substitution(s), insertion(s) and/or deletion(s) compared to these sequences.

In some embodiments of the invention, said CDR1 has an amino acid sequence of SEQ ID No 33, 37, 41, 45, 49 or 53, and CDR2 has an amino acid sequence of SEQ ID No 34, 38, 42, 46, 50 or 54, and CDR3 has an amino acid sequence of SEQ ID No 35, 39, 43, 47, 51 or 55; in each case independently, optionally with not more than three or two, preferably one, amino acid substitution(s), insertion(s) and/or deletion(s) compared to these sequences.

In alternative or additional embodiments of the invention, the ABP is an antibody, or an antigen binding fragment thereof, composed of at least one, preferably two, antibody heavy chain sequences, and at least one, preferably two, antibody light chain sequences, wherein at least one, preferably both, of said antibody heavy chain sequences comprise CDR1 to CDR3 sequences having the amino acid sequences of SEQ ID NO: 33 to 35, and at least one, preferably both, of said antibody light chain sequences comprise CDR1 to CDR3 sequences having the amino acid sequences of SEQ ID NO: 37 to 39; or wherein at least one, preferably both, of said antibody heavy chain sequences comprise CDR1 to CDR3 sequences having the amino acid sequences of SEQ ID NO: 41 to 43, and at least one, preferably both, of said antibody light chain sequences comprise CDR1 to CDR3 sequences having the amino acid sequences of SEQ ID NO: 45 to 47; or wherein at least one, preferably both, of said antibody heavy chain sequences comprise CDR1 to CDR3 sequences having the amino acid sequences of SEQ ID NO: 49 to 51, and at least one, preferably both, of said antibody light chain sequences comprise CDR1 to CDR3 sequences having the amino acid sequences of SEQ ID NO: 53 to 55; in each case of a CDR independently, optionally with not more than three or two, preferably one, amino acid substitution(s), insertion(s) or deletion(s) compared to these sequence.

In alternative or additional embodiments of the invention, an ABP is preferred, wherein the ABP is an antibody, or an antigen binding fragment thereof, composed of at least one, preferably two, antibody heavy chain sequence, and at least one, preferably two, antibody light chain sequence, wherein said antibody heavy chain sequence comprises a variable region having the amino acid sequence of SEQ ID NO: 36, and wherein said antibody light chain sequence comprises a variable region sequence having the amino acid sequence of SEQ ID NO: 40; or wherein said antibody heavy chain sequence comprises a variable region sequence having the amino acid sequence of SEQ ID NO: 44, and wherein said antibody light chain sequence comprises a variable region sequence having the amino acid sequence of SEQ ID NO: 48; or wherein said antibody heavy chain sequence comprises a variable region sequence having the amino acid sequence of SEQ ID NO: 52, and wherein said antibody light chain sequence comprises a variable region sequence having the amino acid sequence of SEQ ID NO: 56; in each case of a variable region sequence independently, optionally with not more than ten, nine, eight, seven, six, five, four, three, two or one, preferably not more than three, amino acid substitutions, insertions or deletions compared to these sequence.

In an additional aspect, the present invention pertains to, and preferred embodiments of other aspects of the invention involve, an ABP that competes for antigen binding to an ABP comprising at least one Complementary Determining Region (CDR) 3 having an amino acid sequence with at least 80% sequence identity to an amino acid sequence selected from SEQ ID NOs. 35, 39, 43, 47, 51 and 55. In particular embodiments, such CDR3-comprising ABP is capable of (specifically) binding to an epitope displayed by one or more extracellular domain(s) of OR10H1 or a paralogue, orthologue or other variant thereof, and in more particular embodiments wherein such epitope(s) and or domain(s) are any of those described elsewhere herein. In further particular of such embodiments, such ABP may be an inhibitor of the expression, function, activity and/or stability of said OR10H1 or variant. In a preferred embodiment, such an ABP competes for binding (such as to an epitope displayed by one or more extracellular domain(s) of OR10H1 protein, or to an epitope displayed by one or more extracellular domain(s) of a paralogue, orthologue or other variant protein thereof) with one or more of the antibodies of the invention described in the example, such competing for binding with 1C3-A1-A1, 1C3-A1-A2 and/or 8A11-B9-A1. In a more preferred embodiment, such an ABP competes for binding (such as to an epitope displayed by one or more extracellular domain(s) of OR10H1 protein, or to an epitope displayed by one or more extracellular domain(s) of a paralogue, orthologue or other variant protein thereof) with the antibody 8A11-H12-E6; and/or with an antibody obtainable from hybridoma Di-8A11-H12-E6 (DSMZ Deposition Number: DSM ACC3310, deposited 26 Oct. 2016).

Numerous types of competitive binding assays can be used to determine if one ABP competes with another, for example: solid phase direct or indirect radioimmunoassay (RIA), solid phase direct or indirect enzyme immunoassay (EIA), sandwich competition assay; solid phase direct biotin-avidin EIA etc. Typically, such an assay involves the use of purified antigen bound to a solid surface or cells bearing either of these, an unlabeled test ABP and a labelled reference ABP. Competitive inhibition is measured by determining the amount of label bound to the solid surface or cells in the presence of the test ABP. Usually the test ABP is present in excess. ABPs identified by competition assay (competing ABPs) include ABP binding to the same epitope as the reference ABP and ABPs binding to an adjacent epitope sufficiently proximal to the epitope bound by the reference ABP for steric hindrance to occur. Usually, when a competing ABP is present in excess, it will inhibit (e.g., reduce) specific binding of a reference ABP to a common antigen (such as an epitope displayed by one or more extracellular domain(s) of OR10H1 protein or a fragment thereof) by at least 20%, 30%, 40%, 50%, 55%, 60%, 65%, 70%, 75% or more. In some instances, binding is inhibited by at least 80, 85%, 90%, 95%, or 97% or more, in particular between about 20% and 80%, preferably by at least about 30%, and more preferably by at least about 50%.

In preferred embodiments of the invention, the ABP (such as one that is an inhibitor or antagonist of expression, function, activity and/or stability of said OR10H1, paralogue, orthologue or other variant thereof) decreases or reduces the resistance of cells (such as tumour cells) that express OR10H1, or the variant of OR10H1, to an immune response. In other certain preferred embodiments of the invention, the ABP (such as one that is an inhibitor or antagonist of expression, function, activity and/or stability of said OR10H1, paralogue, orthologue or other variant thereof) enhances or increases the sensitivity of cells (such as tumour cells) that express OR10H1, or the variant of OR10H1, to an immune response.

The immune response, is, in particular of such embodiments, a cell-mediated immune response such as one mediated by T-cells including cytotoxic T-cells and/or TILs; and/or the immune response is the lysis and/or killing of the cells that express OR10H1 (or a paralogue, orthologue or other variant of OR10H1) that is mediated by cytotoxic T-cells and/or TILs. In other particular of such embodiments, the immune response is a cytotoxic immune response against cells (such as tumour cells) that express OR10H1 (or a paralogue, orthologue or other variant of OR10H1), in particular a cell-mediated cytotoxic immune response such as one mediated by T-cells including cytotoxic T-cells and/or TILs.

Specifically, in certain preferred embodiments the ABP (such as one that is an inhibitor or antagonist of expression, function, activity and/or stability of said OR10H1, paralogue, orthologue or other variant thereof) enhances or increases killing and/or lysis of cells expressing OR10H1, or the variant of OR10H1, (such as tumour cells); preferably killing and/or lysis being mediated by cytotoxic T-cells and/or TILs, and/or mediated by an enhancement of or increase in the sensitivity of the cells expressing OR10H1 (or the variant of OR10H1) to a (cytotoxic) immune response, such an immune response described above, and/or mediated by a decrease in or reduction of the resistance of the cells expressing OR10H1 (or the variant of OR10H1) to a (cytotoxic) immune response, such an immune response described above.

The cells that express OR10H1 (or a paralogue, orthologue or other variant of OR10H1) are, in certain of such preferred embodiments, cancer cells or are cells that originated from a tumor cell. Exemplary cancer or tumor cells can be those as described or exemplified elsewhere herein.

In other certain preferred embodiments of the invention, the ABP (such as one that is an inhibitor or antagonist of expression, function, activity and/or stability of said OR10H1, paralogue, orthologue or other variant thereof) increases T-cell activity and/or survival, which in certain embodiments, may lead to an enhancement of a (cytotoxic) immune response mediated by such T-cells.

In some embodiments the ABP of the invention may comprise in at least one, preferably all, polypeptide chains, antibody constant domain sequences. The origin of the constant domain sequence may be selected from a mouse, rat, donkey, rabbit or human antibody constant domain sequence. The selection of the constant domain is dependent on the intended use of the ABP of the invention. In some embodiments of the invention the ABP is chimerized, optionally is humanized or murinized. Preferred embodiments of the invention pertain to ABP that comprise a rat heavy chain constant domain selected from constant domain sequences of rat IgG 1, 2a, 2b, 2c, and/or rat light chain constant domain kappa A allele or kappa B allele sequences (see Table E3 below). In preferred embodiments of those antibodies of the invention described in the examples, the IgG subclass of the heavy chain is a rat IgG2a or a rat IgG2b (see Table E3 below), and in more preferred of such embodiments, the IgG subclass of the heavy chain of antibody 1C3-A1-A1, 1C3-A1-A2 or 8A11-B9-A1 is rat IgG2b, and the IgG subclass of the heavy chain of antibody 8A11-H12-E6 is rat IgG2a (in each case, see Table E3).

In some embodiments, the ABP of the invention is an antibody. In a preferred embodiment, the ABP of the invention is a monoclonal antibody (mAb). In other embodiments, the ABP is a fragment of an antibody, such as a fragment of a monoclonal antibody.

Antibodies according to the invention (or those from which fragments thereof can be isolated) can include, for instance, chimeric, humanized, (fully) human, or hybrid antibodies with dual or multiple antigen or epitope specificities, antibody fragments and antibody sub-fragments, e.g., Fab, Fab′ or F(ab′)₂ fragments, single chain antibodies and the like (described below), including hybrid fragments of any immunoglobulin or any natural, synthetic or genetically engineered protein that acts like an antibody by binding to a specific antigen to form a complex.

Antibodies according to the invention will typically comprise at least two full-length heavy chains and two full-length light chains, but in some instances may include fewer chains such as antibodies naturally occurring in camelids or sharks, which may comprise only heavy chains. The ABPs, antibodies, or binding fragments may be produced in hybridomas, by recombinant DNA techniques, or by enzymatic or chemical cleavage of intact antibodies.

Accordingly, in some embodiments, the ABP of the invention is a chimeric antibody, such as a human-chimeric antibody.

In other embodiments, the ABP of the invention is an antibody that is, or is derived from, a non-human antibody, such as a non-primate antibody. For example, the antibody can be, or can be derived from, a mouse, rat, rabbit, shark or camelid antibody. In further embodiments, the ABP can comprise CDRs derived from a non-human repertoire of CDRs, or that comprises at least one CDR that includes at least one amino acid substitution modification relative to a human CDR; and/or can comprise a variant Fc domain.

In some of such embodiments, the ABP is not the anti-OR10H1 antibodies ab107628 and/or ab137315 (Abcam, UK), and/or is not a polyclonal rabbit antibody, such as is not any anti-OR10H1 antibody listed on www.antibodypedia.com/gene/54315/OR10H1 on the date of filing of the present invention.

In a preferred embodiment, the ABP of the invention is a humanised antibody. In a more preferred embodiment, the ABP of the invention is a human antibody. In one most preferred embodiment, the ABP of the invention is a human monoclonal antibody; and in another most preferred embodiment the ABP of the invention is a fragment of human antibody, such as a fragment of a human monoclonal antibody.

Monoclonal antibodies in accordance with the present invention may be prepared by methods well known to those skilled in the art. For example, mice, rats or rabbits may be immunized with an antigen of interest together with adjuvant. Splenocytes are harvested as a pool from the animals that were administered 3 immunizations at 2-week intervals with test bleeds performed on alternate weeks for serum antibody titers. Splenocytes are prepared as 3 aliquots that are either used immediately in fusion experiments or stored in liquid nitrogen for use in future fusions. Fusion experiments are then performed according to the procedure of Stewart & Fuller, J. Immunol. Methods 1989, 123:45-53.

Supernatants from wells with growing hybrids are screened by enzyme-linked immunosorbent assay (ELISA) for mAb secretors on 96-well ELISA plates coated with the antigen. ELISA-positive cultures are cloned by limiting dilutions, typically resulting in hybridomas established from single colonies after 2 serial cloning experiments. The ability of an antibody, including an antibody fragment or sub-fragment, to bind to a specific antigen can be determined by binding assays known in the art, for example, using the antigen of interest as the binding partner.

The antibodies described herein may alternatively be prepared through the utilization of the XenoMouse® technology. Such mice are capable of producing human immunoglobulin molecules and antibodies and are deficient in the production of murine immunoglobulin molecules and antibodies. In particular, a preferred embodiment of transgenic production of mice and antibodies is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000. See also Mendez et al., Nature Genetics, 15:146-156 (1997). Through the use of such technology, fully human monoclonal antibodies to a variety of antigens have been produced. Essentially, XenoMouse® lines of mice are immunized with an antigen of interest (e.g. OR10H1), lymphatic cells (such as B-cells) are recovered from the hyper-immunized mice, and the recovered lymphocytes are fused with a myeloid-type cell line to prepare immortal hybridoma cell lines. These hybridoma cell lines are screened and selected to identify hybridoma cell lines that produce antibodies specific to the antigen of interest.

The antibodies described herein may be prepared by genetic immunization methods in which native proteins are expressed in vivo with normal post-transcriptional modifications, avoiding antigen isolation or synthesis. For example, hydrodynamic tail or limb vein delivery of naked plasmid DNA expression vectors can be used to produce the antigen of interest in vivo in mice, rats, and rabbits and thereby induce antigen-specific antibodies (Tang et al, Nature 356(6365): 152-4 (1992); Tighe et al, Immunol. Today 19(2) 89-97 (1998); Bates et al, Biotechniques, 40(2) 199-208 (2006); Aldevron-Genovac, Freiburg, Del.). This allows the efficient generation of high-titre, antigen-specific antibodies which may be particularly useful for diagnostic and/or research purposes.

A variety of gene delivery methods can be used, including direct injection of naked plasmid DNA into skeletal muscle, lymph nodes, or the dermis, electroporation, ballistic (gene gun) delivery, and viral vector delivery.

Human antibodies can also be derived by in vitro methods. Suitable examples include but are not limited to phage display (CAT, Morphosys, Dyax, Biosite/Medarex, Xoma, Symphogen, Alexion, Affimed) and the like. In phage display, a polynucleotide encoding a single Fab or Fv antibody fragment is expressed on the surface of a phage particle (see e.g., Hoogenboom et al., J. Mol. Biol., 227: 381 (1991); Marks et al., J Mol Biol 222: 581 (1991); U.S. Pat. No. 5,885,793). Phage are “screened” to identify those antibody fragments having affinity for target. Thus, certain such processes mimic immune selection through the display of antibody fragment repertoires on the surface of filamentous bacteriophage, and subsequent selection of phage by their binding to target. In certain such procedures, high affinity functional neutralizing antibody fragments are isolated. A complete repertoire of human antibody genes may thus be created by cloning naturally rearranged human V genes from peripheral blood lymphocytes (see, e.g., Mullinax et al., Proc Natl Acad Sci (USA), 87: 8095-8099 (1990)).

Where it is desired to improve the affinity of antibodies according to the invention, this can be achieved by a number of affinity maturation protocols including maintaining the CDRs (Yang et al., J. Mol. Biol., 254, 392-403, 1995), chain shuffling (Marks et al., Bio/Technology, 10, 779-783, 1992), use of mutation strains of E. coli. (Low et al., J. Mol. Biol., 250, 350-368, 1996), DNA shuffling (Patten et al., Curr. Opin. Biotechnol., 8, 724-733, 1997), phage display (Thompson et al., J. Mol. Biol., 256, 7-88, 1996) and PCR (Crameri, et al., Nature, 391, 288-291, 1998). All of these methods of affinity maturation are discussed by Vaughan et al. (Nature Biotechnology, 16, 535-539, 1998).

Light chains of human antibodies generally are classified as kappa and lambda light chains, and each of these contains one variable region and one constant domain. Heavy chains are typically classified as mu, delta, gamma, alpha, or epsilon chains, and these define the antibody's isotype as IgM, IgD, IgG, IgA, and IgE, respectively. IgG has several subtypes, including, but not limited to, IgG1, IgG2, IgG3, and IgG4. IgM subtypes include IgM, and IgM2. IgA subtypes include IgA1 and IgA2. In humans, the IgA and IgD isotypes contain four heavy chains and four light chains; the IgG and IgE isotypes contain two heavy chains and two light chains; and the IgM isotype contains five heavy chains and five light chains. Antibodies according to the invention may be IgG, IgE, IgD, IgA, or IgM immunoglobulins.

Accordingly, in some embodiments, the ABP of the invention is an IgG antibody or fragment thereof. In some embodiments, the ABP of the invention is an IgE antibody or fragment thereof. In some embodiments, the ABP of the invention is an IgD antibody or fragment thereof. In some embodiments, the ABP of the invention is an IgA antibody or fragment thereof. In some embodiments, the ABP of the invention is an IgM antibody or fragment thereof. Preferably the ABP of the invention is, comprises or is derived from an IgG immunoglobulin or fragment thereof; such as a human, human-derived IgG immunoglobulin, or a rabbit- or rat-derived IgG, and/or an IgG2 immunoglobulin, or fragment thereof. When the ABP of the invention is, comprises or is derived from a rat-derived IgG, then preferably, the ABP is, comprises or is derived from, a rat IgG2a or IgG2b immunoglobulin. When the ABP of the invention is, comprises or is derived from a human-derived IgG, then more preferably, the ABP of the invention is, comprises or is derived from a human IgG1, IgG2 or IgG4, most preferably, the ABP of the invention is, comprises or is derived from a human IgG1 or IgG2.

Antibody fragments include “Fab fragments”, which are composed of one constant and one variable domain of each of the heavy and the light chains, held together by the adjacent constant region of the light chain and the first constant domain (CH1) of the heavy chain. These may be formed by protease digestion, e.g. with papain, from conventional antibodies, but similar Fab fragments may also be produced by genetic engineering. Fab fragments include “Fab-SH”, which are Fab fragments containing at least one free sulfhydryl group.

Fab′ fragments differ from Fab fragments in that they contain additional residues at the carboxy terminus of the first constant domain of the heavy chain including one or more cysteines from the antibody hinge region. Fab′ fragments include “Fab′-SH”, which are Fab′ fragments containing at least one free sulfhydryl group.

Further, antibody fragments include F(ab′)₂ fragments, which contain two light chains and two heavy chains containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains. F(ab′)₂ fragments may be prepared from conventional antibodies by proteolytic cleavage with an enzyme that cleaves below the hinge region, e.g. with pepsin, or by genetic engineering.

An “Fv region” comprises the variable regions from both the heavy and light chains, but lacks the constant regions. “Single-chain antibodies” or “scFv” are Fv molecules in which the heavy and light chain variable regions have been connected by a flexible linker to form a single polypeptide chain, which forms an antigen binding region.

An “Fc region” comprises two heavy chain fragments comprising the CH2 and CH3 domains of an antibody. The two heavy chain fragments are held together by two or more disulphide bonds and by hydrophobic interactions of the CH3 domains.

Accordingly, in some embodiments, the ABP of the invention is an antibody fragment selected from the list consisting of: Fab, Fab′-SH, Fv, scFv and F(ab′)₂.

In those embodiments of ABPs that are fragments of immunoglobulins, such as an antibody fragment, preferred are those fragments capable of binding to an epitope displayed by one or more extracellular domain(s) of OR10H1, or a paralogue, orthologue or other variant thereof, such as any epitope or other binding characteristic as described herein: and more preferably said fragment is a modulator (such as an inhibitor or antagonist) of the expression, function, activity and/or stability of OR10H1 or a paralogue, orthologue or other variant of OR10H1.

In a preferred embodiment, the ABP of the invention is an antibody wherein at least a portion of the framework sequence of said antibody or fragment thereof is a human consensus framework sequence.

In some embodiments, the ABP is modified or engineered to increase antibody-dependent cellular cytotoxicity (ADCC). As will now be understood by the person of ordinary skill, such ABPs of the invention will have particular utility in the therapy of diseases or disorders associated with cellular resistance against immune cells like CTLs (such as an OR10H1-positive cancer); as the ADCC mechanism (a cell-mediated immune defense whereby an effector cell of the immune system actively lyses a target cell, whose membrane-surface antigens have been bound by specific antibodies) would be enhanced in respect of the cells having resistance against immune cells like CTLs, hence leading to an increase in attachment by and/or lysis of such cells by effector cells of the immune system.

Various techniques to modify or engineer an ABP of the invention to increase ADCC are known (Satoh et al, 2006; Expert Opin Biol Ther 6:1161; WO2009/135181), and hence such embodiments include those wherein an ABP of the invention may be afucosylated (GlycArt Biotechnology) e.g., in which antibodies are produced in CHO cells in which the endogenous FUT8 gene has been knocked out; or the ABP may be a “Sugar-Engineered Antibody” (Seattle Genetics), e.g. in which fucose analogues are added to antibody-expressing CHO cells, resulting in a significant reduction in fucosylation.

In some embodiments, the ABP of the invention binds to OR10H1, or a paralogue, orthologue or other variant thereof, wherein said ABP binds to two or more epitope(s) displayed by one or more extracellular domain(s) of said OR10H1, paralogue, orthologue or other variant, e.g., when expressed on the surface of a mammalian cell.

In some embodiments, the ABP of the invention binds to (an) epitope(s) which is/are displayed by two or more extracellular domains of OR10H1 or a paralogue, orthologue or other variant thereof.

As described above, in one aspect the invention provides ABPs that bind to one or more epitope(s) displayed by one or more extracellular domain(s) of human OR10H1, or a paralogue, orthologue or other variant thereof. Such extracellular domain(s) can include any extracellular domain represented by: SEQ ID NO:5, SEQ ID NO:6, SEQ ID NO:7, SEQ ID NO:8, SEQ ID NO:9; SEQ ID NO:10 and/or SEQ ID NO:13.

In some embodiments, the epitope(s) displayed by one or more extracellular domain(s) of said OR10H1, paralogue, orthologue or other variant is/are formed by a stretch of amino acids from about 3 amino acids (such as from 4 or from 5 amino acids) up to the maximum number of amino acids in the respective extracellular domain, which correspond to SEQ ID NO:5, SEQ ID NO:6, SEQ ID NO:7, SEQ ID NO:8, SEQ ID NO:9, SEQ ID NO:10 and/or SEQ ID NO:13. For example, the epitope(s) is/are formed by a stretch of amino acids of between about 3 and 37 amino acids, of between about 3 and 25 amino acids, of between about 3 and 24 amino acids, of between about 5 and 20 amino acids, of between about 3 and 13 amino acids, of between about 5 and 13 amino acids or of between about 5 and 10 amino acids comprised in any of the amino acid sequences independently selected from the group consisting of: SEQ ID NO:5, SEQ ID NO:6, SEQ ID NO:7, SEQ ID NO:8, SEQ ID NO:9, SEQ ID NO:10 and/or SEQ ID NO:13. Preferably for SEQ ID NO:5, the epitope(s) is/are formed by a stretch of amino acids of between 5 and 20 amino acids, between 5 and 10 amino acids or between 4 and 8 amino acids, comprised in the amino acid sequence of SEQ ID NO:5. Preferably for SEQ ID NO:6, the epitope(s) is/are formed by a stretch of amino acids of between 5 and 20 amino acids, between 5 and 10 amino acids or between 4 and 8 amino acids, comprised in the amino acid sequence of SEQ ID NO:6. Preferably for SEQ ID NO:7, the epitope(s) is/are formed by a stretch of amino acids of between 5 and 20 amino acids, between 5 and 10 amino acids or between 4 and 8 amino acids, comprised in the amino acid sequence of SEQ ID NO:7. Preferably for SEQ ID NO:8, the epitope(s) is/are formed by a stretch of amino acids of between 5 and 20 amino acids, between 5 and 10 amino acids or between 4 and 8 amino acids, comprised in the amino acid sequence of SEQ ID NO:8. Preferably for SEQ ID NO:9, the epitope(s) is/are formed by a stretch of amino acids of between 5 and 20 amino acids, between 5 and 10 amino acids or between 4 and 8 amino acids, comprised in the amino acid sequence of SEQ ID NO:9. Preferably for SEQ ID NO:10, the epitope(s) is/are formed by a stretch of amino acids of between 5 and 20 amino acids, between 5 and 10 amino acids or between 4 and 8 amino acids, comprised in the amino acid sequence of SEQ ID NO:10. Preferably for SEQ ID NO:13, the epitope(s) is/are formed by a stretch of amino acids of between 5 and 20 amino acids, between 5 and 10 amino acids or between 4 and 8 amino acids, comprised in the amino acid sequence of SEQ ID NO:13.

In some embodiments, the ABP of the invention binds to OR10H1, or a paralogue, orthologue or other variant thereof, wherein said ABP binds to one or more epitope(s) displayed by one or more extracellular domain(s) of said OR10H1, paralogue, orthologue or other variant, wherein each of said extracellular domain(s) is independently selected from the group consisting of: SEQ ID NO:5, SEQ ID NO:6, SEQ ID NO:7, and SEQ ID NO:8.

In some embodiments, the ABP of the invention binds to OR10H1, or a paralogue, orthologue or other variant thereof, wherein said ABP binds to one or more epitope(s) displayed by one or more extracellular domain(s) of said OR10H1, paralogue, orthologue or other variant, wherein each of said extracellular domain(s) is independently selected from the group consisting of: SEQ ID NO:9, SEQ ID NO:6, SEQ ID NO:7, and SEQ ID NO:8.

In some embodiments, the ABP of the invention binds to OR10H1, or a paralogue, orthologue or other variant thereof, wherein said ABP binds to one or more epitope(s) displayed by one or more extracellular domain(s) of said OR10H1, paralogue, orthologue or other variant, wherein each of said extracellular domain(s) is independently selected from the group consisting of: SEQ ID NO:5, SEQ ID NO:6, SEQ ID NO:10, and SEQ ID NO:8.

In some embodiments, the ABP of the invention binds to OR10H1, or a paralogue, orthologue or other variant thereof, wherein said ABP binds to one or more epitope(s) displayed by one or more extracellular domain(s) of said OR10H1, paralogue, orthologue or other variant, wherein each of said extracellular domain(s) is independently selected from the group consisting of: SEQ ID NO:5, SEQ ID NO:6, SEQ ID NO:8, and SEQ ID NO:13.

In some embodiments, the ABP of the invention is variant specific, i.e., it binds to one of the wild-type (WT), G16R, G16R/A65V, A167T or H175Q variants of OR10H1, but not to the other OR10H1 variants.

In a preferred embodiment, the ABP of the invention binds to OR10H1, or a paralogue, orthologue or other variant thereof, wherein said ABP binds to one or more epitope(s) displayed by one or more extracellular domain(s) of said OR10H1, paralogue, orthologue or other variant when expressed on the surface of a mammalian cell, wherein at least one of said epitopes is formed by a stretch of amino acids of between about 3 and 37 amino acids, such as between 5 and 20 amino acids, between 5 and 10 amino acids or between 4 and 8 amino acids, comprised in the amino acid sequence of SEQ ID NO:6.

Epitopes on the extracellular domains of OR10H1 may be linear or conformational. The epitope sequence can be determined by antigen mutation studies, wherein amino acids in the antigen (e.g. in extracellular domains of OR10H1) are sequentially mutated and the binding of a (optionally labelled) reference ABP to the mutated antigen is compared to the binding to the wild-type (non-mutated) antigen. The abolition or reduction of binding identifies amino acids that comprise an epitope. Such antigen mutation studies can be performed by high-throughput mutagenesis mapping utilizing a comprehensive mutation library containing a unique amino acid mutation (conservative, non-conservative, or alanine) at every potential epitope position.

By using CLIPS™ (Chemical Linkage of Peptides onto Scaffolds) technology, conformational peptide libraries are created for detecting conformational, discontinuous, and complex epitopes on oligomeric proteins. CLIPS™ Precision Epitope Mapping uses arrays with large, surface-immobilized libraries of conformationally CLIPS-constrained peptides derived from the target protein. The binding of the antibody to each peptide construct of the entire library is determined. This affinity information is used in iterative screens to define the sequence and conformation of epitopes in detail.

In certain embodiments, definitive delineation of a CDR and identification of residues comprising the binding site of an ABP is accomplished by solving the structure of the ABP and/or solving the structure of the ABP-receptor complex. In certain embodiments, that can be accomplished by any of a variety of techniques known to those skilled in the art, such as X-ray crystallography—see for example, Harlow and Lane Antibodies: A Laboratory Manual Cold Spring Harbor Laboratory Press, Cold Spring Harbor, N.Y. (1990) and Smyth and Martin, X Ray Crystallography, Mol. Pathol. 53(1) 8-14 (2000).

Another technique for mapping antibody epitopes is to use genetically encoded photoactivatable cross-linkers. In this technique, the protein of interest, such as a GPCR, has a photolabile unnatural amino acid (e.g. pazido-L-phenylalanine (azF)) incorporated at various positions within an extracellular domain. Interactions between monoclonal antibodies and the labelled-protein (e.g. azF-protein) are then mapped using targeted loss-of-function studies and photo cross-linking in whole cells. An ELISA-based assay can be used to quantitate cross-linking efficiency and determine which residues of the extracellular domain the monoclonal antibodies cross-link—see Ray-Saha et al, Biochemistry 53, 1302-1310 (2014). These data can also be mapped to a crystal structure of the protein. Such a targeted photo-cross-linking method is complementary to loss-of-function mutagenesis and can be used for studying mAbs with discontinuous epitopes on OR10H1 or a variant thereof.

The epitope to which the ABPs of the invention bind can be further investigated by various approaches, which as well as those methods described in the Examples may additionally or alternatively include:

Epitope binning: Epitope binning using competition between pair-wise analysis of ABPs is used to determine the number of different epitopes on said OR10H1 or variant to which an ABP panel binds, thus providing an indication of diversity (e.g., analogously to that described by Hutchings et al, 2014; MAbs 6:246).

Epitope mapping: A. Sets of linear peptides representing, or constrained peptides mimicking the 3-dimensional structure of, said OR10H1 or variant extracellular domain(s) and collectively covering the extracellular loops as well as the N-terminus are designed and synthesized. The ABPs of the invention are tested for binding to these peptides (e.g., analogously to that described by Larsson et al, 2001; J Immunol Method 370:14; Hutchings et al, 2014; Boshuizen et al, 2014; MAbs. 6:1415; Rossant et al, 2014; MAbs. 6:1425). B. Epitope mapping is performed using a series of artificial fusion proteins that carry peptides derived from said OR10H1 or variant extracellular regions. The ABPs are tested for binding to these fusion proteins (e.g., analogously to that described by Sangawa et al, 2008; Hybridoma. 27:331). C. A library of mutations covering a broad set of amino acids in said OR10H1 or variant is generated to identify amino acid changes that resulted in loss of ABP reactivity (e.g., analogously to that described by Paes Cet al, 2009; J Am Chem Soc. 131:6952). D. Epitopes recognized by ABPs are found by selection of peptides from random peptide libraries displayed on phage. A computer algorithm is used to map the epitopes to discontinuous segments of said OR10H1 or variant that are distant in the primary sequence but are in close spatial proximity in the structure (e.g., analogously to that described by Bailey et al, 2003; Protein Sci. 12:2453). E. The regions harbouring loops of said OR10H1 or variant are exchanged for homologous fragments either from a related GPCR or from a species orthologue. The ABPs are assessed for binding to these chimeric proteins (e.g., analogously to that described by Chamorro et al, 2014; MAbs. 6:1000; Takeda et al, 2015; Sci Rep 5:1133). F. Co-crystallization of ABP to said OR10H1 or variant GPCR (e.g., analogously to that described by Rasmussen et al, 2011; Nature. 469:175; Ring et al, 2013; Nature. 502:575).

An ABP of the present invention may be mono-specific (i.e, it possesses antigen binding domain(s) that bind to only one antigen) or may be multi-specific (i.e, it possesses two or more different antigen binding domain(s) that bind to different antigens). For example, a “bi-specific”, “dual-specific” or “bifunctional” ABP or antibody is a hybrid ABP or antibody, respectively, having two different antigen binding sites. Bi-specific antigen binding proteins and antibodies are a species of multi-specific antigen binding protein antibody and can be produced by a variety of methods including, but not limited to, fusion of hybridomas or linking of Fab′ fragments (see, e.g., Songsivilai and Lachmann, 1990; Kostelny et al., 1992). The two binding sites of a bi-specific antigen binding protein or antibody will bind to two different epitopes, which can reside on the same or different protein targets.

Accordingly, in some embodiments, the ABP of the invention binds (e.g. via one or more first antigen binding domain(s)) to one or more epitope(s) displayed by one or more extracellular domain(s) of said OR10H1, paralogue, orthologue or other variant when expressed on the surface of a mammalian cell, and in addition comprises one or more additional antigen binding domain(s) that bind(s) to antigen(s) other than said OR10H1 or variant. Such other antigen may, in certain embodiments of the inventive ABP, be another olfactory receptor or may be another GPCR; and/or such other antigen may be an antigen present on a mammalian T-cell. Antigens present on a mammalian T-cell, that may be bound by such an additional antigen binding domain, include CD3, CD40, OX-40, ICOS and 4-1BB.

Such other antigen may, in certain embodiments of the inventive ABP, also be albumin, e.g., human albumin. It may also be another component of blood or blood serum the binding of which by the ABP will confer an extended serum half-life upon the ABP, e.g., a half-life similar to that when bound to albumin.

In particular embodiments, the ABP of the invention is a “bi-specific” ABP, such as a bi-specific antibody.

In certain embodiments, as a consequence of binding to epitope(s) displayed by one or more extracellular domain(s) of human OR10H1 (or a paralogue, orthologue or other variant thereof), the antigen binding proteins provided herein can impact one or more characteristics, properties and/or abilities of said OR10H1 or variant, such as to change, modify or alter (e.g., to inhibit/antagonise) one or more of expression, function, activity and/or stability of said OR10H1 or variant. As will be apparent, such expression, function, activity and/or stability of said OR10H1 or variant will be, typically, modulated (or investigated/measured) when it is present (or to be present) on the surface of a mammalian cell. However, expression, function, activity and/or stability of said OR10H1 or variant may be, by use of appropriate in-vitro technologies, modulated (or investigated/measured) when it is not present or associated with the surface or membrane of a mammalian cell, for example in in-vitro studies or assays.

Accordingly, in some embodiments, the ABP of the invention is a modulator of the expression, function, activity and/or stability of said OR10H1 or variant, such as when present on the surface of a mammalian cell.

For example, inhibitor or antagonist ABPs provided herein may, in certain embodiments, reduce the expression, function, activity and/or stability of said OR10H1 or variant by about 30%, 50%, 60%, 70%, 80%, 90%, 95%, 96%, 97% 98%, 99% or more, preferably by at least about 50%, such as may be determined by a chromium release assay analogous to that as described in Example 1. For example, measuring/comparing such expression, function, activity and/or stability of said OR10H1 or variant can be determined in an assay as described herein.

In some embodiments, the ABP of the invention is an inhibitor or antagonist of expression, function, activity and/or stability of said OR10H1 or variant, such as when present on the surface of a mammalian cell.

In a preferred embodiment, the ABP of the invention is an inhibitor (or antagonist) of the function and/or activity of said OR10H1 or variant, such as when present on the surface of a mammalian cell.

In certain of such embodiments of the invention, the ABP of the invention that binds to one or more epitope(s) displayed by one or more extracellular domain(s) of OR10H1, or a paralogue, orthologue or other variant thereof will inhibit said OR10H1 or variant function and/or activity with an IC50 of less than about 100 μM, 10 μM, 1 μM, 100 nM or 10 nM, preferably of less than about 1 μM. In another preferred such embodiment, the ABP of the invention will inhibit said OR10H1 or variant function and/or activity with an IC50 of less than 1 nM. In a more preferred such embodiment, the ABP of the invention will inhibit said OR10H1 or variant function and/or activity with an IC50 of less than 500 pM. In a most preferred such embodiment, the ABP of the invention will inhibit said OR10H1 or variant function and/or activity with an IC50 of less than 100 pM. Binding of an ABP may be determined and measured by way of an EC50, using methods as described elsewhere herein.

The IC50 of an inhibitor/antagonist ABP can be determined by examining the effect of increasing concentrations of the inhibitor/antagonist ABP on the function and/or activity being investigated as the biological response (for example, an inhibition of said OR10H1 that results in and/or is measured by enhancement of a cell-mediated immune response and/or an increase in immune cell activity and/or survival), from a maximum such response. Responses are then normalized to the maximum and plotted against the log concentration of inhibitor/antagonist ABP in order to construct a dose-response curve, from which the concentration can be determined that gives 50% inhibition of the maximum biological response. Suitable assays for such function and/or activity are described elsewhere herein, and include for example a chromium-release cytotoxicity assay, such as described in Example 1.

In some embodiments, when the ABP of the invention acts as an inhibitor or antagonist of expression, function, activity and/or stability of said OR10H1 or variant, it reduces the amount and/or surface concentration of said OR10H1 or variant present on the surface of a mammalian cell. For example, in one preferred example, an inhibiting or antagonistic ABP of the invention may induce internalisation, and optionally degradation, of protein of said OR10H1 or variant from the surface of a mammalian cell, such as via a mechanism similar to that described for EGFR (Henriksen et al, 2013; PLoS ONE 8(3): e58148) or ErbB3 receptor (Belleudi et al, 2012; Cell Cycle 11:1455).

In other embodiments, when the ABP of the invention acts as an inhibitor or antagonist of expression, function, activity and/or stability of said OR10H1 or variant, it modulates the binding of one or more chemostimuli of OR10H1 to OR10H1 expressed on the surface of a mammalian cell. Such a chemostimulus may be a ligand expressed and/or secreted by an immune cell (such as a T-cell), or one displayed on the surface of said immune cell. A ligand secreted by such an immune cell may, in some embodiments, be a metabolite of said immune cell. Without being bound by theory, such an immune-cell derived chemostimulus may be specific or characteristic or one or more type of immune cells (such as T-cells); and/or may—except when blocked by an ABP of the invention—bind to OR10H1 (or the variant) and trigger the olfactory receptor signaling mechanism, associated with Cx32-mediated transport of cAMP when the immune cell is bound to the tumour cell, and ultimately associated with a “shut-down” of the immune cell's normal cytotoxic activity against the tumour cell (e.g. by decrease in activity or cell death of such immune cell).

In yet other embodiments, when the ABP of the invention acts as an inhibitor or antagonist of expression, function, activity and/or stability of said OR10H1 or variant, it modulates the binding of immune cells (such as T-cells) to the surface of a mammalian cell expressing said OR10H1 or variant, such as on its surface. In further embodiments, when the ABP of the invention acts as an inhibitor or antagonist of expression, function, activity and/or stability of said OR10H1 or variant, it increases the activity of immune cells, such as T-cells (for example when bound to the surface of a mammalian cell expressing said OR10H1 or variant, such as on its surface).

In certain embodiments, an ABP of the invention reduces the amount and/or surface concentration of said OR10H1 or variant present on the surface of a mammalian cell; and/or it modulates the binding of T-cells; and/or it increases the activity of T-cells (for example, when bound to the surface of a mammalian cell). Methods to determine such amount and/or surface concentration, or binding and/or activity of T-cells are known to the person of ordinary skill, or can be found disclosed elsewhere herein. In particular, the assay(s) described in Example 1, 2 and 4 may be used to determine the activity of T-cells.

In some embodiments, when the ABP of the invention acts as an inhibitor or antagonist of expression, function, activity and/or stability of said OR10H1 or variant, it enhances a cell-mediated immune response, such as that mediated by an activated cytotoxic T-cell (CTL), to a mammalian cell expressing said OR10H1 or variant, such as on its surface; and/or it increases immune cell (such as T-cell, and in particular CTLs and/or TILs) activity and/or survival in the presence of a mammalian cell expressing said OR10H1 or variant, such as on its surface. Methods to determine such cell-mediated immune response, or activity and/or survival of T-cells are known to the person of ordinary skill, or can be found disclosed elsewhere herein. In particular, the assay(s) described in Example 2 may be used to determine the survival of T-cells.

It will now be apparent to the person of ordinary skill, given the present invention is related to the surprising finding of OR10H1-mediated resistance to one or more immune responses, that an “enhancement” or an “increase” in immune response (such as a cell-based immune response, in particular an increase in T-cell activity and/or survival), may be an increase or enhancement relative to that mediated (e.g. reduced) by this newly described OR10H1 activity (e.g. such OR10H1-mediated immune resistance). Accordingly, the “enhancement” or “increase” (and like terms) may equally be considered a “release” of such OR10H1-mediated reduction of an immune response (and reflected by the resistance to the immune response), such that the immune response resulting from inhibition of OR10H1 (or of the variant of OR10H1) expression, function and/or stability, may be so enhanced or increased relative to that in the cells and/or subject prior to such inhibition, but the immune response may also be considered to be at a level (after such inhibition) equivalent to, or essentially the same as, that typically present in a healthy subject or with normal cells. By way of one limiting specific example, an ABP of the present invention while may be described as “enhancing” activity and/or survival of T-cells, it may also be described as “preventing a reduction” in such activity and/or survival.

One particular method to assay a cell-mediated immune response to a mammalian cell expressing said OR10H1 or variant, includes assays to determine the cytotoxicity of said mammalian cells (such as tumour or cancer cells expressing said OR10H1 or variant) cultured in the presence of the ABP and immune cells.

For example, a chromium-release cytotoxicity assay allows one to determine how effectively lymphocytes (e.g. T cells) kill tumour cells. A chromium-release cytotoxicity assay may be performed analogously to the one described in WO 2015/028515. For example: Tumour cells are treated (e.g. in 25 cm² culture flasks) with an ABP of the invention or alternatively transfected with a NAC (e.g. a vector) encoding an ABP of the invention, using a transfection reagent as per manufacturers protocol. An ABP that does not bind OR10H1 or an NAC encoding an ABP that does not bind OR10H1 may be used as negative controls. After a period of incubation (e.g. 72 hours), the tumour cells are harvested, washed and labelled with radioactive chromium (⁵¹Cr) which has the property of binding to the cellular proteins of cultured cells (e.g. 200 μl ⁵¹Cr [Perkin-Elmer, Germany] per 10⁶ target cells for around 45 min at 37° C.). For measuring antibody blockade of OR10H1, tumour cells are first incubated with of blocking antibody (e.g. 30 μg/ml for around 30 minutes) on ice and then washed and labelled with ⁵¹Cr. After labelling, the tumour cells are carefully washed to remove cell-free chromium and they are then co-cultured (e.g. 3000 tumour cells/well) with T cells for a period of time at a T cell to tumour cell ratio of 1:1 to 100:1 (e.g. in 96 well plates for around 4 hours at 37° C.). The plates are then spun down and the supernatant is harvested for measuring the radioactivity released by dead tumour cells using a Gamma counter (e.g. Cobra counter Packard, Perkin Elmer, Rodgau, Germany). The amount of radioactivity which is released in the supernatant is taken as an indicator of the amount of lysis which has occurred. This assay takes advantage of the fact that cytotoxic T-cells kill their target cells by disrupting the integrity of the cell membrane thereby allowing the release of ⁵¹Cr bound to protein from the target cell. A simple calculation of the amount of cell bound ⁵¹Cr versus free ⁵¹Cr allows one to quantify the amount of cellular cytotoxicity. Tumour cells that can be used with this assay are, for example, M579-A2 cells, SW480 cells, M615 cells.

Polyclonal CD8 T cells from healthy donors can be used as effector cells for this assay. To this end, CD8+ T cells can be isolated from peripheral blood mononuclear cells (PBMCs) of healthy donors and activated using, for example, anti-CD3/anti-CD28 activation beads. Subsequently, these activated CD8 T cells are co-cultured with radioactively labelled tumour cells treated with or without an ABP of the invention as above, with the additional presence of anti-CD3×anti EpCAM bi-specific antibody or anti-CD3×anti-CD19 control bi-specific antibody (e.g. each at 5 μg/ml). As a control for spontaneous release, the labelled cells can be co-incubated with media alone; for maximum release, the cells can be incubated with 10% Triton X-100 instead of T cells. The % specific lysis is then calculated by the formula given below:

% specific lysis=(Experimental release−Spontaneous release)/(Maximum release−Spontaneous release)×100

In some embodiments, when the ABP of the invention acts as an inhibitor or antagonist of expression, function, activity and/or stability of said OR10H1 or variant, it decreases cAMP response element-binding protein (CREB) phosphorylation in immune cells (such as T-cells, and in particular CTLs and/or TILs); and/or it activates lymphocyte-specific protein tyrosine kinase (Lck), for example by reduced phosphorylation of the LcK Tyr505 domain in such immune cells. Methods to determine such phosphorylation in immune cells (such as T-cells) are known to the person of ordinary skill, or can be found disclosed elsewhere herein. In particular, the assay(s) described in Example 4 may be used to determine the phosphorylation of CREB and/or Lck in T-cells.

In some embodiments, when the ABP of the invention acts as an inhibitor or antagonist of expression, function, activity and/or stability of said OR10H1 or variant, it may reduce phosphorylation of protein kinase A (PKA) in said immune cells and in particular CTLs and/or TILs (such as T-cells).

In further embodiments, when the ABP of the invention acts as an inhibitor or antagonist of expression, function, activity and/or stability of said OR10H1 or variant, it may reduce the level of cAMP in said immune cells. In certain of such embodiments, said reduction of cAMP is associated with a reduction in the transport of cAMP into said immune cells from one or more other cells (such as a tumour cell expressing said OR10H1 or variant) via a gap-junction protein, for example connexin 32 (Cx32), expressed by said other cells.

The term “associated with”, in the context of this and other embodiments can mean that two variables, effects or phenotypes are correlated to each other, that they are related to each other, or that there is some kind of causative link between a first variable, effect or phenotype and the second such as the second is in response to the first, the second is a consequence of the first, or the second is caused by the first.

In some embodiments, when the ABP of the invention acts as an inhibitor or antagonist of expression, function, activity and/or stability of said OR10H1 or variant, it increases type-I cytokine secretion of immune cells (e.g., activated CTLs), such as one or more cytokines independently selected from the list consisting of: IFN-gamma, IL-2 and TNF-alpha. In certain embodiments, this increase in type-I cytokine secretion is accompanied by a decrease in type-II cytokine secretion (e.g. IL-4, IL-9, IL-13) of the immune cells (e.g. activated CTLs). Methods to determine such patterns of cytokine secretion are known to the person of ordinary skill, or can be found disclosed elsewhere herein. In particular, the assay(s) described in Example 2 may be used to determine the patterns of cytokine secretion from CTLs.

Analogous to that described above, the effects described herein as “reduced” or “decreased” upon or associated with the ABP that modulates the expression, function, activity and/or stability of said OR10H1 or variant, can be considered relative to the newly described finding of OR10H1-mediated resistance to one or more immune responses. That is, such reduction or decrease may be one relative to an ABP-associated “release” of such OR10H1-mediated reduction of an immune response in a given subject or with given cells. However, analogously such a “reduction” or “decrease” may be described as a reversion to a level typically present in a healthy subject or with normal cells. By way of one limiting specific example, while an ABP of the present invention may be described as “decreasing” CREB phosphorylation in immune cells, it may also be described as “preventing an increase” in such CREB phosphorylation.

In certain of such embodiments of the invention, the ABP of the invention that binds to one or more epitope(s) displayed by one or more extracellular domain(s) of OR10H1, or a paralogue, orthologue or other variant thereof will bind to said OR10H1 or variant with an EC50 of less than about 100 μM, 10 μM, 1 μM, 100 nM or 10 nM, preferably of less than about 1 μM. In another preferred such embodiment, the ABP of the invention will bind to said OR10H1 or variant with an EC50 of less than 1 nM. In a more such preferred embodiment, the ABP of the invention will bind to said OR10H1 or variant with an EC50 of less than 500 pM. In a most preferred such embodiment, the ABP of the invention will bind to said OR10H1 or variant with an EC50 of less than 100 pM. In other certain embodiments, the binding of an ABP of the invention, such as an antibody of the invention, to a human cell line expressing said OR10H1 or variant may occur at an EC50 of less than about 10 μg/mL, 5 μg/mL, 2 μg/mL, 1 μg/mL, 0.5 μg/mL or 0.2 μg/mL, preferably with an EC50 or less than 2 μg/mL. Binding of an ABP of the invention, such as an antibody of the invention, to a Cynomolgus cell line expressing an orthologue of said OR10H1 or variant may, in some other embodiments, occur at an EC50 of less than about 10 μg/mL, 5 μg/mL, 2 μg/mL, 1 μg/mL, 0.5 μg/mL or 0.2 μg/mL, preferably with an EC50 or less than 2 μg/mL.

In certain of such embodiments, the effector group comprised in the ABP may be a cytotoxic agent that induces cytotoxicity (e.g., apoptosis or mitotic arrest) such as one selected from the group consisting of: doxorubicin, doxorubicin derivatives, mertansine, ricin A toxin, alkylating agents (e.g., CC-1065 analogs), anthracyclines, calicheamicins, pyrrolobenzodiazepine, tubulysin analogs, duocarmycin analogs, streptonigtin, geldanamycin, centanamycin, cam ptothecin analogs (e.g., SN38), myatansinoids (e.g., maytansine, maytansinol, C-3 ester of maytansinol) and auristatin E. Alternatively the effector group comprised in the ABP may be to a radioisotope such as one selected from the group consisting of: Phosphorus-32, Strontium-89, Yttrium-90, Iodine-131, Samarium-153, Erbium-169, Ytterbium-175 and Rhenium-188.

In certain other of such embodiments, the ABP of the invention is labelled, for example it comprises a (detectable) labelling group, such as an isotopic label, a magnetic label, an optical dye, an enzymatic group, a biotinylated group or a predetermined polypeptide epitope recognized by a secondary reporter.

Nucleic Acids

In another aspect, herein provided is a nucleic acid that encodes an ABP of the invention or a component of said ABP, such as one as described above. For example, the component encoded by a nucleic acid of the invention may be all or part of one chain of an antibody of the invention; or the component may be a scFV of said ABP. The component encoded by such a nucleic acid may be all or part of both chains of an antibody of the invention. The nucleic acids of the invention may also encode a fragment, derivative, mutant, or variant of an ABP of the invention, and/or represent components that are polynucleotides sufficient for use as hybridization probes, polymerase chain reaction (PCR) primers or sequencing primers for identifying, analyzing, mutating or amplifying a polynucleotide encoding a polypeptide, anti-sense or inhibitory nucleic acids (such as RNAi/siRNA molecules) for inhibiting expression of a polynucleotide, and complementary sequences of the foregoing.

The nucleic acid according to the invention may be a DNA or RNA of genomic, mRNA, cDNA, or synthetic origin or some combination thereof, optionally linked to a polynucleotide to which it is not linked in nature. In some embodiments, such nucleic acid may comprise one or more (such as 2, 3, 4, 5, 6, 7, 8, 9, 10 or more than 20, in particular between 1 and about 5, or preferably all instances of a particular nucleotide in the sequence) unnatural (e.g. synthetic) nucleotides; and/or such nucleic acid may comprise (e.g. is conjugated to) another chemical moiety, such as a labelling group or an effector group; for example a labelling group or an effector group as described elsewhere herein.

In one embodiment the nucleic acid of the invention may be isolated or substantially pure. In another embodiment, the nucleic acid of the invention may be recombinant, synthetic and/or modified, or in any other way non-natural. For example, a nucleic acid of the invention may contain at least one nucleic acid substitution (or deletion) modification (such as 2, 3, 4, 5, 6, 7, 8, 9, 10 or more than 10 such modifications, in particular between 1 and about 5 such modifications, preferably 2 or 3 such modifications) relative to a product of nature, such as a human nucleic acid.

The nucleic acids can be any suitable length, such as about 10, 15, 20, 25, 30, 35, 40, 45, 50, 75, 100, 125, 150, 175, 200, 250, 300, 350, 400, 450, 500, 750, 1,000, 1,500, 3,000, 5,000 or more nucleotides in length. For example: siRNA nucleic acids may, preferably, be between about 15 to about 25 base pairs in length (preferably between about 19 and about 21 base pairs in length); shRNA nucleic acids may, preferably, comprise a 20-30 base pair stem, a loop of at least 4 nucleotides, and a dinucleotide overhang at the 3′ end; microRNA may, preferably, be about 22 base pairs in length; an mRNA or DNA sequence encoding an ABP or a component thereof (such as a heavy or light chain or an IgG antibody) of the invention may, preferably, be between about 500 and 1,500 nucleotides. More preferably, a nucleic acid encoding a mammalian light chain of an antibody may be between about 630 and about 650 nucleotides, and one encoding a mammalian heavy chain of an antibody may be between about 1,300 and about 1,650 nucleotides. A nucleic acid can comprise one or more additional sequences, for example, regulatory sequences, and/or be part of a larger nucleic acid. The nucleic acids can be single-stranded or double-stranded and can comprise RNA and/or DNA nucleotides, and artificial variants thereof (e.g., peptide nucleic acids).

Nucleic acids encoding antibody polypeptides (e.g., heavy or light chain, variable domain only, or full length) may be isolated from B-cells of mice, rats or rabbits that have been immunized with an OR10H1 antigen or fragment thereof, such as one or more EC domains (or a polynucleotide encoding and capable of expressing an OR10H1 antigen or fragment thereof). The nucleic acid may be isolated by conventional procedures such as PCR.

Changes can be introduced by mutation into the sequence of a nucleic acid of the invention. Such changes, depending on their nature and location in a codon, can lead to changes in the amino acid sequence of a polypeptide (e.g., an antigen binding protein) that it encodes. Mutations can be introduced using any technique known in the art.

In one embodiment, one or more particular amino acid residues may be changed using, for example, a site-directed mutagenesis protocol. In another embodiment, one or more randomly selected residues may be changed using, for example, a random mutagenesis protocol. However it is made, a mutant polypeptide can be expressed and screened for a desired property. Mutations can be introduced into a nucleic acid without significantly altering the biological activity of a polypeptide that it encodes. For example, one can make nucleotide substitutions leading to amino acid substitutions at non-essential amino acid residues.

Other changes that may be made (e.g. by mutation) to the sequence of a nucleic acid of the invention may not alter the amino acid sequence of the encoded polypeptide, but may lead to changes to its stability and/or effectiveness of expression of the encoded polypeptide. For example, by codon optimisation, the expression of a given polypeptide sequence may be improved by utilising the more common codons for a given amino acid that are found for the species in which the nucleotide is to be expressed. Methods of codon optimisation, and alternative methods (such as optimisation of CpG and G/C content), are described in, for example, Hass et al, 1996 (Current Biology 6:315); WO1996/09378; WO2006/015789 and WO 2002/098443).

In another aspect, herein provided is a nucleic acid construct (NAC) comprising a nucleic acid as described above and one or more additional features permitting the expression of the encoded ABP or component of said ABP in a cell (such as in a host cell). Examples of NACs of the invention include, but are not limited to, plasmid vectors, viral vectors, mRNA, non-episomal mammalian vectors and expression vectors, for example, recombinant expression vectors. The nucleic acid constructs of the invention can comprise a nucleic acid of the invention in a form suitable for expression of the nucleic acid in a cell, such as a host cell, (see below). The nucleic acid constructs of the invention will be, typically, recombinant nucleic acids, and/or may be isolated and/or substantially pure. Recombinant nucleic acids will, typically, be non-natural; particularly if they comprise portions that are derived from different species and/or synthetic, in-vitro or mutagenic methods.

In some embodiments, an NAC of the invention comprises one or more constructs either of which includes a nucleic acid encoding either a heavy or a light antibody chain. In some embodiments, the NAC of the invention comprises two constructs, one of which includes a nucleic acid encoding the heavy antibody chain, the other of which includes a nucleic acid encoding the light antibody chain, such that expression from both constructs can generate a complete antibody molecule. In some embodiments, the NAC of the invention comprises a construct which includes nucleic acids encoding both heavy and light antibody chains, such that a complete antibody molecule can be expressed from one construct. In other embodiments, an NAC of the invention can comprise a single construct that encodes a single chain which is sufficient to form an ABP of the invention; for example, if the encoded ABP is a scFv or a single-domain antibody (such as a camelid antibody).

In some embodiments, the NAC of the invention includes sequences encoding all or part of a constant region, enabling an entire, or a part of, a heavy and/or light chain to be expressed.

An NAC according to the invention may comprise (or consist of) a mRNA molecule which includes an open reading frame encoding an ABP of the invention, and for example together with upstream and downstream elements (such as 5′ and/or 3′ UTRs and/or poly-A stretch) that enables expression of the ABP, and preferably enhancing stability of the mRNA and/or expression of the ABP. The use of mRNA as NACs to introduce into and express polynucleotides in cells is described, for example, in Zangi et al in Nat. Biotechnol. vol. 31, 898-907 (2013), Sahin et al (2014) Nature Reviews Drug Discovery 13:759 and by Thess et al in Mol. Ther. vol. 23 no. 9, 1456-1464 (2015). Particular UTRs that may be comprised in an mRNA NAC of the invention include: 5′UTR of a TOP gene (WO2013/143699), and/or a histone stem-loop (WO 2013/120629). An mRNA NAC of the invention may further comprise one or more chemical modifications (EP 1 685 844); including a 5′-cap, such as m7G(5′)ppp, (5′(A,G(5′)ppp(5′)A or G(5′)ppp(5′)G and/or at least one nucleotide that is an analogue of naturally occurring nucleotides, such as phosphorothioates, phosphoroamidates, peptide nucleotides, methylphosphonates, 7-deaza-guanosine, 5-methylcytosine or inosine.

NACs, such as DNA-, retroviral- and mRNA-based NACs of the invention may be used in genetic therapeutic methods in order to treat or prevent diseases of the immune system (see Methods of Treatment below), whereby an NAC that comprises an expressible sequence encoding an ABP of the invention is administered to the cell or organism (e.g. by transfection). In particular, the use of mRNA therapeutics for the expression of antibodies is known from WO2008/083949.

In another aspect, the invention relates to a cell, such as a host cell, comprising one or more NAC(s) of the invention. Preferably, such cell is capable of expressing the ABP (or component therein) encoded by said NAC(s). For example, if an ABP of the invention comprises two separate polypeptide chains (e.g. a heavy and light chain of an IgG), then the cell of the invention may comprise a first NAC that encodes (and can express) the heavy chain of such ABP as well as a second NAC that encodes (and can express) the light chain of such ABP; alternatively the cell may comprise a single NAC that encodes both chains of such ABP. In these ways, such a cell of the invention would be capable of expressing a functional (e.g. binding and/or inhibitory) ABP of the invention. A (host) cell of invention may be one of the mammalian, prokaryotic or eukaryotic host cells as described elsewhere herein.

In certain embodiments of such aspect, the (host) cell is a human cell; in particular it may be a human cell that has been sampled from a specific individual. In such embodiments, such human cell can be propagated and/or manipulated in-vitro so as to introduce a NAC of the present invention. The utility of a manipulated human cell from a specific individual can be to produce an ABP of the invention, including to reintroduce a population of such manipulated human cells into a human subject, such as for use in therapy. In certain of such uses, the manipulated human cell may be introduced into the same human individual from which it was first sampled; for example as an autologous human cell.

The human cell that is subject to such manipulation can be of any germ cell or somatic cell type in the body. For example, the donor cell can be a germ cell or a somatic cell selected from the group consisting of fibroblasts, B cells, T cells, dendritic cells, keratinocytes, adipose cells, epithelial cells, epidermal cells, chondrocytes, cumulus cells, neural cells, glial cells, astrocytes, cardiac cells, oesophageal cells, muscle cells, melanocytes, hematopoietic cells, macrophages, monocytes, and mononuclear cells. The donor cell can be obtained from any organ or tissue in the body; for example, it can be a cell from an organ selected from the group consisting of liver, stomach, intestines, lung, pancreas, cornea, skin, gallbladder, ovary, testes, kidneys, heart, bladder, and urethra.

Pharmaceutical Compositions

To be used in therapy, the ABPs or NACs (or the cells, such as host cells) of the invention may be formulated into a pharmaceutical composition appropriate to facilitate administration to animals or humans. In those embodiments using (host) cells, typically the same species (or individual) of cells may be used as the species (or individual) into which the cells are administered. Accordingly, in another aspect, herein provided is a pharmaceutical composition comprising an ABP of the invention, and/or at least one NAC of the invention, and/or a (host) cell of the invention, and a pharmaceutically acceptable excipient or carrier. In a preferred embodiment, the pharmaceutical composition comprises an ABP of the invention.

By way of example, the pharmaceutical composition of the invention may comprise between 0.1% and 100% (w/w) active ingredient (for example, an ABP or NAC of the invention), such as about 0.5%, 1%, 2%, 3%, 4%, 5%, 6%, 8% 10%, 15%, 20%, 25%, 30%, 40%, 50%, 60%, 70%, 80%, 90%, 95%, 96%, 97%, 98% or 99%, preferably between about 1% and about 20%, between about 10% and 50% or between about 40% and 90%.

Pharmaceutical compositions containing ABPs (e.g., antibodies or antibody fragments) and/or NACs and/or (host) cells of the invention can be presented in any suitable dosage form (such as unit dosage form) and can be prepared by any suitable method.

Remington's The Science and Practice of Pharmacy, 1st Edition, A . R . Gennaro (Lippincott, Williams & Wilkins, Baltimore, Md., 2006; incorporated herein by reference) discloses various excipients and carriers used in formulating pharmaceutical compositions and known techniques for the preparation thereof. Except insofar as any conventional excipient or carrier medium is incompatible with a substance or its derivatives, such as by producing any undesirable biological effect or otherwise interacting in a deleterious manner with any other component(s) of the pharmaceutical composition, its use is contemplated to be within the scope of this invention.

Typical formulations of an ABP and/or NAC molecule can be prepared by mixing the ABP and/or NAC molecule with physiologically acceptable carriers, excipients or stabilizers, in the form of lyophilized or otherwise dried formulations or aqueous solutions or aqueous or non-aqueous suspensions. Carriers, excipients, modifiers or stabilizers are nontoxic at the dosages and concentrations employed. They include buffer systems such as phosphate, citrate, acetate and other anorganic or organic acids and their salts; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; metal complexes (e.g. Zn-protein complexes); and/or ionic or non-ionic surfactants such as TWEEN™ (polysorbates), PLURONICS™ or fatty acid esters, fatty acid ethers or sugar esters. Also organic solvents can be contained in the antibody formulation such as ethanol or isopropanol. The excipients may also have a release-modifying or absorption-modifying function.

The ABP and/or NAC molecules may also be dried (freeze-dried, spray-dried, spray-freeze-dried, dried by near or supercritical gases, vacuum dried, air-dried), precipitated or crystallized or entrapped in microcapsules that are prepared, for example, by coacervation techniques or by interfacial polymerization using, for example, hydroxymethylcellulose or gelatin and poly-(methylmethacylate), respectively, in colloidal drug delivery systems (for example, liposomes, albumin microspheres, microemulsions, (lipid)nano-particles and nanocapsules), in macroemulsions or precipitated or immobilized onto carriers or surfaces, for example by pcmc (protein coated microcrystals) technology. Such techniques are disclosed in Remington: The Science and Practice of Pharmacy, 21st edition, Hendrickson R. Ed.

Naturally, the formulations to be used for in vivo administration must be sterile; sterilization may be accomplished be conventional techniques, e.g. by filtration through sterile filtration membranes. It may be useful to increase the concentration of the antibody to come to a so-called high concentration liquid formulation (HCLF); various ways to generate such HCLFs have been described.

The ABP and/or NAC molecule may also be contained in a sustained-release preparation. Such preparations include solid, semi-solid or liquid matrices of hydrophobic or hydrophilic polymers, and may be in the form of shaped articles, e.g. films, sticks or microcapsules and may be applied via an application device. Examples of sustained-release matrices include polyesters, hydrogels (for example, poly(2-hydroxyethyl-methacrylate) or sucrose acetate butyrate), or poly(vinylalcohol)), polylactides (U.S. Pat. No. 3,773,919), copolymers of L-glutamic acid and [gamma] ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated ABPs (e.g. antibodies) remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thiol-disulfide interchange, stabilization may be achieved by modifying sulfhydryl residues, lyophilization (e.g. as described in WO 89/011297) from acidic solutions, controlling moisture content, using appropriate additives, and developing specific polymer matrix compositions. Formulations that may also be used for the antibody molecule of the invention are described in U.S. Pat. Nos. 7,060,268 and 6,991,790.

The ABP and/or NAC molecule can be incorporated also in other application forms, such as dispersions, suspensions or liposomes, tablets, capsules, powders, sprays, transdermal or intradermal patches or creams with or without permeation-enhancing devices, wafers, nasal, buccal or pulmonary formulations, or may be produced by implanted cells or—after gene therapy—by the individual's own cells.

In particular embodiments, the NACs of the invention may be combined with (or comprised in) agents that enhance the stability of such NAC and/or enhance the efficiency of their transfection into cells of the subject administered with the pharmaceutical composition. Such agents include cationic transfection agents, peptides/polypeptides (especially cationic polypeptides) and lipid nanoparticles (for example, those comprising a cationic lipid, a non-cationic lipid and a conjugated lipid that inhibits the aggregation of particles).

An ABP and/or NAC molecule may also be derivatized with a chemical group such as polyethylene glycol (PEG), a methyl or ethyl group, or a carbohydrate group. These groups may be useful to improve the biological characteristics of the ABP and/or NAC, e.g. to increase serum half-life or to increase tissue binding.

Cells, such as host cell, of the invention can be included in pharmaceutical formulations suitable for administration into the bloodstream or for administration directly into tissues or organs. A suitable format is determined by the skilled person (such as a medical practitioner) for each patient, tissue, and organ, according to standard procedures. Suitable pharmaceutically acceptable carriers and their formulation are known in the art (see, e.g. Remington's Pharmaceutical Sciences 16th edition, Osol, A. Ed., 1980). Cells of the invention, when formed in a pharmaceutical composition, are preferably formulated in solution at a pH from about 6.5 to about 8.5. Excipients to bring the solution to isotonicity can also be added, for example, 4.5% mannitol or 0.9% sodium chloride, pH buffered with art-known buffer solutions, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes. In one embodiment, a media formulation is tailored to preserve the cells while maintaining cell health and identity. For example, a premixture including an aqueous solution of anticoagulant (ACD-A), an equal amount of dextrose (50%), and phosphate buffered saline (PBS), or the like is pre-mixed and aliquoted in a volume to typically match or approximate the cellular matrix or environment from which the cell was extracted from the tissue or organ.

In one embodiment is provided a pharmaceutical composition further comprising a labelling group or an effector group. In yet another embodiment is provided a pharmaceutical composition wherein the labelling group is selected from the group consisting of isotopic labels, magnetic labels, redox active moieties, optical dyes, biotinylated groups and predetermined polypeptide epitopes recognized by a secondary reporter. In yet another embodiment is provided a pharmaceutical composition wherein the effector group is selected from the group consisting of a radioisotope, radionuclide, a toxin, a therapeutic group and a chemotherapeutic group.

In some embodiments, the pharmaceutical composition comprising an ABP is in unit dose form of between 10 and 1000 mg ABP. In some embodiments, the pharmaceutical composition comprising an ABP is in unit dose form of between 10 and 200 mg ABP. In some embodiments, the pharmaceutical composition comprising an ABP is in unit dose form of between 200 and 400 mg ABP. In some embodiments, the pharmaceutical composition comprising an ABP is in unit dose form of between 400 and 600 mg ABP. In some embodiments, the pharmaceutical composition comprising an ABP is in unit dose form of between 600 and 800 mg ABP. In some embodiments, the pharmaceutical composition comprising an ABP is in unit dose form of between 800 and 1000 mg ABP.

In some embodiments, the pharmaceutical composition comprising an NAC is in unit dose form of between about 5 μg and about 5000 μg NAC, such as about 10 μg and about 1000 μg NAC. In some embodiments, the pharmaceutical composition comprising an NAC is in unit dose form of between about 10 μg and about 200 μg NAC. In some embodiments, the pharmaceutical composition comprising an NAC is in unit dose form of between about 200 μg and about 400 μg NAC. In some embodiments, the pharmaceutical composition comprising an NAC is in unit dose form of between about 400 μg and about 600 μg NAC. In some embodiments, the pharmaceutical composition comprising an NAC is in unit dose form of between about 600 μg and about 800 μg NAC. In some embodiments, the pharmaceutical composition comprising an NAC is in unit dose form of between about 800 μg and about 1000 μg NAC.

In some embodiments, the pharmaceutical composition comprising (host) cells is in unit dose form of between about 1×10\hat{ }3 and about 1×10\hat{ }9 cells (i.e. of the (host) cells). In some embodiments, the pharmaceutical composition comprising (host) cells is in unit dose form of between about 1×10\hat{ }4 and about 1×10\hat{ }8 cells. In some embodiments, the pharmaceutical composition comprising (host) cells is in unit dose form of between about 1×10\hat{ }5 and about 1×10\hat{ }7 cells. In some embodiments, the pharmaceutical composition comprising (host) cells is in unit dose form of about 1×10\hat{ }6, about 2×10\hat{ }6, about 5×10\hat{ }6 or about 8×10\hat{ }6 cells.

Exemplary unit dosage forms for pharmaceutical compositions comprising ABPs and/or NACs are tablets, capsules (e.g. as powder, granules, microtablets or micropellets), suspensions or as single-use pre-loaded syringes. In certain embodiments, kits are provided for producing a single-dose administration unit. The kit can contain both a first container having a dried protein and a second container having an aqueous formulation. Alternatively, the kit can contain single and multi-chambered pre-loaded syringes.

Modulating Uses, Medical Uses and Methods of Treatment

The ABPs, NAC, (host) cells and the pharmaceutical compositions of the invention can be used in various ways to modulate the expression, function, activity and/or stability of a human OR10H1 or paralogue, orthologue or other variant thereof, including their use in therapy or for prophylaxis.

Accordingly, in a further aspect, herein provided is a method of modulating the expression, function, activity and/or stability of a human OR10H1 or paralogue, orthologue or other variant thereof comprising contacting a cell that expresses said OR10H1 or variant with an ABP of the invention or an NAC encoding said ABP. When such ABP is a modulator of the expression, function, activity and/or stability of said OR10H1 or variant, thereby the expression, function, activity and/or stability of said OR10H1 or variant is modulated. Such method may be practiced on cells that are present ex-vivo, that is where said cells are contained in receptacles or containers, such as those used in research facilities. Accordingly, in such embodiments such method of the invention can be described as an in-vitro method of modulating the expression, function, activity and/or stability of a human OR10H1 or paralogue, orthologue or other variant thereof. However, in alternative embodiments, the method may be practiced using cells within the body, for example an in-vivo method of modulating the expression, function, activity and/or stability of a human OR10H1 or paralogue, orthologue or other variant thereof. In particular of such embodiments, such an in-vitro (or in-vivo) method comprises the inhibition of the function and/or activity of said OR10H1 or variant, when the ABP is an inhibitor of and/or antagonist of such function and/or activity. In some embodiments of such method, it further comprises the step of contacting the cell with an immune cell, such as a CTL or TIL. Preferably, the ABP is an antibody, or an antibody fragment, and is an inhibitor of the function and/or activity of said OR10H1 or variant.

In yet a further aspect, herein provided is a method of enhancing a cell-mediated immune response to a mammalian cell, such as a human cell, that expresses a human OR10H1 or paralogue, orthologue or other variant thereof, comprising contacting said cell with an ABP of the invention, or an NAC encoding said ABP, in the presence of an immune cell (in particular a CTL or TIL), such as a T-cell, wherein said ABP is an inhibitor of the expression, function, activity and/or stability of said OR10H1 or variant, thereby enhancing a cell-mediated immune response. Preferably, the ABP is an antibody, or an antibody fragment, and is an inhibitor of the function and/or activity of said OR10H1 or variant.

In preferred embodiments of the therapeutic aspects, the ABP (such as one that is an inhibitor or antagonist of expression, function, activity and/or stability of said OR10H1, paralogue, orthologue or other variant thereof), or an NAC encoding said ABP, in: (i) modulating the expression, function, activity and/or stability of a human OR10H1 or paralogue, orthologue or other variant thereof; and/or (ii) enhancing a cell-mediated immune response to a mammalian cell, decreases or reduces the resistance of cells (such as tumour cells) that express OR10H1, or the variant of OR10H1, to an immune response. In other certain preferred embodiments of the invention, the ABP (such as one that is an inhibitor or antagonist of expression, function, activity and/or stability of said OR10H1, paralogue, orthologue or other variant thereof), or an NAC encoding said ABP, enhances or increases the sensitivity of cells (such as tumour cells) that express OR10H1, or the variant of OR10H1, to an immune response.

The immune response, is, in particular of such embodiments, a cell-mediated immune response such as one mediated by T-cells including cytotoxic T-cells and/or TILs; and/or the immune response is the lysis and/or killing of the cells that express OR10H1 (or a paralogue, orthologue or other variant of OR10H1) that is mediated by cytotoxic T-cells and/or TILs. In other particular of such embodiments, the immune response is a cytotoxic immune response against cells (such as tumour cells) that express OR10H1 (or a paralogue, orthologue or other variant of OR10H1), in particular a cell-mediated cytotoxic immune response such as one mediated by T-cells including cytotoxic T-cells and/or TILs.

Specifically, in certain preferred embodiments of such therapeutic aspects the ABP (such as one that is an inhibitor or antagonist of expression, function, activity and/or stability of said OR10H1, paralogue, orthologue or other variant thereof), or an NAC encoding said ABP, enhances or increases killing and/or lysis of cells expressing OR10H1, or the variant of OR10H1, (such as tumour cells); preferably killing and/or lysis being mediated by cytotoxic T-cells and/or TILs, and/or mediated by an enhancement of or increase in the sensitivity of the cells expressing OR10H1 (or the variant of OR10H1) to a (cytotoxic) immune response, such an immune response described above, and/or mediated by a decrease in or reduction of the resistance of the cells expressing OR10H1 (or the variant of OR10H1) to a (cytotoxic) immune response, such an immune response described above.

The cells that express OR10H1 (or a paralogue, orthologue or other variant of OR10H1) are, in certain of such preferred embodiments, cancer cells or are cells that originated from a tumor cell. Exemplary cancer or tumor cells can be those as described or exemplified elsewhere herein.

In other certain preferred embodiments of such therapeutic aspects, the ABP (such as one that is an inhibitor or antagonist of expression, function, activity and/or stability of said OR10H1, paralogue, orthologue or other variant thereof), or an NAC encoding said ABP, increases T-cell activity and/or survival, which in certain embodiments, may lead to an enhancement of a (cytotoxic) immune response mediated by such T-cells.

In another aspect, herein provided is the ABP, the NAC, the (host) cells, or the pharmaceutical composition as described above, for use in medicine, such as in the treatment or the prevention of a disease, disorder or condition in a mammalian subject.

In an alternative aspect, herein provided is a method of treating or preventing a disease, disorder or condition in a mammalian subject in need thereof, comprising administering to said subject at least once an effective amount of the ABP, the NAC, the (host) cells, or the pharmaceutical composition as described above.

In other aspects described elsewhere herein, are provided methods to detect and/or diagnose a disease, disorder or condition in a mammalian subject.

In some particular embodiments, the disease, disorder or condition of the subject is a proliferative disorder (or a condition associated with such disorder).

A “proliferative disorder” refers to a disorder characterised by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “proliferative disorder” is neoplasm or tumour, which is an abnormal growth of tissue or cells. Cancer is art understood, and includes any of various malignant neoplasms characterised by the proliferation of cells that have the capability to invade surrounding tissue and/or metastasise to new colonisation sites. Proliferative disorders include cancer, atherosclerosis, rheumatoid arthritis, idiopathic pulmonary fibrosis and cirrhosis of the liver. Non-cancerous proliferative disorders also include hyperproliferation of cells in the skin such as psoriasis and its varied clinical forms, Reiter's syndrome, pityriasis rubra pilaris, and hyperproliferative variants of disorders of keratinization (e.g., actinic keratosis, senile keratosis), scleroderma, and the like.

In more particular embodiments, the proliferative disorder is a cancer or tumour, in particular a solid tumour (or a condition associated with such cancer or tumour). Such proliferative disorders include, but are not limited to, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chronic myelomonocytic leukemia, acute lymphoblastic leukemia, acute lymphocytic leukemia, acute myelogenous leukemia, acute myeloblasts leukemia, chronic myeloblastic leukemia, Hodgkin's disease, non-Hodgkin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, marginal zone lymphoma, Burkitt Lymphoma, mycosis fungoides, seary syndrome, cutaneous T-cell lymphoma, peripheral T cell lymphoma, chronic myeloproliferative disorders, myelofibrosis, myeloid metaplasia, systemic mastocytosis), and cental nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelfibrosis), soft tissue sarcoma, thyroid cancer, endometrial cancer, carcinoid cancer, or liver cancer.

In one preferred embodiment, the various aspects of the invention relate to, for example the ABPs of the invention used to detect/diagnose, prevent and/or treat, such proliferative disorders that include but are not limited to carcinoma (including breast cancer, prostate cancer, lung cancer, colorectal and/or colon cancer, hepatocellular carcinoma, melanoma), lymphoma (including non-Hodgkin's lymphoma and mycosis fungoides), leukemia, sarcoma, mesothelioma, brain cancer (including glioma), germinoma (including testicular cancer and ovarian cancer), choriocarcinoma, renal cancer, pancreatic cancer, thyroid cancer, head and neck cancer, endometrial cancer, cervical cancer, bladder cancer, or stomach cancer.

In a more preferred embodiment, the various aspects of the invention relate to, for example the ABPs of the invention used to detect/diagnose, prevent and/or treat, one or more proliferative disorder(s) selected from the list consisting of: melanoma, pancreatic cancer and colorectal cancer (or a condition associated with such cancer or tumour).

According to the medical uses and methods of treatment disclosed herein, the subject is a mammal, and may include mice, rats, rabbits, monkeys and humans. In a preferred embodiment, the mammalian subject is a human patient.

In the context of the invention, an effective amount of the ABP, the NAC, the (host) cells, or the pharmaceutical composition can be one that will elicit the biological, physiological, pharmacological, therapeutic or medical response of a cell, tissue, system, body, animal, individual, patient or human that is being sought by the researcher, scientist, pharmacologist, pharmacist, veterinarian, medical doctor, or other clinician, e.g., lessening of the effects/symptoms of a disorder, disease or condition, such as a proliferative disorder or disease, for example, a cancer or tumour, or killing or inhibiting growth of a proliferating cell, such as a tumour cell. The effective amount can be determined by standard procedures, including those described below.

In accordance with all aspects and embodiments of the medical uses and methods of treatment provided herein, the effective amount administered at least once to a subject in need of said ABP is between about 0.01 mg/kg and about 100 mg/kg per administration, such as between about 1 mg/kg and about 10 mg/kg per administration. In some embodiments, the effective amount administered at least once to said subject of said ABP is between about 0.01 mg/kg and about 0.1 mg/kg per administration, between about 0.1 mg/kg and about 1 mg/kg per administration, between about 1 mg/kg and about 5 mg/kg per administration, between about 5 mg/kg and about 10 mg/kg per administration, between about 10 mg/kg and about 50 mg/kg per administration, or between about 50 mg/kg and about 100 mg/kg per administration.

In accordance with all aspects of the medical uses and methods of treatment provided herein, the effective amount administered at least once to said subject of said NAC is between about 0.01 μg/kg and about 1000 μg/kg per administration. In some embodiments, the effective amount administered at least once to said subject of said NAC is between about 0.05 μg/kg and about 500 μg/kg per administration, between about 0.1 μg/kg and about 100 μg/kg per administration, between about 10 pg/kg and about 50 μg/kg per administration, between about 50 μg/kg and about 100 μg/kg per administration, or between about 100 μg/kg and about 250 μg/kg per administration, or between about 250 μg/kg and about 500 μg/kg per administration.

In accordance with all aspects of the medical uses and methods of treatment provided herein, the effective amount administered at least once to said subject of said (host) cells is between about 5 cells/kg and about 1×10\hat{ }8 cells/kg per administration. In some embodiments, the effective amount administered at least once to said subject of said (host) cells is between 10 cells/kg and about 1×10\hat{ }7 cells/kg, between about 50 cells/kg and about 5×10\hat{ }6 cells/kg per administration, between about 100 cells/kg and about 1×10\hat{ }6 cells/kg per administration, between about 10×10\hat{ }3 cells/kg and about 1×10\hat{ }5 cells/kg per administration, or between about 10×10\hat{ }5 cells/kg and about 1×10\hat{ }7 per administration.

For the prevention or treatment of disease, the appropriate dosage of ABP (e.g. antibody) and/or NAC and/or (host) cells (or a pharmaceutical composition comprised thereof) will depend on the type of disease to be treated, the severity and course of the disease, whether the ABP and/or NAC and/or (host) cells and/or pharmaceutical composition is administered for preventive or therapeutic purposes, previous therapy, the patient's clinical history, age, size/weight and response to the ABP and/or NAC and/or (host) cells and/or pharmaceutical composition, and the discretion of the attending physician. The ABP and/or NAC and/or (host) cells and/or pharmaceutical composition is suitably administered to the patient at one time or over a series of treatments. If such ABP and/or NAC and/or (host) cells and/or pharmaceutical composition is administered over a series of treatments, the total number of administrations for a given course of treatment may consist of a total of about 2, 3, 4, 5, 6, 7, 8, 9, 10 or more than about 10 treatments. For example, a treatment may be given once every day (or 2, 3 or 4 times a day) for a week, a month or even several months. In certain embodiments, the course of treatment may continue indefinitely.

The amount administered will depend on variables such as the type and extent of disease or indication to be treated, the overall health, age, size/weight of the patient, the in vivo potency of the ABP, NAC and/or (host) cell preparation, the pharmaceutical composition, and the route of administration. The initial dosage can be increased beyond the upper level in order to rapidly achieve the desired blood-level or tissue level. Alternatively, the initial dosage can be smaller than the optimum, and the daily dosage may be progressively increased during the course of treatment. Human dosage can be optimized, e.g., in a conventional Phase I dose escalation study designed to run from relatively low initial doses, for example from about 0.01 mg/kg to about 20 mg/kg of antibody. Dosing frequency can vary, depending on factors such as route of administration, dosage amount and the disease being treated. Exemplary dosing frequencies are once per day, once per week and once every two weeks. Formulation of an ABP of the invention (such as monoclonal antibody-based drugs) or of a NAC of the present invention or of a (host) cell of the invention, is within the ordinary skill in the art. In some embodiments of the invention such an ABP or NAC is lyophilized and reconstituted in buffered saline at the time of administration. The ABP and/or NAC and/or (host) cells and/or pharmaceutical composition of the present invention may further result in a reduced relapsing of the disease to be treated or reduce the incidence of drug resistance or increase the time until drug resistance is developing; and in the case of cancer may result in an increase in the period of progression-free survival and/or overall survival.

An ABP (e.g. an antibody) of the invention which is an inhibitor/antagonist of OR10H1 and which enhances an immune response, eg, of an activated CTL in a subject is expected to be useful in the prevention and treatment of various diseases, disorders or conditions requiring an up-regulated immune response, e.g. a proliferative disorder (such as a tumour or cancer), or an infection, or an immune disorder (such as an immune deficiency).

Accordingly, in some embodiments of the medical uses and methods of treatment provided herein, the disease, disorder or condition to be treated or prevented by the ABP, NAC, (host) cells or pharmaceutical composition is a tumour or cancer.

In a preferred embodiment of the medical uses and methods of treatment provided herein, the disease, disorder or condition to be treated or prevented is cancer.

ABPs, NACs, (host) cells and pharmaceutical compositions provided herein can be used to treat various forms of cancer, including but not limited to the following: AIDS-related cancer, bone related cancer, brain related cancer, digestive/gastrointestinal related cancer, endocrine related cancer, eye related cancer, genitourinary related cancer, germ cell related cancer, gynaecologic related cancer, head and neck related cancer, hematologic/blood related cancer such as leukaemia, lung related cancer such as non-small cell lung cancer and small cell lung cancer, musculoskeletal related cancer, neurologic related cancer, respiratory/thoracic related cancer, and skin related cancer. These disorders have been well characterized in man, but also exist with a similar etiology in other mammals, and can be treated by ABPs, NACs and pharmaceutical compositions of the present invention.

An infection in the context of the present invention is any infection known in the art, such as a bacterial or a viral infection, preferably a viral infection, in particular a viral infection wherein the immune system malfunctions, i.e., overreacts (e.g., some forms of influenza) or reacts too weakly (human immune deficiency virus (HIV) infections, herpes, warts).

In some embodiments of the medical uses and methods of treatment provided herein, the disease, disorder or condition to be treated or prevented is a cancer expressing said OR10H1 or variant; for example an OR10H1-positive cancer. In such embodiments, an ABP of the invention which is an inhibitor or antagonist of expression, function, activity and/or stability of said OR10H1 or variant is expected to be useful in said medical uses and methods of treatment.

In preferred embodiments of the medical uses and methods of treatment provided herein, the disease, disorder or condition to be treated or prevented is selected from the list consisting of: melanoma, pancreatic cancer and colorectal cancer.

In some embodiments of the medical uses and methods of treatment provided herein, the ABPs, NACs, (host) cells and/or pharmaceutical compositions of the present invention inhibit cancer cell proliferation by at least 30%, 40%, 50%, 60%, 70%, 80%, 90%, 95%, 98%, 99%, or 100%, in particular between about 30% and 60%, preferably at least about 40% or 50%.

Depending on the disorder to be treated, the ABP and/or NAC and/or (host) cells and/or pharmaceutical composition of the invention may be used on its own or in combination with one or more additional therapeutic agents, in particular selected from DNA damaging or tubulin binding agents or therapeutically active compounds that inhibit angiogenesis, signal transduction pathways or mitotic checkpoints in cancer cells. The additional therapeutic agent may be administered simultaneously with the ABPs, NACs or pharmaceutical compositions of the invention, optionally as a component of the same pharmaceutical preparation, or before or after administration of the ABPs, NACs or pharmaceutical compositions of the invention. Moreover, an ABP of the invention may be used on its own or in the form of a conjugate, i.e., an ABP molecule that is chemically coupled to a cytotoxic agent that induces cytotoxicity (e.g., apoptosis or mitotic arrest) such as doxorubicin, doxorubicin derivatives, mertansine, ricin A toxin, alkylating agents (e.g., CC-1065 analogs), anthracyclines, calicheamicins, pyrrolobenzodiazepine, tubulysin analogs, duocarmycin analogs, streptonigtin, geldanamycin, centanamycin, cam ptothecin analogs (e.g., SN38), myatansinoids (e.g., maytansine, maytansinol, C-3 ester of maytansinol) and auristatin E. Alternatively the ABP may also be coupled to a radioisotope such as Phosphorus-32, Strontium-89, Yttrium-90, Iodine-131, Samarium-153, Erbium-169, Ytterbium-175 and Rhenium-188.

The preferred mode of application of ABPs, NACs and pharmaceutical compositions according to the methods of the present invention is parenteral, by infusion or injection (intravenous, intramuscular, subcutaneous, intraperitoneal, intradermal), but other modes of application such as by inhalation, transdermal, intranasal, buccal, oral, or rectal may also be applicable. The route of administration of the ABP and/or NAC and/or (host) cells and/or pharmaceutical composition of the present invention will be that which is appropriate in the light of various factors, including but not limited to the pharmaceutical form, the disease, disorder or condition to be treated and/or suitable for the patient. Accordingly, all routes of administration are envisioned for the ABP and/or NAC and/or (host) cells and/or pharmaceutical composition of the present invention; including but not limited to systemic, oral, nasal, topical (including buccal and sublingual), rectal, vaginal and/or parenteral administration. For systemic administration, injection is preferred, including intramuscular, intravenous, intraperitoneal, and subcutaneous (i.m., i.v., i.p., and s.c. respectively) injection. Systemic administration can also be by transmucosal or transdermal routes and/or (other) means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories.

In some embodiments of the medical uses and methods of treatment provided herein, the disease, disorder or condition to be treated or prevented by the ABP, NAC (host) cells or pharmaceutical composition is characterised by (aberrant) expression and/or activity of a human OR10H1, or a paralogue, orthologue or other variant thereof. In other embodiments of the medical uses and methods of treatment provided herein, the disease, disorder or condition to be treated or prevented by the ABP, NAC, (host) cells or pharmaceutical composition is characterised by an aberrant (e.g. overactive) immune response. In other embodiments of the medical uses and methods of treatment provided herein, the disease, disorder or condition to be treated or prevented is characterized as a localised expression (e.g. an aberrant expression) of said OR10H1 or variant, or another localised abnormality, such as on tumour cells.

In some embodiments of the medical uses and methods of treatment provided herein, the disease, disorder or condition to be treated or prevented is associated with a pathological CTL response.

When a cellular CTL response occurs, this typically results in the death of the cell contacted with the CTL. The cell used in the context of the invention may be any cell that can be involved in a CTL response, i.e., preferably cells of mammalian origin, in particular cells of human origin. Most preferably, the cells are involved in abnormal growth cells such as, e.g., neoplastic cells, in particular cancer cells. Then tumour resistance may be mediated. A pathological CTL response is one that is too weak or that is too strong, i.e., one that differs from the healthy or physiological/immunological normal condition, and such differing CTL response can lead to a pathological disorder (e.g. a disease or condition), being any condition that differs from the healthy condition.

When a CTL response is too weak, pathologic cells may erroneously not be removed (killed), such as, e.g., neoplastic cells or infected cells. Then, exemplarily, neoplasia, in particular cancer, may occur or infections may be promoted.

In particular embodiments of the ABP, NAC, (host) cells, or pharmaceutical composition for the medical use, or the method of treatment, said ABP is an inhibitor or antagonist of expression, function, activity and/or stability of said OR10H1 or variant; preferably is an inhibitor or antagonist of the function and/or activity of said OR10H1 or variant.

In some embodiments of the medical uses and methods of treatment provided herein, a cell-mediated immune response (such as of an activated CTL) is enhanced in said subject, for example the ABP, NAC, (host) cells or pharmaceutical composition. Alternatively, or in addition, immune cell (such as T-cell, in particular CTL or TIL) activity and/or survival is increased in said subject, for example by the ABP, NAC, (host) cells or pharmaceutical composition.

In some embodiments of the medical uses and methods of treatment provided herein, type-I cytokine secretion by said immune cells (such as CTLs) is increased in said subject, for example by the ABP, NAC, (host) cells or pharmaceutical composition. A type-I cytokine that shows such increase can be one or more cytokines independently selected from the list consisting of: IFN-gamma, IL-2 and TNF-alpha.

In some embodiments of the medical uses and methods of treatment provided herein, the enhancement of a cell-mediated immune response such as that mediated by an immune cell (e.g., an activated CTL), and/or the increase in immune cell (such as T-cell in particular CTL or TIL) activity and/or survival by the ABP, NAC, (host) cells or pharmaceutical composition, is associated with: (x) reduced phosphorylation of CREB in such immune cell(s); and/or (y) activation of lymphocyte-specific protein tyrosine kinase (Lck), for example by reduced phosphorylation of the LcK Tyr505 domain in such immune cell(s).

In some of such embodiments, the increase in said immune cell activity and/or survival is associated with reduced phosphorylation of protein kinase A (PKA) in said immune cells.

In further of such embodiments the increase in said immune cell activity and/or survival is associated with a reduction in the level of cAMP in said immune cells. In certain of such embodiments, the reduction in the level of cAMP in said immune cells is associated with a reduction in the transport of cAMP into said immune cells from one or more other cells (such as a tumour cell expressing said OR10H1 or variant) via a gap-junction protein, for example connexin 32 (Cx32), expressed by said other cells.

Without being bound by theory, such association may mean the observed effect is correlated and/or related to, for example the effect is a surrogate marker for the biological response; or such association may have a causative relationship, such that the biological response is in response to, a consequence of or caused by the observed effect.

In a certain aspect, the invention also relates to a method of treating a disease, disorder or condition in a mammalian subject (such as a human patient), in particular one associated with or characterised by (aberrant) expression and/or activity of a human OR10H1, or a paralogue, orthologue or other variant thereof (such as an OR10H1-positive tumour and/or one that is resistant to a T-cell mediated immune response), the method comprising the step of administering to the subject a therapeutically effective amount of: (a) an inhibitor or antagonist of human OR10H1, or a paralogue, orthologue or other variant thereof; and (b) an inhibitor or antagonist of Cx32, CSK and/or PKA.

In certain embodiments, the inhibitor or antagonist of human OR10H1, or a paralogue, orthologue or other variant thereof; and the inhibitor or antagonist of Cx32, CSK and/or PKA is administered concomitantly during said treatment; and in other embodiments they are treated sequentially, such as between about 1 hour, 3 hours, 6 hours, 8 hours, 12 hours, 1 day, 2 days, 3 days, 5 days, 1 week, two weeks or about one month of each other, in particular between about 1 day and about two weeks of each other, preferably between about 2 days and about one week of each other.

In a related aspect, the invention also relates to a combination of (a) an inhibitor or antagonist of human OR10H1, or a paralogue, orthologue or other variant thereof; and (b) an inhibitor or antagonist of Cx32, CSK and/or PKA, in particular a combination for use in a combination method of treatment as described herein. A combination of (a) and (b) may be in the form of a co-formulation of the two components, or may be in the form of a kit of separately packaged components.

In certain embodiments of these combination aspects, the inhibitor or antagonist of said OR10H1 or variant is, preferably, an inhibitory or antagonistic ABP of the present invention. In other embodiments of these combination aspects, said inhibitor or antagonist of said OR10H1 or variant, said inhibitor or antagonist of Cx32 and/or said inhibitor or antagonist of CSK, or said activator or agonist of PKA, is a compound selected from a polypeptide, peptide, glycoprotein, a peptidomimetic, an antibody or antibody-like molecule; a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a targeted gene editing construct, such as a CRISPRICas9 construct, a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, small cell-permeable molecules, and peptidomimetic compounds. Preferably, said inhibitor or antagonist of Cx32 and/or said inhibitor or antagonist of CSK, or said activator or agonist of PKA is a small molecule, such as one having a molecular weight of between about 50 and 500 Da,

In some embodiments of the medical uses and methods of treatment provided herein, the medical use or treatment comprises aiding a cell-mediated immune response, such as a T-cell mediated immune response in said subject.

For example, in some embodiments of the medical uses and methods of treatment provided herein, the medical use or treatment comprises a transfer of cells to said subject; such as the (host) cells of the invention and/or of immune cells (in particular those of the subject patient of those that are HLA-matched). In particular of such embodiments, the transfer of cells comprises adoptive cell transfer, such as adoptive immune cell (e.g. T cell, such as CTL) transfer.

Adoptive cell transfer (ACT) is the transfer of cells into the subject. The cells may have originated from the subject or from another individual. The cells are most commonly derived from the immune system, with the goal of improving immune functionality and characteristics. In cancer immunotherapy, T-cells can be extracted from the patient, optionally genetically modified and cultured in vitro and returned to the same subject. Accordingly, certain of such embodiments include those treatments that comprise adoptive (e.g. autologous) T-cell transfer and/or adoptive transfer of (e.g. autologous) tumour infiltrating lymphocytes (TIL) in particular in the treatment of advanced solid tumours, including melanoma and colorectal carcinoma.

In preferred embodiments of the treatment methods, the ABP (such as one that is an inhibitor or antagonist of expression, function, activity and/or stability of said OR10H1, paralogue, orthologue or other variant thereof), or an NAC encoding said ABP or the (host) cells of the invention, (or otherwise an inhibitor or antagonist of human OR10H1, or a paralogue, orthologue or other variant thereof), when administered to the subject, decreases or reduces the resistance of cells (such as tumour cells) that express OR10H1, or the variant of OR10H1, to an immune response. In other certain preferred embodiments of the invention, the ABP (such as one that is an inhibitor or antagonist of expression, function, activity and/or stability of said OR10H1, paralogue, orthologue or other variant thereof), or an NAC encoding said ABP, enhances or increases the sensitivity of cells (such as tumour cells) that express OR10H1, or the variant of OR10H1, to an immune response.

The immune response, is, in particular of such embodiments, a cell-mediated immune response such as one mediated by T-cells including cytotoxic T-cells and/or TILs; and/or the immune response is the lysis and/or killing of the cells that express OR10H1 (or a paralogue, orthologue or other variant of OR10H1) that is mediated by cytotoxic T-cells and/or TILs. In other particular of such embodiments, the immune response is a cytotoxic immune response against cells (such as tumour cells) that express OR10H1 (or a paralogue, orthologue or other variant of OR10H1), in particular a cell-mediated cytotoxic immune response such as one mediated by T-cells including cytotoxic T-cells and/or TILs.

Specifically, in certain preferred embodiments of such treatment methods the ABP (such as one that is an inhibitor or antagonist of expression, function, activity and/or stability of said OR10H1, paralogue, orthologue or other variant thereof), or an NAC encoding said ABP or the (host) cells of the invention, (or otherwise an inhibitor or antagonist of human OR10H1, or a paralogue, orthologue or other variant thereof), when administered to the subject, enhances or increases killing and/or lysis of cells expressing OR10H1, or the variant of OR10H1, (such as tumour cells); preferably killing and/or lysis being mediated by cytotoxic T-cells and/or TILs, and/or mediated by an enhancement of or increase in the sensitivity of the cells expressing OR10H1 (or the variant of OR10H1) to a (cytotoxic) immune response, such an immune response described above, and/or mediated by a decrease in or reduction of the resistance of the cells expressing OR10H1 (or the variant of OR10H1) to a (cytotoxic) immune response, such an immune response described above.

The cells that express OR10H1 (or a paralogue, orthologue or other variant of OR10H1) are, in certain of such preferred embodiments, cancer cells or are cells that originated from a tumor cell. Exemplary cancer or tumor cells can be those as described or exemplified elsewhere herein.

In other certain preferred embodiments of such treatment methods, the ABP (such as one that is an inhibitor or antagonist of expression, function, activity and/or stability of said OR10H1, paralogue, orthologue or other variant thereof), or an NAC encoding said ABP or the (host) cells of the invention, (or otherwise an inhibitor or antagonist of human OR10H1, or a paralogue, orthologue or other variant thereof), when administered to the subject, increases T-cell activity and/or survival, which in certain embodiments, may lead to an enhancement of a (cytotoxic) immune response mediated by such T-cells.

Diagnostic Methods

The ABPs of the invention can be used for diagnostic purposes to detect, diagnose, or monitor diseases and/or conditions associated with human OR10H1 expression and/or activity, or that of a paralogue, orthologue or other variant thereof; and in particular aberrant and/or localised expression/activity of the OR10H1 or variant. In preferred embodiments of the detection and diagnosis methods of the invention, the disease, disorder or condition is a proliferative disorder (such as a tumour or cancer), or an infection, or immune deficiency; more preferably one or more of the proliferative disorders described elsewhere herein, such as melanoma, pancreatic cancer and/or colorectal cancer.

Accordingly, in another aspect, herein provided is a method (eg, an in-vitro method) of detecting a human OR10H1 or a paralogue, orthologue or other variant thereof in a sample, comprising contacting the sample with an ABP as described above and detecting binding between said ABP and said OR10H1 or variant (in said sample). If such binding is detected, then the presence of, or an amount of, said OR10H1 or variant is thereby detected or determined. Preferably, said method is conducted quantitatively, such that the amount of said OR10H1 or variant can be determined, estimated or calculated. Correspondingly, if no such binding is detected, then the absence of said OR10H1 or variant is determined (or it is determined that an amount of said OR10H1 or variant is lower than the detection threshold of the method).

In certain embodiments, the sample is a (provided) biological sample, such as one obtained from a mammalian subject like a human patient. The term “biological sample” is used in its broadest sense and can refer to a bodily sample obtained from the subject (e.g., a human patient). For example, the biological sample can include a clinical sample, i.e., a sample derived from a subject. Such samples can include, but are not limited to: peripheral bodily fluids, which may or may not contain cells, e.g., blood, urine, plasma, mucous, bile pancreatic juice, supernatant fluid, and serum; tissue or fine needle biopsy samples; tumour biopsy samples or sections (or cells thereof), and archival samples with known diagnosis, treatment and/or outcome history. Biological samples may also include sections of tissues, such as frozen sections taken for histological purposes. The term “biological sample” can also encompass any material derived by processing the sample. Derived materials can include, but are not limited to, cells (or their progeny) isolated from the biological sample, nucleic acids and/or proteins extracted from the sample. Processing of the biological sample may involve one or more of, filtration, distillation, extraction, concentration, fixation, inactivation of interfering components, addition of reagents, and the like.

Preferably, the sample is a (provided) biological sample (that had been) obtained from a mammalian subject, such as a human, and said sample comprises cells or tissue of said subject, or an extract of said cells or tissue. In a further such embodiment, the method of detecting said OR10H1 or variant further comprises the step of determining whether said mammalian subject has a phenotype associated with (e.g. a disease, disorder or condition associated with) cellular resistance against a cell-mediated immune response, wherein the presence or amount of said OR10H1 or other variant thereof in said sample indicates a phenotype associated with cellular resistance against the cell-mediated immune response in said subject.

In some embodiments, this method is a computer-implemented method, or one that is assisted or supported by a computer. In some embodiments, information reflecting the presence or an amount of a human OR10H1 or a paralogue, orthologue or other variant thereof in a sample is obtained by at least one processor, and/or information reflecting the presence or an amount of a human OR10H1 or a paralogue, orthologue or other variant thereof in a sample is provided in user readable format by another processor. The one or more processors may be coupled to random access memory operating under control of or in conjunction with a computer operating system. The processors may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processors may communicate with data storage devices, such as a database stored on a hard drive or drive array, to access or store program instructions other data. Processors may further communicate via a network interface, which in turn may communicate via the one or more networks, such as the Internet or other public or private networks, such that a query or other request may be received from a client, or other device or service. In some embodiments, the computer-implemented method of detecting the presence or an amount of a human OR10H1 or a paralogue, orthologue or other variant thereof in a sample is provided as a kit.

In yet another aspect, herein provided is a method of determining whether a mammalian subject, such as a human, has or is at risk of developing a phenotype associated with (e.g. a disease, disorder or condition associated with) cellular resistance against a cell-mediated immune response, comprising the steps of:
(i) providing (such as by obtaining) a biological sample from said subject, said sample comprising cells or tissue of said subject, or an extract of said cells or tissue; and
(ii) detecting a human OR10H1 or a paralogue, orthologue or other variant thereof in said sample, comprising contacting the sample with an ABP as described above and detecting binding between said ABP and said OR10H1 or variant;

wherein the detection of a human OR10H1 or paralogue, orthologue or other variant thereof in said sample indicates a phenotype associated with cellular resistance against the cell-mediated immune response in said subject. Said detection may comprise measuring the presence or an amount of a human OR10H1 or paralogue, orthologue or other variant thereof in said sample.

In a variant of such method, the method comprises contacting a biological sample from said subject, said sample comprising cells or tissue of said subject, or an extract of said cells or tissue, with an ABP of the invention;

wherein detection of a human OR10H1 or paralogue, orthologue or other variant thereof in said sample indicates a phenotype associated with cellular resistance against the cell-mediated immune response in said subject. Said detection may comprise measuring the presence or an amount of a human OR10H1 or paralogue, orthologue or other variant thereof in said sample.

In certain embodiments, the resistance against a cell-mediated immune response is cellular resistance against a T-cell immune response.

In particular embodiments, such detection and/or determination methods can be practiced as a method or diagnosis, such as a method of diagnosis whether a mammalian subject (such as a human subject or patient) has a disease, disorder or condition associated with cellular resistance against a cell-mediated immune response; in particular a cancer, such as one having cellular resistance against a T-cell immune response.

The antigen to be detected in the detection, determination and diagnosis methods of the invention can comprise any of the epitopes of OR10H1 as described above.

Such detection, determination and diagnosis methods can be conducted as an in-vitro method, and can be, for example, practiced using the kit of the present invention (or components thereof).

In some embodiments of said methods, the biological sample is a tissue sample from said subject, such as a sample of a tumour or a cancer from said subject. As described above, such tissue sample may be a biopsy sample of the tumour or a cancer such as a needle biopsy samples, or a tumour biopsy sections or an archival sample thereof. Such a tissue sample may comprise living, dead or fixed cells, such as from the tumour or a cancer, and such cells may be suspected of expressing (e.g. aberrantly or localised) said OR10H1 or variant.

In some embodiments, determination and/or diagnosis methods of the invention can comprise, such as in a further step, comparing detected amount of said OR10H1 or variant with a standard or cut-off value; wherein a detected amount greater than the standard or cut-off value indicates a phenotype associated with cellular resistance against the cell-mediated immune response in said subject. Such a standard or cut-off value may be determined from the use of a control assay, or may be pre-determined from one or more values obtained from a study or a plurality of samples having known phenotypes. For example, a cut-off value for a diagnostic test may be determined by the analysis of samples taken from patients in the context of a controlled clinical study, and determination of a cut-off depending on the desired (or obtained) sensitivity and/or specificity of the test.

Examples of methods useful in the detection of (such as the presence or absence of, or an amount of) said OR10H1 or variant include immunoassays, such as the enzyme linked immunosorbent assay (ELISA) and the radioimmunoassay (RIA), which employ ABPs of the invention.

For such detection, determination or diagnostic applications, the ABP typically will be labelled with a detectable labelling group. Suitable labelling groups include, but are not limited to, the following: radioisotopes or radionuclides (e.g., 3H, ¹⁴C, ¹⁵N, ³⁵S, ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labelling group is coupled to the ABP via spacer arms of various lengths to reduce potential steric hindrance. Various methods for labelling proteins are known in the art and may be used. For example, the ABP may be labelled with a secondary reporter (e.g., leucine zipper pair sequences, binding sites for secondary antibodies, metal binding domains, epitope tags, etc.).

In another aspect, the invention relates to a method to determine (or diagnose) whether a mammalian subject (such as a human subject or patient) has a disease, disorder or condition associated with cellular resistance against a cell-mediated immune response; in particular a cancer, such as one having cellular resistance against a cell-mediated immune response, such as a T cell-mediated immune response; wherein said determination/diagnosis is made with the involvement of a functional assay measuring a biological response, such as one described elsewhere herein. For example, one such method can comprise the steps of:
(i) providing (such as by obtaining) a biological sample from said subject, said sample comprising cells of said subject (such as cells of a tumour or cancer of said subject); and
(ii) contacting said cells with an ABP of the invention in the presence of immune cells selected from the group consisting of: lymphocytes, T-cells, CTLs and TILs; and
(iii) determining the cell-mediated immune response between said cells of the sample and said immune cells;

wherein an enhancement of the cell-mediated immune response indicates a phenotype associated with cellular resistance against the cell-mediated immune response in said subject.

The enhancement of the cell-mediated immune response can be determined by, for example, increased cytotoxicity of the cells of the sample (eg, increased lysis and/or killing of the cells that express OR10H1 (or a paralogue, orthologue or other variant of OR10H1), such as that which is mediated by cytotoxic T-cells and/or TILs), increased activity and/or survival of the immune cells, increased type-I cytokine secretion by said immune cells, reduced phosphorylation of CREB in said immune cells and/or increased activity (e.g. by reduced phosphorylation of Tyr505) (and/or reduced level of cAMP and/or phosphorylation of PKA) in said immune cells. Methods to determine such biological responses are described elsewhere herein, for example including the chromium-release cytotoxicity assay described above.

In another aspect, the invention relates to a method of diagnosing and treating a disease, disorder or condition characterised by expression of a human OR10H1, or a paralogue, orthologue or other variant thereof (such as a proliferative disorder for example a tumour or cancer, and/or an infection and/or an immune disorder such as an immune deficiency) in a mammalian subject, such as a human patient, comprising:
(i) providing (such as by obtaining) a biological sample from said subject, said sample comprising cells or tissue of said subject, or an extract of said cells or tissue; and
(ii) detecting whether said OR10H1 or variant is present in the biological sample;
(iii) diagnosing the subject with the disease, disorder or condition depending on if the presence of said OR10H1 or variant in the biological sample is detected; and
(iv) administering an effective amount of the inventive ABP, NAC, cells and/or pharmaceutical composition to the diagnosed subject.

In some embodiments of such a method of diagnosis and treatment, the presence of said OR10H1 or variant may be directly detected using a method of detection as set out herein (for example, utilising an ABP of the invention). Alternatively, the presence of said OR10H1 or variant may be detected indirectly, for example by detection of the presence of mRNA that encoding said OR10H1 or variant, or fragments of such mRNA. Methods to detect the presence of such mRNA (or fragments) can include, PCR (such as quantitative RT-PCR), hybridisation (such as to Illumina chips), nucleic-acid sequencing etc. Such methods may involve or comprise steps using one or more nucleic acids or NACs of the invention.

Further embodiments of the administering step of this method of diagnosis and treatment are described in more details above.

The detection, determination and/or diagnosis methods of the present invention, may further comprise, such as in a further step, detecting the presence of Cx32 in the sample (or an additional sample); wherein the presence of Cx32 in said sample (for example, expressed by cells of a tumour) indicates that a patient from which the sample obtained may be suitable for (e.g. selected for) treatment with an ABP of the present invention, and/or with another inhibitor or antagonist of human OR10H1, or a paralogue, orthologue or other variant thereof.

In yet another aspect, the invention relates to an ABP, nucleic acid or NAC of the invention, for use in diagnosis, such as in the detection of a disease, disorder or condition in a mammalian subject, such as a human patient, in particular of a disease, disorder or condition associated with cellular resistance against a cell-mediated immune response (such as a cancer).

In certain embodiments, the detection/diagnostic methods of the invention involve an immunohistochemistry (IHC) assay or an immunocytochemistry (IC) assay. The terms “IHC” and “ICC” are art recognised, and include the meanings of techniques employed to localise antigen expression that are dependent on specific epitope-antibody interactions. IHC typically refers to the use of tissue sections, whereas ICC typically describes the use of cultured cells or cell suspensions. In both methods, positive staining is typically visualised using a molecular label (e.g., one which may be fluorescent or chromogenic). Briefly, samples are typically fixed to preserve cellular integrity, and then subjected to incubation with blocking reagents to prevent non-specific binding of the antibodies. Samples are subsequently typically incubated with primary (and sometimes secondary) antibodies, and the signal is visualised for microscopic analysis.

Accordingly, such embodiments of the detection/diagnostic methods of the invention may include a step of preparing a subject IHC or ICC preparation tissue or cells (such as those present in the biological samples obtained from a subject); and preferably wherein the detection of binding of an ABP of the invention to OR10H1 or a variant thereof expressed by the tissues of cells said IHC or ICC preparation indicates: (i) a phenotype associated with cellular resistance against the cell-mediated immune response in said subject; and/or (ii) said subject has or has a risk of developing disease, condition or disorder associated with (aberrant) expression of OR10H1 or said variant.

Preferably, in such IHC/ICC methods is used an ABP of the invention that does not bind (e.g. does not detectably bind) to a validation IHC or ICC preparation of mammalian tissues or cells other than to (detectably) bind to OR10H1, or a paralogue, orthologue or other variant thereof, that is expressed by the mammalian tissue cells or of said validation IHC or ICC preparation.

In certain of such embodiment, said validation and/or subject IHC or ICC preparation is one selected from the list consisting of: a frozen section, a paraffin section, and a resin section, in each case of the tissues and/or cells; and/or wherein the tissues and/or cell comprised in either (or both) said IHC or ICC preparations are fixed. The tissues and/or cells or such IHC or ICC preparation(s) may be fixed by an alcohol, an aldehyde, a mercurial agent, an oxidising agent or a picrate.

In one preferred of such embodiments, said validation and/or subject IHC or ICC preparation is a formalin-fixed paraffin embedded (FFPE) section of said tissues and/or cells; and/or wherein said validation and/or subject IHC or ICC preparation is subjected to antigen retrieval (AR). Such AR may comprise protease-induced epitope retrieval (PIER) or heat-induced epitope retrieval (HIER).

The ABP used in such methods is, preferably, validated. For example, the ABP is validated to (detectably) bind to said OR10H1 or variant expressed by the cells and/or tissues of said validation IHC or ICC preparation, but does not (detectably) bind to a control IHC or ICC preparation of control cells and/or tissues that do not express said OR10H1 or variant. Preferably, said control cells are gene knock-down or gene knock-out cells and/or tissues for said OR10H1 or variant; more preferably, wherein said gene knock-down or gene knock-out cells and/or tissues are siRNA or shRNA gene knock-down or gene knock-out for said OR10H1 or variant. Such control cells may comprise cells from a cell-line selected from the list consisting of KMM-1 and said control cells and/or tissues that do not express said OR10H1 or variant comprise cells of said cell line that have been transfected with a siRNA or shRNA selected from those on Tables El or E2; and/or said validation IHC or ICC preparation comprises cells from M579-A2 cells.

In such IHC/ICC methods, the ABP is used with said validation and/or subject IHC or ICC preparation at a working concentration of less than about 50ug/mL, 25ug/mL, 20ug/mL, 15ug/mL, 10 ug/mL, 7.5 μg/mL, 5 μg/mL, 2.5 μg/mL, 1 μg/mL, 0.5 μg/mL, 0.2 μg/ml or 0.1 μg/ml, in particular less than about 5 μg/mL, and more particularly at less than 2.5 μg/mL; preferably, at a concentration that is about 2-fold, 5-fold, 10-fold, 20-fold or 50-fold higher than said working concentration, said ABP does not (detectably) bind to said validation immunohistochemistry (IHC) preparation of mammalian cells or tissues other than to [detectably] bind to said OR10H1 or variant thereof expressed by the mammalian cells or tissue of said IHC preparation, in particular at a concentration that is about 2-fold higher than said working concentration, and more particularly at a concentration that is about 5-fold higher than said working concentration.

In the detection/diagnostic methods of the invention, the ABP used may be a polyclonal antibody; and preferably may be a rabbit antibody.

As will now be apparent, such detection, determination and/or diagnosis methods may further comprise or utilise any of the additional steps or embodiments as may be used or applicable in the above method of detecting the presence or an amount of a human OR10H1 or a paralogue, orthologue or other variant thereof in a sample. For example, in certain embodiments, the method of determination (or diagnosis) of the invention is a computer-implemented method.

### Diagnostic Kits

In another aspect, herein provided is a kit for performing the diagnostic methods or detection methods of the invention, e.g., for determining the presence, absence, amount, function, activity and/or expression of a human OR10H1 or a paralogue, orthologue or other variant thereof in a sample (e.g. a biological sample), such as on cells in a sample. The kit comprises an ABP, a nucleic acid and/or a NAC as described above and, optionally one or more additional components.

In certain embodiments of the kit, an additional component may comprise instructions describing how to use the ABP, a nucleic acid and/or a NAC, or kit, for detecting the presence of said ABP in said sample, such as by detecting binding between said ABP and said OR10H1 or variant. Such instructions may consist of a printed manual or computer readable memory comprising such instructions, or may comprise instructions as to identify, obtain and/or use one or more other components to be used together with the kit.

In other certain embodiments of the kit, the additional component may comprise one or more other item, moiety, component, reagent, detector or other means useful for the use of the kit or practice of a detection method of the invention, including any such item, moiety, component, reagent, detector or (other) means disclosed herein useful for such practice. For example, the kit may further comprise reaction and/or binding buffers, labels, enzymatic substrates, secondary antibodies and control samples, materials or moieties etc.

In a particular such embodiment, the additional component may comprise a detector and/or (other) means of detecting the presence of OR10H1 or variant in said sample, such as detecting binding between said ABP and said OR10H1 or variant.

Various a detector and/or (other) means for indicating the binding of an ABP can be used. For example, fluorophores, other molecular probes, or enzymes can be linked to the ABP and the presence of the ABP can be observed in a variety of ways. A method for screening for diseases or disorders can involve the use of the kit, or simply the use of one of the disclosed ABPs and the determination of the extent to which ABP binds to the human OR10H1 (or paralogue, orthologue or other variant thereof) in a sample. As will be appreciated by one of skill in the art, high or elevated levels of said OR10H1 will result in larger amounts of the ABP binding thereto in the sample. Thus, degree of ABP binding can be used to determine how much of said OR10H1 is in a sample. Subjects or samples with an amount of said OR10H1 that is greater than a predetermined amount (e.g., an amount or range that a person without a OR10H1-related disorder would have) can be characterized as having a OR10H1-mediated disorder.

In some embodiments, the kit further comprises one or more of the following: standards of OR10H1 protein, positive and/or negative controls for ABP binding, a vessel for collecting a sample, materials for detecting binding of said ABP to said OR10H1 or variant in said sample, and reagent(s) for performing said detection.

In another aspect, herein provided is the use of a kit as described above for performing the diagnostic or in vitro detection methods of the invention.

As described above, kits of the invention may be accompanied by instructions, including those to use them for determining the amount, activity and/or expression of a human OR10H1 or a paralogue, orthologue or other variant thereof in a sample, such as on T cells in a sample.

Cells and Methods of Producing the ABPs/NACs of the Invention

In another aspect, herein provided is a cell, which is a hybridoma capable of expressing an ABP as described above. In an alternative aspect, herein provided is a cell, which comprises at least one NAC encoding an ABP or a component of an ABP as described above. Cells of the invention can be used in methods provided herein to produce the ABPs and/or NACs of the invention.

In certain embodiments, the cell is isolated or substantially pure, and/or is a recombinant cell and/or is a non-natural cell (i.e., is it not found in, or is a product of, nature), such as a hybridoma.

In yet another aspect, herein provided is a method of producing an ABP as described above, comprising culturing one or more cells of the invention under conditions allowing the expression of said ABP.

For producing the recombinant ABPs of the invention, the DNA molecules encoding the proteins (e.g. for antibodies, light and/or heavy chains or fragments thereof) are inserted into an expression vector (or NAC) such that the sequences are operatively linked to transcriptional and translational control sequences. Alternatively, DNA molecules encoding the ABP can be chemically synthesized. Synthetic DNA molecules can be ligated to other appropriate nucleotide sequences, including, e.g., constant region coding sequences, and expression control sequences, to produce conventional gene expression constructs encoding the desired ABP. For manufacturing the ABPs of the invention, the skilled artisan may choose from a great variety of expression systems well known in the art, e.g. those reviewed by Kipriyanow and Le Gall, 2004. Expression vectors include plasmids, retroviruses, cosmids, EBV-derived episomes, and the like. The term “expression vector” or “NAC” comprises any vector suitable for the expression of a foreign DNA. Examples of such expression vectors are viral vectors, such as adenovirus, vaccinia virus, baculovirus and adeno-associated virus vectors. In this connection, the expression “virus vector” is understood to mean both a DNA and a viral particle. Examples of phage or cosmid vectors include pWE15, M13, λEMBL3, λEMBL4, λFIXII, λDASHII, λZAPII, λgT10, λgt11, Charon4A and Charon21A. Examples of plasmid vectors include pBR, pUC, pBluescriptII, pGEM, pTZ and pET groups. Various shuttle vectors may be used, e.g., vectors which may autonomously replicate in a plurality of host microorganisms such as E. coli and Pseudomonas sp. In addition, artificial chromosome vectors are considered as expression vectors. The expression vector and expression control sequences are selected to be compatible with the cell, such as a host cell. Examples of mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRepS, D H26S, D HBB, pNMT1, pNMT41, pNMT81, which are available from Invitrogen™, pCI which is available from Promega, pMbac, pPbac, pBK-RSV and pBK-CMV which are available from Agilent Technologies, pTRES which is available from Clontech, and their derivatives.

For manufacturing antibodies, the antibody light chain gene and the antibody heavy chain gene can be inserted into separate vectors. In certain embodiments, both DNA sequences are inserted into the same expression vector. Convenient vectors are those that encode a functionally complete human CH or CL immunoglobulin sequence, with appropriate restriction sites engineered so that any VH or VL sequence can be easily inserted and expressed, as described above, wherein the CH1 and/or upper hinge region comprises at least one amino acid modification of the invention. The constant chain is usually kappa or lambda for the antibody light chain. The recombinant expression vector may also encode a signal peptide that facilitates secretion of the antibody chain from a (host) cell. The DNA encoding the antibody chain may be cloned into the vector such that the signal peptide is linked in-frame to the amino terminus of the mature antibody chain DNA. The signal peptide may be an immunoglobulin signal peptide or a heterologous peptide from a non-immunoglobulin protein. Alternatively, the DNA sequence encoding the antibody chain may already contain a signal peptide sequence.

In addition to the DNA sequences encoding the ABP (antibody) chains, the recombinant expression vectors carry regulatory sequences including promoters, enhancers, termination and polyadenylation signals and other expression control elements that control the expression of the antibody chains in a (host) cell. Examples for promoter sequences (exemplified for expression in mammalian cells) are promoters and/or enhancers derived from CMV (such as the CMV Simian Virus 40 (SV40) promoter/enhancer), adenovirus, (e.g., the adenovirus major late promoter (AdMLP)), polyoma and strong mammalian promoters such as native immunoglobulin and actin promoters. Examples for polyadenylation signals are BGH polyA, SV40 late or early polyA; alternatively, 3′UTRs of immunoglobulin genes etc. can be used.

The recombinant expression vectors may also carry sequences that regulate replication of the vector in (host) cells (e.g. origins of replication) and selectable marker genes. Nucleic acid molecules encoding the heavy chain or an antigen-binding portion thereof and/or the light chain or an antigen-binding portion thereof of an antibody of the present invention, and vectors comprising these DNA molecules can be introduced into (host) cells, e.g. bacterial cells or higher eukaryotic cells, e.g. mammalian cells, according to transfection methods well known in the art, including liposome-mediated transfection, polycation-mediated transfection, protoplast fusion, microinjections, calcium phosphate precipitation, electroporation or transfer by viral vectors.

For antibodies or fragments thereof, it is within ordinary skill in the art to express the heavy chain and the light chain from a single expression vector or from two separate expression vectors. Preferably, the DNA molecules encoding the heavy chain and the light chain are present on two vectors which are co-transfected into the (host) cell, preferably a mammalian cell.

Mammalian cell lines available as hosts for expression are well known in the art and include, inter alia, Chinese hamster ovary (CHO, CHO-DG44, BI-HEX-CHO) cells, NSO, SP2/0 cells, HeLa cells, HEK293 cells, baby hamster kidney (BHK) cells, monkey kidney cells (COS), human carcinoma cells (e.g., Hep G2), A549 cells, 3T3 cells or the derivatives/progenies of any such cell line. Other mammalian cells, including but not limited to human, mice, rat, monkey and rodent cells lines, or other eukaryotic cells, including but not limited to yeast, insect and plant cells, or prokaryotic cells such as bacteria may be used. The antibody molecules of the invention are produced by culturing the host cells for a period of time sufficient to allow for expression of the antibody molecule in the host cells.

According to some embodiments of the method of producing an ABP, following expression, the intact antibody (or the antigen-binding fragment of the antibody) can be harvested and isolated using purification techniques well known in the art, e.g., Protein A, Protein G, affinity tags such as glutathione-S-transferase (GST) and histidine tags.

ABPs are preferably recovered from the culture medium as a secreted polypeptide or can be recovered from host cell lysates if for example expressed without a secretory signal. It is necessary to purify the ABP molecules using standard protein purification methods used for recombinant proteins and host cell proteins in a way that substantially homogenous preparations of the ABP are obtained. By way of example, state-of-the art purification methods useful for obtaining the ABP molecule of the invention include, as a first step, removal of cells and/or particulate cell debris from the culture medium or lysate. The ABP is then purified from contaminant soluble proteins, polypeptides and nucleic acids, for example, by fractionation on immunoaffinity or ion-exchange columns, ethanol precipitation, reverse phase HPLC, Sephadex chromatography, chromatography on silica or on a cation exchange resin. Preferably, ABPs are purified by standard protein A chromatography, e.g., using protein A spin columns (GE Healthcare). Protein purity may be verified by reducing SDS PAGE. ABP concentrations may be determined by measuring absorbance at 280nm and utilizing the protein specific extinction coefficient. As a final step in the process for obtaining an ABP molecule preparation, the purified ABP molecule may be dried, e.g. lyophilized, for therapeutic applications.

In another aspect, herein provided is a method of manufacturing a pharmaceutical composition comprising an ABP as described above, comprising formulating the ABP isolated by the methods described above into a pharmaceutically acceptable form.

In an alternative aspect, herein provided is a method of manufacturing a pharmaceutical composition comprising an NAC as described above, comprising formulating the NAC prepared by the methods described above into a pharmaceutically acceptable form.

According to some embodiments, the methods of manufacturing a pharmaceutical composition comprise a further step of combining said ABP and/or NAC with a pharmaceutically acceptable excipient or carrier.

In some embodiments of the method of manufacturing a pharmaceutical composition comprising an ABP, the ABP typically will be labelled with a detectable labelling group before being formulated into a pharmaceutically acceptable form. Various methods for labelling proteins are known in the art and may be used. Suitable labelling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵In, ¹³¹I), fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labelling group is coupled to the ABP via spacer arms of various lengths to reduce potential steric hindrance.

Screening Assays and Methods

In another aspect, herein provided is a method for identifying (or characterising) a compound suitable for the treatment of a disease characterized by expression (such by aberrant expression) of OR10H1 or a paralogue, orthologue or other variant thereof, the method comprising the steps of:
(a) Providing a first cell expressing a protein of OR10H1 (or of a paralogue, orthologue or other variant thereof) on the surface of the first cell, and
(b) Providing a candidate compound, and
(c) Optionally, providing a second cell which is a cytotoxic immune cell, for example a cytotoxic T-lymphocyte (CTL), capable of immunologically recognizing said first cell, and
(d) Bringing into contact the first cell and the candidate compound, and optionally the second cell, and
(e) Determining subsequent to step (d), the activity of said OR10H1 (or variant thereof) of the first cell.

wherein, a reduced (or increased) activity of said OR10H1 (or variant thereof) of the first cell contacted with the candidate compound (in particular, compared to a said first cell not contacted with said candidate compound) indicates that the candidate compound is a compound suitable for the treatment of a disease characterized by expression (such by aberrant expression) of OR10H1 or a paralogue, orthologue or other variant thereof.

A reduction in activity of said OR10H1 (or variant thereof) of the first cell contacted with the candidate compound can identify those compounds that are inhibitors or antagonists of expression, function, activity and/or stability of said OR10H1 or variant; preferably a compound that is an inhibitor or antagonist of the function and/or activity of said OR10H1 or variant.

Corresponding, an increase in activity of said OR10H1 (or variant thereof) of the first cell contacted with the candidate compound can identify those compounds that are activators or agonists of expression, function, activity and/or stability of said OR10H1 or variant; preferably a compound that is an activator or agonist of the function and/or activity of said OR10H1 or variant.

The OR10H1 (or of a paralogue, orthologue or other variant thereof) expressed on the surface of the cell, and/or the signal transduction pathway thereof, may, in certain embodiments, be constitutively active (or activated). Accordingly, the method may include: (i) the use of a constitutively active mutant of OR10H1 (or variant thereof), such as those mutants that may be analogously obtained from the method and disclosure in WO 00/22131 and/or WO 01/27632; and/or (ii) a step of contacting the cell with an agent that stimulate the signal transduction pathway, such as forskolin (eg at between about 1 and 50 uM such as between about 5 and 20 uM or, in particular, about 10 uM forskilin).

In particular embodiments of such method, the activity of said OR10H1 (or variant thereof) of the first cell may be determined (or otherwise measured or estimated) from the amount of cAMP produced by the first cell.

The amount of cAMP produced by the first cell may be determined by any suitable method. For example, in particular embodiments of such method, the first cell expresses a detectable protein, the detectable amount or signal thereof is positively correlated to the amount of cAMP produced by the first cell. Such detectable protein may be, in such embodiments, a luminescent or fluorescent protein, such as luciferase or GFP (or variant thereof), in particular one under the control of a cAMP response element (CRE), such as cre-reporter constructs known as Csignal kits from Qiagen (Hildon, Germany).

The amount of cAMP may be determined in the presence of (eg, the method may include the step of exposing the cell to) an inhibitor of phosphodiesterase (PDE), such as isobutyl-1-methylxanthine (IBMX).

The reduction (or enhancement) of expression, function and/or stability or OR10H1 (or of the variant thereof), or the enhancement (or reduction) in cytotoxicity is, preferably, identified by reference to a control method, for example one practiced in the absence of any candidate compound, or with compound having a know effect on such expression, function and/or stability (such as a positive or negative control). Alternatively (or in addition), the activity/signals obtained from such cell contacted with the candidate compound may be compared to an activity/signal obtained from a different cell contacted with the candidate compound, such as a different cell that does not express said OR10H1 (or the variant) and/or does not signal via the G-alpha-Olf/S pathway, in particular a recombinant cell that expresses an exogenous G protein coupled receptor (GPCR) that is not OR10H1 (eg, that expresses GPR41).

The first cell may be an immortalized cell line, such as HEK293 or modifications thereof, and/or said first cell may be a tumour cell, or a cell derived from a tumour cell. In particular, the first cell may be a recombinant cell, such as one expressing an exogenous recombinant OR10H1 (or paralogue, orthologue or other variant thereof).

The candidate compound may be one selected from a polypeptide, peptide, glycoprotein, a peptidomimetic, an antigen binding construct (for example, an antibody, antibody-like molecule or other antigen binding derivative, or an or antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA), a genetic construct for targeted gene editing, such as a CRISPR/Cas9 construct and/or a guide nucleic acid (gRNA or gDNA) and/or tracrRNA.

In particular embodiments of such screening method, the candidate compound is a small molecule. Typically, a small molecule is a compound having a molecular mass of less than about 750 Da, such as less than about 650 or 600 Da, (and in certain embodiments, a small molecule may be less than about 550 or 500 Da). Furthermore, (in particularly for a cell-permeable compound, and especially for an orally active compound), the small molecule can have, in certain embodiments: (i) no more than 5 hydrogen bond donors (the total number of nitrogen-hydrogen and oxygen-hydrogen bonds); (ii) no more than 10 hydrogen bond acceptors (all nitrogen or oxygen atoms); and/or (iii) an octanol-water partition coefficient log P not greater than 5. Accordingly, in some embodiments the small molecule can be a cell-permeable small organic molecule, such as one that binds to an extracellular domain of OR10H1 or a paralogue, orthologue or other variant thereof, and inhibits the its function or activity, such as reduces the amount of cAMP produced by the cell expressing the OR10H1 or paralogue, orthologue or other variant.

In other particular embodiments of such screening method, the candidate compound is an ABP, such as one described elsewhere herein as being an ABP of the invention.

In certain embodiments, the candidate compound is comprised in a collection of other candidate compounds, such as a collection comprising at least about 10, 100, 1,000, 10,000 or more candidate compounds. For example, the candidate compound may be comprised in a library of candidate compounds totaling at least (or about) 20,000, 50,000, 75,000, 100,000, 150,000, 250,000 or 500,000 compounds. Compound libraries of such size are routinely available for screening of eg small molecule compounds; such as those obtainable from the Compound Cloud (BioAscent, Newhouse, Scotland).

In certain preferred embodiments of the invention, the compound—eg, one identified (or characterised) in such method—(such as a compound that is an inhibitor or antagonist of expression, function, activity and/or stability of said OR10H1, or paralogue, orthologue or other variant thereof) decreases or reduces the resistance of cells (such as tumour cells) that express OR10H1, or the variant of OR10H1, to an immune response. In other certain preferred embodiments of the invention, the compound—eg, one identified (or characterised) in such method—(such as a compound that is an inhibitor or antagonist of expression, function, activity and/or stability of said OR10H1, paralogue, orthologue or other variant thereof) enhances or increases the sensitivity of cells (such as tumour cells) that express OR10H1, or the variant of OR10H1, to an immune response.

The immune response, is, in particular of such embodiments, a cell-mediated immune response such as one mediated by T-cells including cytotoxic T-cells and/or TILs; and/or the immune response is the lysis and/or killing of the cells that express OR10H1 (or a paralogue, orthologue or other variant of OR10H1) that is mediated by cytotoxic T-cells and/or TILs. In other particular of such embodiments, the immune response is a cytotoxic immune response against cells (such as tumour cells) that express OR10H1 (or a paralogue, orthologue or other variant of OR10H1), in particular a cell-mediated cytotoxic immune response such as one mediated by T-cells including cytotoxic T-cells and/or TILs.

Specifically, in certain preferred embodiments, the compound—eg, one identified (or characterised) in such method—(such as a compound that is an inhibitor or antagonist of expression, function, activity and/or stability of said OR10H1, or paralogue, orthologue or other variant thereof) enhances or increases killing and/or lysis of cells expressing OR10H1, or the variant of OR10H1, (such as tumour cells); preferably killing and/or lysis being mediated by cytotoxic T-cells and/or TILs, and/or mediated by an enhancement of or increase in the sensitivity of the cells expressing OR10H1 (or the variant of OR10H1) to a (cytotoxic) immune response, such an immune response described above, and/or mediated by a decrease in or reduction of the resistance of the cells expressing OR10H1 (or the variant of OR10H1) to a (cytotoxic) immune response, such an immune response described above.

The cells that express OR10H1 (or a paralogue, orthologue or other variant of OR10H1) are, in certain of such preferred embodiments, cancer cells or are cells that originated from a tumor cell. Exemplary cancer or tumor cells can be those as described or exemplified elsewhere herein.

In other certain preferred embodiments of the invention, the compound—eg, one identified (or characterised) in such method—(such as a compound that is an inhibitor or antagonist of expression, function, activity and/or stability of said OR10H1, or paralogue, orthologue or other variant thereof) increases T-cell activity and/or survival, which in certain embodiments, may lead to an enhancement of a (cytotoxic) immune response mediated by such T-cells.

In view of the above, it will be appreciated that the present invention also relates to the following items:

Item 1. An antigen binding protein (ABP) that binds to a human olfactory receptor 10H1 (OR10H1), or a paralogue, orthologue or other variant thereof, wherein said ABP binds to an epitope displayed by one or more extracellular domain(s) of said OR10H1, paralogue, orthologue or other variant.

Item 2. The ABP of item 1, wherein the ABP binds to an OR10H1 variant that shares at least 95%, at least 96%, at least 97%, at least 98%, or at least 99% amino acid sequence identity with SEQ ID NO:1, or to an OR10H1 that is identical to SEQ ID NO: 1.

Item 3. The ABP of item 1, wherein the ABP binds to an OR10H1 variant that shares at least 95%, at least 96%, at least 97%, at least 98%, or at least 99% amino acid sequence identity with SEQ ID NO: 2, or to an OR10H1 that is identical to SEQ ID NO: 2.

Item 4. The ABP of item 1, wherein the ABP binds to an OR10H1 variant that shares at least 95%, at least 96%, at least 97%, at least 98%, or at least 99% amino acid sequence identity with SEQ ID NO: 3, or to an OR10H1 that is identical to SEQ ID NO: 3.

Item 5. The ABP of item 1, wherein the ABP binds to an OR10H1 variant that shares at least 95%, at least 96%, at least 97%, at least 98%, or at least 99% amino acid sequence identity with SEQ ID NO: 4, or to an OR10H1 that is identical to SEQ ID NO: 4.

Item 6. The ABP of item 1, wherein the ABP binds to an OR10H1 variant that shares at least 95%, at least 96%, at least 97%, at least 98%, or at least 99% amino acid sequence identity with SEQ ID NO: 12, or to an OR10H1 that is identical to SEQ ID NO: 12.

Item 7. The ABP of item 1, wherein the ABP binds to at least one human OR10H1 selected from the group consisting of SEQ ID NO: 1, SEQ ID NO: 2, SEQ ID NO: 3, SEQ ID NO: 4 and SEQ ID NO: 12.

Item 8. The ABP of any one of items 1 to 7, wherein the ABP binds to at least one Cynomolgus orthologue of OR10H1, such as one identical to SEQ ID NO: 11, or a variant thereof; preferably in addition to binding to at least one human OR10H1.

Item 9. The ABP of any one of items 1 to 8, which is isolated and/or substantially pure.

Item 10. The ABP of any one of items 1 to 9 which: (i) binds to said OR10H1 or variant with a KD that is less than 1 nM, is less than 100 pM, or is less than 10 pM; and/or (ii) binds to a human cell line expressing said OR10H1 or variant with an EC50 of less than 2ug/mL.

Item 11. The ABP of any one of items 1 to 10 which is an antibody, such as a monoclonal antibody.

Item 12. The ABP of any one of items 1 to 10 which is a fragment of an antibody, such as a fragment of a monoclonal antibody.

Item 13. The ABP of item 11 or 12, wherein said antibody is a chimeric antibody, such as a human-chimeric antibody.

Item 14. The ABP of any one of items 11 to 13, wherein said antibody is a humanised antibody.

Item 15. The ABP of any one of items 11 to 14, wherein said antibody is a human antibody.

Item 16. The ABP of item 12, which is a fragment of a human antibody, such as a fragment of a human monoclonal antibody.

Item 17. The ABP of any one of items 11 to 16, wherein said antibody is an IgG, IgE, IgD, IgA, or IgM immunoglobulin; preferably an IgG immunoglobulin.

Item 18. The ABP of any one of items 11 to 17, which is an antibody fragment selected from the list consisting of: Fab, Fab′-SH, Fv, scFv and F(ab′)2.

Item 19. The ABP of any one of items 11 to 18, wherein at least a portion of the framework sequence of said antibody or fragment thereof is a human consensus framework sequence.

Item 20. The ABP of any one of items 1 to 19, wherein said ABP is modified or engineered to increase ADCC, preferably wherein said ABP is afucosylated.

Item 21. The ABP of any one of items 1 to 20, which binds to (a) one or more epitopes displayed by one or more of said extracellular domains; or which binds to (b) two or more epitopes displayed by one or more of said extracellular domains.

Item 22. The ABP of any one of items 1 to 21, wherein said epitope(s) is/are displayed by two or more of said extracellular domains.

Item 23. The ABP of any one of items 1 to 22, wherein said epitope(s) is/are formed by a stretch of amino acids of between about 3 and 37 amino acids, such as between 5 and 20 or between 4 and 8 amino acids, comprised in any of the amino acid sequences independently selected from the group consisting of: SEQ ID NO:5, SEQ ID NO:6, SEQ ID NO:7, SEQ ID NO:8, SEQ ID NO:9, SEQ ID NO:10 and SEQ ID NO:13.

Item 24. The ABP of item 23, wherein said epitope(s) is/are formed by a stretch of amino acids comprised in amino acid sequence SEQ ID NO: 5 and/or 9, and wherein said ABP also binds to the cynomolgus orthologue of human OR10H1 (SEQ ID NO: 11).

Item 25. The ABP of item 23, wherein said epitope(s) is/are formed by a stretch of amino acids comprised in amino acid sequence SEQ ID NO: 6, and wherein said ABP also binds to the cynomolgus orthologue of human OR10H1 (SEQ ID NO: 11) and to OR10H2 (SEQ ID NO: 15) and/or to OR10H5 (SEQ ID NO: 14)

Item 26. The ABP of item 23, wherein said epitope(s) is/are formed by a stretch of amino acids comprised in amino acid sequence SEQ ID NO: 7, 10 and/or 13, and wherein said ABP also binds to OR10H5 (SEQ ID NO: 14).

Item 27. The ABP of item 23, wherein said epitope(s) is/are formed by a stretch of amino acids comprised in amino acid sequence SEQ ID NO: 8, and wherein said ABP also binds to the cynomolgus orthologue of human OR10H1 (SEQ ID NO: 11).

Item 28. The ABP of any one of items 1, 2, and 7 to 23, wherein each of said extracellular domain(s) is independently selected from the group consisting of: SEQ ID NO:5, SEQ ID NO:6, SEQ ID NO:7, and SEQ ID NO:8.

Item 29. The ABP of any one of items 1, 3, 4, and 7 to 23, wherein each of said extracellular domain(s) is independently selected from the group consisting of: SEQ ID NO:9, SEQ ID NO:6, SEQ ID NO:7, and SEQ ID NO:8.

Item 30. The ABP of any one of items 1, 5 and 7 to 23, wherein each of said extracellular domain(s) is independently selected from the group consisting of: SEQ ID NO:5, SEQ ID NO:6, SEQ ID NO:10, and SEQ ID NO:8.

Item 31. The ABP of any one of items 1, 6 and 7 to 23, wherein each of said extracellular domain(s) is independently selected from the group consisting of: SEQ ID NO:5, SEQ ID NO:6, SEQ ID NO:13, and SEQ ID NO:8.

Item 32. The ABP of any one of items 1 to 31, wherein at least one of said epitopes is formed by a stretch of amino acids of between about 3 and 37 amino acids, such as between 5 and 20 or between 4 and 8 amino acids, comprised in the amino acid sequence of SEQ ID NO:6.

Item 33. The ABP of any one of items 1 to 32, which additionally comprises one or more additional antigen binding domain(s) that bind(s) to antigen(s) other than said OR10H1 or variant; such as antigen(s) present on a mammalian T-cell.

Item 34. The ABP of any one of items 1 to 33, which is a modulator of the expression, function, activity and/or stability of said OR10H1 or variant.

Item 35. The ABP of item 34, which is an inhibitor or antagonist of expression, function, activity and/or stability of said OR10H1 or variant.

Item 36. The ABP of item 35, which is an inhibitor of the function and/or activity of said OR10H1 or variant.

Item 37. The ABP of item 35 or 36, which reduces the amount and/or surface concentration of said OR10H1 or variant present on the surface of a mammalian cell; preferably by ABP-induced internalisation, and optionally degradation, of said OR10H1 or variant present on the surface of the mammalian cell.

Item 38. The ABP of any one of items 35 to 37 which modulates the binding: (i) of one or more chemostimuli of OR10H1 to OR10H1 expressed on the surface of a mammalian cell; and/or (ii) of immune cells, such as T-cells, to the surface of a mammalian cell expressing said OR10H1 or variant.

Item 39. The ABP of any one of items 35 to 38 which increases the activity of immune cells, such as T-cells, including when such T-cells are bound to the surface of a mammalian cell expressing said OR10H1 or variant.

Item 40. The ABP of any one of items 35 to 39, wherein said ABP: (i) enhances a cell-mediated immune response, such as that mediated by an activated cytotoxic T-cell (CTL), to a mammalian cell expressing said OR10H1 or variant; and/or (ii) increases immune cell, such as T-cell, activity and/or survival in the presence of a mammalian cell expressing said OR10H1 or variant.

Item 41. The ABP of item 39 or 40, wherein said ABP increases type-I cytokine secretion by said immune cells, for example T-cells or CTLs, such as one or more cytokines independently selected from the list consisting of: IFN-gamma, IL-2 and TNF-alpha.

Item 42. The ABP of any one of items 1 to 41, wherein said ABP comprises an effector group. Item 43. The ABP of any one of items 1 to 42, wherein said ABP is labelled. Item 44. A nucleic acid encoding an ABP of any one of items 1 to 43 or a component of said ABP.

Item 45. A nucleic acid construct (NAC) comprising a nucleic acid of item 44 and one or more additional features permitting the expression of the encoded ABP or component of said ABP in a cell.

Item 46. A cell comprising a NAC of item 45 capable of expressing the encoded ABP or component of said ABP.

Item 47. The cell of item 46 which is a human cell, preferably an autologous human cell.

Item 48. A pharmaceutical composition comprising (a) at least one ABP of any one of items 1 to 43; (b) at least one NAC of item 45 encoding said ABP or component of said ABP; and/or (c) cells of item 46 or item 47, and a pharmaceutically acceptable excipient or carrier.

Item 49. The pharmaceutical composition of item 48, which is in unit dose form of: (i) between about 10 mg and about 1000 mg for said ABP; and/or (ii) between about 5 μg and about 5000 μg for said NAC; and/or (iii) between about 1×10\hat{ }3 and about 1×10\hat{ }9 cells of said cells.

Item 50. A method of modulating the expression, function, activity and/or stability of a human OR10H1 or paralogue, orthologue or other variant thereof comprising contacting a cell that expresses said OR10H1 or variant with an ABP of item 34, or an NAC encoding said ABP according to item 45, thereby modulating the expression, function, activity and/or stability of said OR10H1 or variant.

Item 51. A method of enhancing a cell-mediated immune response to a mammalian cell, such as a human cell, that expresses a human OR10H1 or paralogue, orthologue or other variant thereof, comprising contacting said cell with an ABP of item 34, or an NAC encoding said ABP according to item 45, in the presence of an immune cell, such as a T-cell, wherein said ABP is an inhibitor of the expression, function, activity and/or stability of said OR10H1 or variant, thereby enhancing a cell-mediated immune response.

Item 52. The ABP of any one of items 1 to 43, the NAC of item 45, cells of item 46 or item 47, or the pharmaceutical composition of item 48 or 49, for use in medicine, such as in the treatment or the prevention of a disease, disorder or condition in a mammalian subject, such as a human patient.

Item 53. A method of treating or preventing a disease, disorder or condition in a mammalian subject in need thereof, comprising administering to said subject at least once an effective amount of the ABP of any one of items 1 to 43, the NAC of item 45, cells of item 46 or item 47, or the pharmaceutical composition of item 48 or 49.

Item 54. The ABP, NAC, cells, or pharmaceutical composition for the use according to item 52, or the method of item 53, wherein an effective amount is administered at least once to said subject of: (i) said ABP which is between about 0.1 mg/kg and about 10 mg/kg per administration; or (ii) said NAC which is between about 0.05 μg/kg and about 500 μg/kg per administration; or (iii) said cells which is between about 10 cells/kg and about 1×10\hat{ }7 cells/kg.

Item 55. The ABP, NAC, cells, or pharmaceutical composition for the use according to item 52 or 54, or the method of item 53 or 54, wherein said disease, disorder or condition is a proliferative disorder, for example a tumour or cancer, and/or an infection and/or an immune disorder, for example an immune deficiency.

Item 56. The ABP, NAC, cells, or pharmaceutical composition for the use according to item 52, 54 or 55, or the method of any one of items 53 to 55, wherein said disease, disorder or condition is characterised by (i) aberrant expression and/or activity of a human OR10H1, or a paralogue, orthologue or other variant thereof, or (ii) an aberrant immune response.

Item 57. The ABP, NAC, cells, or pharmaceutical composition for the use according to any one of items 52 and 54 to 56 or the method of any one of items 53 to 56, wherein said disease, disorder or condition is associated with a pathological CTL response.

Item 58. The ABP, NAC, cells, or pharmaceutical composition for the use according to any one of items 52 and 54 to 57 or the method of any one of items 53 to 57, wherein said ABP is an inhibitor or antagonist of expression, function, activity and/or stability of said OR10H1 or variant; preferably is an inhibitor or antagonist of the function and/or activity of said OR10H1 or variant.

Item 59. The ABP, NAC, cells, or pharmaceutical composition for the use according to any one of items 52 and 54 to 58 or the method of any one of items 53 to 58, wherein: (i) a cell-mediated immune response, such as that mediated by an activated CTL, is enhanced in said subject; and/or (ii) immune cell, such as T-cell, activity and/or survival is increased in said subject.

Item 60. The ABP, NAC, cells, or pharmaceutical composition for the use or the method of item 59, wherein type-I cytokine secretion by the immune cells mediating said cell-mediated immune response, such as by CTLs, is increased in said subject, such as one or more cytokines independently selected from the list consisting of: IFN-gamma, IL-2 and TNF-alpha.

Item 61. The ABP, NAC, cells, or pharmaceutical composition for the use or the method of item 59 or 60, wherein: (i) said enhancement of a cell-mediated immune response; and/or (ii) said increase in immune cell activity and/or survival, is associated with: (x) a reduced phosphorylation of CREB in the immune cells mediating said cell-mediated immune response, or having said activity; and/or (y) activation of lymphocyte-specific protein tyrosine kinase (Lck), for example by reduced phosphorylation of the LcK Tyr505 domain in said immune cells.

Item 62. The ABP, NAC, cells, or pharmaceutical composition for the use according to any one of items 52 and 54 to 61 or the method of any one of items 53 to 61, wherein said disease, disorder or condition is a cancer expressing said OR10H1 or variant; for example an OR10H1-positive cancer.

Item 63. The ABP, NAC, cells, or pharmaceutical composition for the use according to any one of items 52 and 54 to 62, or the method of any one of items 53 to 62, wherein said disease, disorder or condition is selected from the list consisting of: melanoma, pancreatic cancer and colorectal cancer.

Item 64. The ABP, NAC, cells, or pharmaceutical composition for the use according to any one of items 52 and 54 to 63, or the method of any one of items 53 to 63, wherein said treatment comprises aiding a cell-mediated immune response, such as a T-cell mediated immune response, in said subject.

Item 65. The ABP, NAC, cells, or pharmaceutical composition for the use according to any one of items 52 and 54 to 64, or the method of any one of items 53 to 64, wherein said treatment comprises a transfer of cells to said subject, preferably a transfer of immune cells to the subject, for example adoptive T-cell transfer.

Item 66. A method of detecting a human OR10H1 or a paralogue, orthologue or other variant thereof in a sample, comprising contacting the sample with an ABP of any one of items 1 to 43 and detecting binding between said ABP and said OR10H1, paralogue, orthologue or other variant.

Item 67. A method of determining whether a mammalian subject, such as a human, has or is at risk of developing a phenotype associated with cellular resistance against a cell-mediated immune response, said method comprising

(a) the steps of:

(i) providing a biological sample from said subject, said sample comprising cells or tissue of said subject, or an extract of said cells or tissue; and

(ii) practicing the method of item 66 with said sample;

or

(b) practicing the method of item 66 on a biological sample from said subject, said sample comprising cells or tissue of said subject, or an extract of said cells or tissue;

wherein the detection of a human OR10H1 or paralogue, orthologue or other variant thereof in said sample indicates a phenotype associated with cellular resistance against the cell-mediated immune response in said subject.

Item 68. The method of item 67, wherein said biological sample is a tissue sample from said subject, such as a sample of a tumour or a cancer from said subject.

Item 69. The method of item 67 or 68, wherein a detected amount of said OR10H1 or variant greater than a standard or cut-off value indicates a phenotype associated with cellular resistance against the cell-mediated immune response in said subject.

Item 70. The ABP of any one of items 1 to 43, for use in diagnosis, such as in the detection of a disease, disorder or condition in a mammalian subject, such as a human patient.

Item 71. A kit for detecting human OR10H1 or a paralogue, orthologue or other variant thereof in a biological sample comprising:

(i) at least one ABP of any one of items 1 to 43 and

(ii) optionally, instructions for, and/or one or more additional components suitable for, detecting the binding between said ABP and said OR10H1 or variant.

Item 72. A cell, which is either: (i) a hybridoma capable of expressing an ABP of any one of items 1 to 43; or (ii) a cell comprising at least one NAC of item 45 encoding an ABP or a component of an ABP according to any one of items 1 to 43.

Item 73. A method of producing an ABP of any one of items 1 to 43, comprising culturing one or more cells of item 72 under conditions allowing the expression of said ABP.

Item 74. The method of item 73, further comprising a step of isolating said ABP.

Item 75. A method of manufacturing a pharmaceutical composition of item 48 or 49, comprising formulating the ABP isolated by the method of 74 into a pharmaceutically acceptable form.

Item 76. The method of item 75, further comprising a step of combining said ABP with a pharmaceutically acceptable excipient or carrier.

Sequences

SEQ ID NO: 1 (human OR10H1 wild-type (WT) full length amino acid sequence):
MQRANHSTVTQFILVGFSVFPHLQLMLFLLFLLMYLFTLLGNLLIMATVWSERSLHTPMYLFLCALSV
SEILYTVAIIPRMLADLLSTQRSIAFLACASQMFFSFSFGFTHSFLLTVMGYDRYVAICHPLRYNVLM
SPRGCACLVGCSWAGGLVMGMVVTSAIFHLAFCGHKEIHHFACHVPPLLKLACGDDVLVVAKGVGLVC
ITALLGCFLLILLSYAFIVAAILKIPSAEGRNKAFSTCASHLTVVVVHYGFASVIYLKPKSPQSLEGD
TLMGITYTVLTPFLSPIIFSLRNKELKVAMKKTFFSKLYPEKNVMM
SEQ ID NO: 2 (human OR10H1 G16R full length amino acid sequence):
MQRANHSTVTQFILVRFSVFPHLQLMLFLLFLLMYLFTLLGNLLIMATVWSERSLHTPMYLFLCALSV
SEILYTVAIIPRMLADLLSTQRSIAFLACASQMFFSFSFGFTHSFLLTVMGYDRYVAICHPLRYNVLM
SPRGCACLVGCSWAGGLVMGMVVTSAIFHLAFCGHKEIHHFACHVPPLLKLACGDDVLVVAKGVGLVC
ITALLGCFLLILLSYAFIVAAILKIPSAEGRNKAFSTCASHLTVVVVHYGFASVIYLKPKSPQSLEGD
TLMGITYTVLTPFLSPIIFSLRNKELKVAMKKTFFSKLYPEKNVMM
SEQ ID NO: 3 (human OR10H1 A65V/G16R full length amino acid sequence):
MQRANHSTVTQFILVRFSVFPHLQLMLFLLFLLMYLFTLLGNLLIMATVWSERSLHTPMYLFLCVLSV
SEILYTVAIIPRMLADLLSTQRSIAFLACASQMFFSFSFGFTHSFLLTVMGYDRYVAICHPLRYNVLM
SPRGCACLVGCSWAGGLVMGMVVTSAIFHLAFCGHKEIHHFACHVPPLLKLACGDDVLVVAKGVGLVC
ITALLGCFLLILLSYAFIVAAILKIPSAEGRNKAFSTCASHLTVVVVHYGFASVIYLKPKSPQSLEGD
TLMGITYTVLTPFLSPIIFSLRNKELKVAMKKTFFSKLYPEKNVMM
SEQ ID NO: 4 (human OR10H1 H175Q full length amino acid sequence):
MQRANHSTVTQFILVGFSVFPHLQLMLFLLFLLMYLFTLLGNLLIMATVWSERSLHTPMYLFLCALSV
SEILYTVAIIPRMLADLLSTQRSIAFLACASQMFFSFSFGFTHSFLLTVMGYDRYVAICHPLRYNVLM
SPRGCACLVGCSWAGGLVMGMVVTSAIFHLAFCGHKEIQHFACHVPPLLKLACGDDVLVVAKGVGLVC
ITALLGCFLLILLSYAFIVAAILKIPSAEGRNKAFSTCASHLTVVVVHYGFASVIYLKPKSPQSLEGD
TLMGITYTVLTPFLSPIIFSLRNKELKVAMKKTFFSKLYPEKNVMM
SEQ ID NO: 5 (human OR10H1 WT EC domain 1 amino acid sequence):
MQRANHSTVTQFILVGFSVFPHLQL
SEQ ID NO: 6 (human OR10H1 WT EC domain 2 amino acid sequence):
AIIPRMLADLLSTQRSIAFLACAS
SEQ ID NO: 7 (human OR10H1 WT EC domain 3 amino acid sequence):
SAIFHLAFCGHKEIHHFACHVPPLLKLACGDDVLVVA
SEQ ID NO: 8 (human OR10H1 WT EC domain 4 amino acid sequence):
YLKPKSPQSLEGD
SEQ ID NO: 9 (human OR10H1 G16R EC domain 1 amino acid sequence):
MQRANHSTVTQFILVRFSVFPHLQL
SEQ ID NO: 10 (human OR10H1 H175Q EC domain 3 amino acid sequence):
SAIFHLAFCGHKEIQHFACHVPPLLKLACGDDVLVVA
SEQ ID NO: 11 (Cynomolgus orthologue full length amino acid sequence):
MQRANHSTVTQFILVGFSAFPDLQLMLFLLFLLMYLFTLLGNLLIMATVWSERSLHTPMYLFLCALSV
SEILYTVAIIPRMLADLLSTQRSIAFLACASQMFFSFSFGFTHSFLLTVMGYDRYVAICRPLRYNVLM
SPRGGACLVGCSWAGGSVMGLVVTTAIFHLTFCGPSEIHHFACHVPPLLKLACGNNVPAVALGVGLVC
ITALLGCFLLILLSYAFIVAAILKIPSAEGRNKAFSTCASHVTVVVVHYGFASVIYLKPKGPQSLEGD
TLMGITYTVLTPFLSPIIFSLRNKELKVAMKTFFSKLYPEKNVTI
SEQ ID NO: 12 (human OR10H1 A167T full length amino acid sequence):
MQRANHSTVTQFILVGFSVFPHLQLMLFLLFLLMYLFTLLGNLLIMATVWSERSLHTPMYLFLCALSV
SEILYTVAIIPRMLADLLSTQRSIAFLACASQMFFSFSFGFTHSFLLTVMGYDRYVAICHPLRYNVLM
SPRGCACLVGCSWAGGLVMGMVVTSAIFHLTFCGHKEIHHFACHVPPLLKLACGDDVLVVAKGVGLVC
ITALLGCFLLILLSYAFIVAAILKIPSAEGRNKAFSTCASHLTVVVVHYGFASVIYLKPKSPQSLEGD
TLMGITYTVLTPFLSPIIFSLRNKELKVAMKKTFFSKLYPEKNVMM
SEQ ID NO: 13 (human OR10H1 A167T EC domain 3 amino acid sequence):
SAIFHLTFCGHKEIHHFACHVPPLLKLACGDDVLVVA
SEQ ID NO: 14 (human OR10H5 wild-type (WT) full length amino acid sequence):
MQGLNHTSVSEFILVGFSAFPHLQLMLFLLFLLMYLFTLLGNLLIMATVWSERSLHMPMYLFLCALSI
TEILYTVAIIPRMLADLLSTQRSIAFLACASQMFFSFSFGFTHSFLLTVMGYDRYVAICHPLRYNVLM
SLRGCTCRVGCSWAGGLVMGMVVTSAIFHLAFCGHKEIHHFFCHVPPLLKLACGDDVLVVAKGVGLVC
ITALLGCFLLILLSYAFIVAAILKIPSAEGRNKAFSTCASHLTVVVVHYGFASVIYLKPKGPQSPEGD
TLMGITYTVLTPFLSPIIFSLRNKELKVAMKKTCFTKLFPQNC
SEQ ID NO: 15 (human OR10H2 wild-type (WT) full length amino acid sequence):
MLGLNHTSMSEFILVGFSAFPHLQLMLFLLFLLMYLFTLLGNLLIMATVWSERSLHTPMYLFLCVLSV
SEILYTVAIIPRMLADLLSTQRSIAFLACASQMFFSFSFGFTHSFLLTVMGYDRYVAICHPLRYNVLM
SPRGCACLVGCSWAGGSVMGMVVTSAIFQLTFCGSHEIQHFLCHVPPLLKLACGNNVPAVALGVGLVC
IMALLGCFLLILLSYAFIVADILKIPSAEGRNKAFSTCASHLIVVIVHYGFASVIYLKPKGPHSQEGD
TLMATTYAVLTPFLSPIIFSLRNKELKVAMKRTFLSTLYSSGT
SEQ ID NO: 16 (human OR10H5 WT EC domain 1 amino acid sequence):
MQGLNHTSVSEFILVGFSAFPHLQL
SEQ ID NO: 17 (human OR10H2 WT EC domain 1 amino acid sequence):
MLGLNHTSMSEFILVGFSAFPHLQL
SEQ ID NO: 18 (human OR10H2 WT EC domain 2 amino acid sequence):
AIIPRMLADLLSTQRSIAFLACAS
SEQ ID NO: 19 (human OR10H5 WT EC domain 2 amino acid sequence):
AIIPRMLADLLSTQRSIAFLACAS
SEQ ID NO: 20 (human OR10H5 WT EC domain 3 amino acid sequence):
SAIFHLAFCGHKEIHHFFCHVPPLLKLACGDDVLVVA
SEQ ID NO: 21 (human OR10H2 WT EC domain 3 amino acid sequence):
SAIFQLTFCGSHEIQHFLCHVPPLLKLACGNNVPAVA
SEQ ID NO: 22 (human OR10H5 WT EC domain 4 amino acid sequence):
YLKPKGPQSPEGD
SEQ ID NO: 23 (human OR10H2 WT EC domain 4 amino acid sequence):
YLKPKGPHSQEGD
SEQ ID NOs: 24 to 27 (Exemplary siRNAs against ORH10H)-see Table E1
SEQ ID NOs: 28 to 32 (Exemplary shRNAs against ORH10H)-see Table E2
SEQ ID NO: 33 (1C3-A1-A1 heavy-chain CDR1 amino acid sequence)
FTFDNAW
SEQ ID NO: 34 (1C3-A1-A1 heavy-chain CDR2 amino acid sequence)
IKAKSNNYAT
SEQ ID NO: 35 (1C3-A1-A1 heavy-chain CDR3 amino acid sequence)
TRLYYD
SEQ ID NO: 36 (1C3-A1-A1 heavy-chain variable domain amino acid sequence)
EVQLVETGGNLVQPGKSLKLTCATSGFTFDNAWMHWVRQSPEKQLEWVAQIKAKSNNYATYYAESVKG
RFTISRDDSKSSVYLQMNRLKEEDTAIYYCTRLYYDWGQGVMVTVSS
SEQ ID NO: 37 (1C3-A1-A1 light-chain CDR1 amino acid sequence)
QDIGNY
SEQ ID NO: 38 (1C3-A1-A1 light-chain CDR2 amino acid sequence)
RAT
SEQ ID NO: 39 (1C3-A1-A1 light-chain CDR3 amino acid sequence)
LQHKQYPL
SEQ ID NO: 40 (1C3-A1-A1 light-chain variable domain amino acid sequence)
DIQMTQSPSSISVSLGDRFTFTCRASQDIGNYLSWFQQKPEKSPKLMIYRATNLEDGVPSRFSGSRSG
SDYSLTINSLESEDTGIYFCLQHKQYPLTFGSGTKLEIKR
SEQ ID NO: 41 (1C3-A1-A2 heavy-chain CDR1 amino acid sequence)
FTFSNYD
SEQ ID NO: 42 (1C3-A1-A2 heavy-chain CDR2 amino acid sequence)
ISHSGDSTYF
SEQ ID NO: 43 (1C3-A1-A2 heavy-chain CDR3 amino acid sequence)
HGVYTN
SEQ ID NO: 44 (1C3-A1-A2 heavy-chain variable domain amino acid sequence)
EVQLQESGGGLIQPGRSLKLSCAASGFTFSNYDMAWVRQAPTKGLEWVASISHSGDSTYFRASVKGRF
TVSRDNAKSSLYLQMDSLRSEDTATYYCARHGVYTNYGIWFAYWGQGTLVTVSS
SEQ ID NO: 45 (1C3-A1-A2 light-chain CDR1 amino acid sequence)
QDIGNY
SEQ ID NO: 46 (1C3-A1-A2 light-chain CDR2 amino acid sequence)
RAT
SEQ ID NO: 47 (1C3-A1-A2 light-chain CDR3 amino acid sequence)
LQHKQYPL
SEQ ID NO: 48 (1C3-A1-A2 light-chain variable domain amino acid sequence)
DIQMTQSPSSISVSLGDRFTFTCRASQDIGNYLSWFQQKPEKSPKLMIYRATNLEDGVPSRFSGSRSG
SDYSLTINSLESEDTGIYFCLQHKQYPLTFGSGTKLEIKR
SEQ ID NO: 49 (8A11-B9-A1 heavy-chain CDR1 amino acid sequence)
FTFSSAW
SEQ ID NO: 50 (8A11-B9-A1 heavy-chain CDR2 amino acid sequence)
IKGKSNNYAT
SEQ ID NO: 51 (8A11-B9-A1 heavy-chain CDR3 amino acid sequence)
TWFGPMDA
SEQ ID NO: 52 (8A11-B9-A1 heavy-chain variable domain amino acid sequence)
EVQLQESGGRLVQPGKSLKLTCAASGFTFSSAWIHWVRQSPEKQLEWVAQIKGKSNNYATYYAESVKG
RFTISRDDSKSSVYLQMNSLKEEDTAIYYCTWFGPMDAWGQGASVTVSS
SEQ ID NO: 53 (8A11-B9-A1 light-chain CDR1 amino acid sequence)
KSLLHNNGKTF
SEQ ID NO: 54 (8A11-B9-A1 light-chain CDR2 amino acid sequence)
WMS
SEQ ID NO: 55 (8A11-B9-A1 light-chain CDR3 amino acid sequence)
QQFLEYPL
SEQ ID NO: 56 (8A11-B9-A1 light-chain variable domain amino acid sequence)
DIVMTQGALPNPVPSGESASITCQSSKSLLHNNGKTFLNWYLQRPGQSPQLLIYWMSTRASGVSDRFS
GSGSGADFTLKISSVEAEDVGVYYCQQFLEYPLTFGSGTKLEIKR

### EXAMPLES

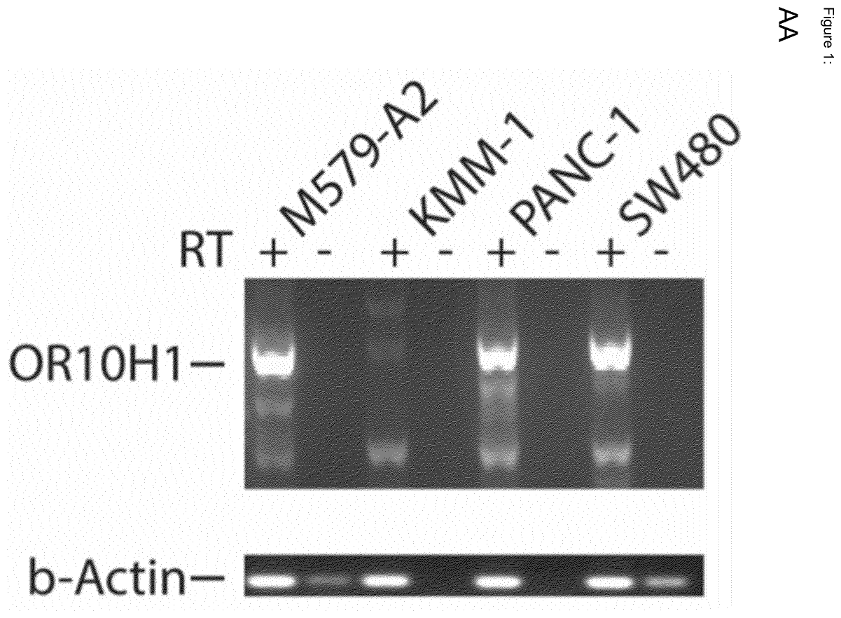

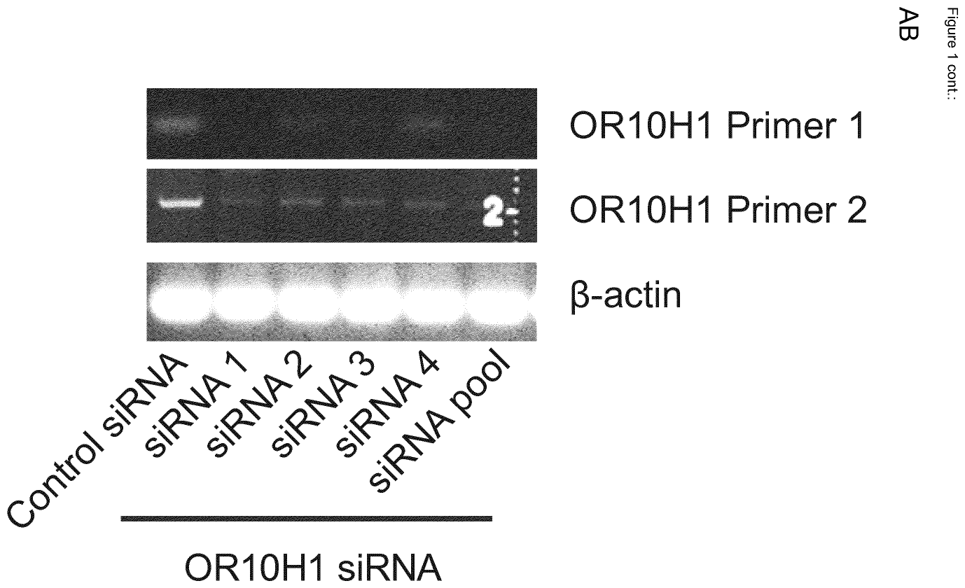

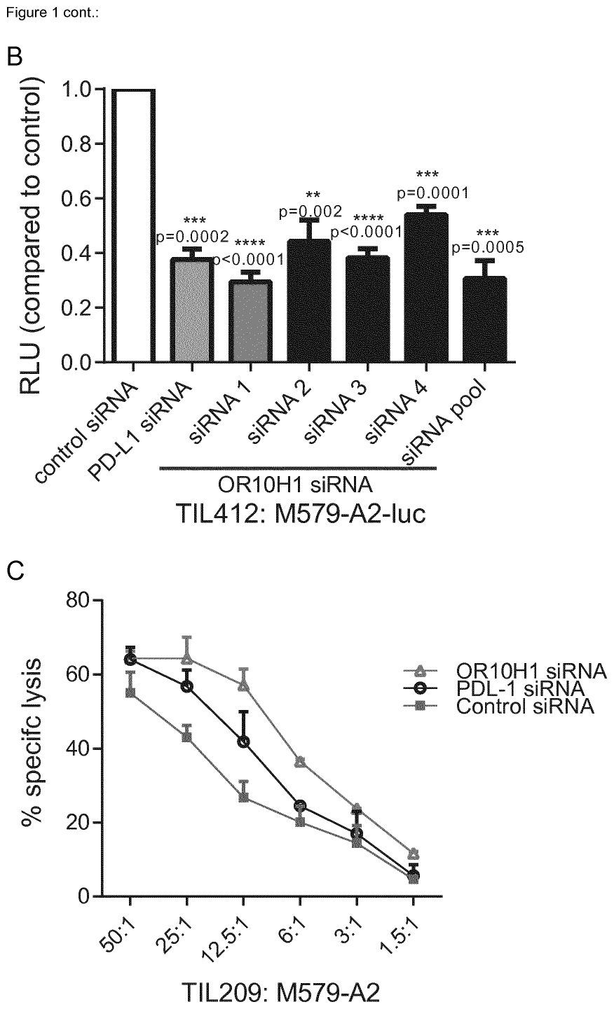

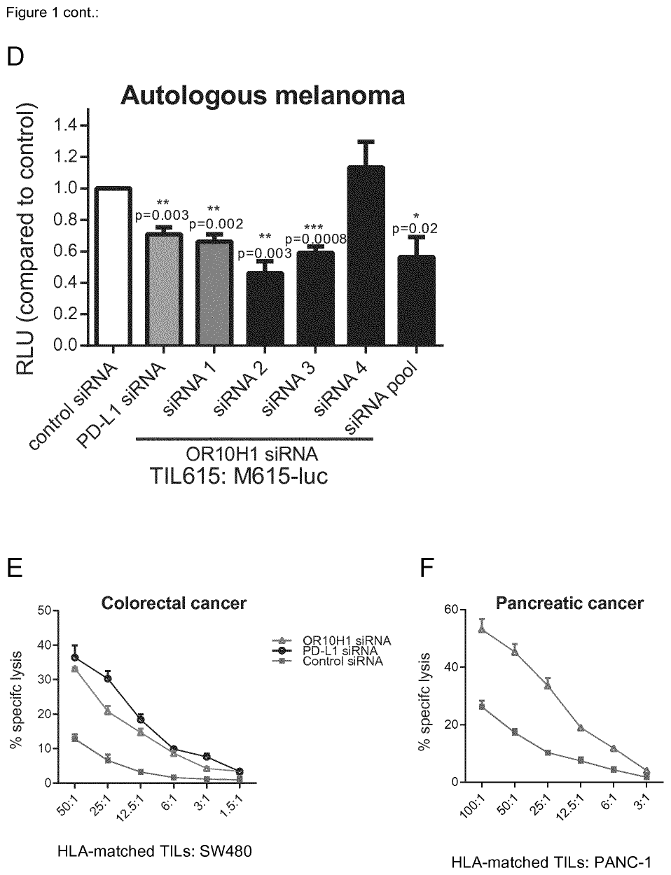

### Example 1: OR10H1 Knock-Down Increases TIL-Mediated Killing of Solid tumors (FIG. 1)

RT-PCR data and expression database searches suggest that the cell surface-expressed gene OR10H1 is expressed by melanoma, PDAC and colorectal cancer. Therefore, two OR10H1-specific PCR primers are tested by sequencing the respective RT-PCR amplicons according to standard procedures. The results show (FIG. 1AA) that OR10H1 is expressed by the following cells: M579-A2 (melanoma) (Machlenkin et al, 2008; Cancer Res 68:2006-13), PANC-1 (PDAC) and SW480 (colorectal) (both, ATCC), but not expressed by KMM-1 (myeloma) (Namba et al, 1989; In Vitro Cell Dev Biol 8:723-9).

SMART pool siRNA against OR10H1 (GE Dharmacon) were tested (by RT-PCR) for their activity in downregulating OR10H1 mRNA in M579 (melanoma) cells. Following transfection into M579-A2 cells, all siRNAs show a knock-down of OR10H1 (FIG. 1AB). siRNA 1 shows a complete absence of OR10H1 mRNA, siRNA 3 and pooled OR10H1 siRNAs show a strong reduction, siRNA 2 and 4 show a weaker but clear reduction in OR10H1 transcription. Details of the respective siRNAs are set out in Table E1.

TABLE E1
Exemplary siRNAs against ORH10H1

| | | GE Dharmacon | SEQ ID |
|---|---|---|---|
| Name | Sequence | order# | No. |
| OR10H1 siRNA 1 | GGAGACACCUUGAUGGGCA | D-020479-01 | 24 |
| OR10H1 siRNA 2 | AGUAAACUCUACCCAGAAA | D-020479-02 | 25 |
| OR10H1 siRNA 3 | GCAGAGAGCCAAUCACUCC | D-020479-03 | 26 |
| OR10H1 siRNA 4 | GGUCGUGCACUAUGGCUUU | D-020479-04 | 27 |
| OR10H1 pool | | M-020479-01 | |

It was surprising to find that all siRNAs show an impact on TIL412-mediated killing of M579-A2-luc at 5:1 Effector to Target cell (E:T) ratio (FIG. 1B). Transfection of M579-A2-luc cells with OR10H1 siRNA 1 increased HLA-matched patient-derived tumor-infiltrating lymphocyte (TIL, clone 412)-mediated killing to 70% (30% remaining melanoma cells compared to a scrambled negative control siRNA; p=<0.0001). This increase in TIL-mediated lysis is stronger than for PD-L1 positive control knock-down (39% remaining cells). Overall, the effect on the phenotype is comparable to the knock-down efficacy on OR10H1 mRNA. Only siRNA 3 shows an effect on M579-A2-luc viability (without the presence of TILs; data not shown).

TIL-mediated lysis of M579-A2 was validated in chromium release assay (4 h co-culture). Another patient-derived and HLA-matched TIL culture (TIL209) was used to show that the knock-down effect on TIL-mediated killing does not depend specifically on TIL412. Knock-down of OR10H1 (siRNA 1) strongly increased the lysis of M579-A2-luc in all effector to target (E:T) ratios compared to the negative control siRNA (57% to 27% specific lysis at 12.5 E:T ratio; FIG. 1C). Indeed, this effect of OR10H1 knock-down was stronger than the PD-L1 positive control knock-down (41% specific lysis at 12.5 E:T ratio; FIG. 1C).

OR10H1 knock-down increased TIL-mediated killing in an autologous melanoma setting. For this, M615 tumor cells and TIL615 were derived from the same patient, and M615 was stably transfected with luciferase to produce M615-luc. The basic killing of M615-luc by TIL615 was very low. However, knock-down of OR10H1 increased TIL-mediated killing compared to the negative control siRNA (FIG. 1D).

Furthermore, it was tested whether OR10H1 knock-down also has an effect in other cancers. Surprisingly, OR10H1 knock-down increased the TIL-mediated tumor cell killing in colorectal cancer (CRC) and pancreatic cancer cells. SW480 (ATCC) and PANC-1 cells were analyzed for their expression of OR10H1 (see above) and the knock-down efficacy of the siRNAs were validated. The CRC cell line SW480 and the PANC-1 cell line were each co-cultured with corresponding HLA-matched TILs for chromium release (4 h co-culture). OR10H1 siRNA 1 almost tripled the lysis of SW480 by TILs in all effector to target ratios compared to the negative control siRNA (33% to 12.8% specific lysis at 50:1 E:T ratio; FIG. 1E). Indeed, knock-down of OR10H1 was almost as effective as knock-down of PD-L1 positive control on TIL-mediated lysis (FIG. 1E). Such effect was also surprising to see in pancreatic cancer cells: knock-down of OR10H1 (siRNA 1) doubled the lysis of PANC-1 by TILs in all effector to target ratios compared to the negative control siRNA (53% to 26% specific lysis at 50:1 E:T ratio; FIG. 1F).

The effects of siRNA knock-down on TIL-mediated killing and lysis in a chromium release assay were investigated as described in Khandelwal et al, 2015 (EMBO Mol Med 7:450-63). Tumor-infiltrating lymphocytes 412 and 209 microcultures were expanded from an inguinal lymph node of a melanoma patient as described in Dudley et al, 2010 (Clin Cancer Res 16:6122-31). M615, TIL615 and other patient-derived TIL cells were obtained analogously as described in Dudley et al, 2010. M579-A2-luc cells and M615-luc cells were produced from M579-A2 and M615, respectively (Machlenkin et al, 2008; Cancer Res 68:2006-13).

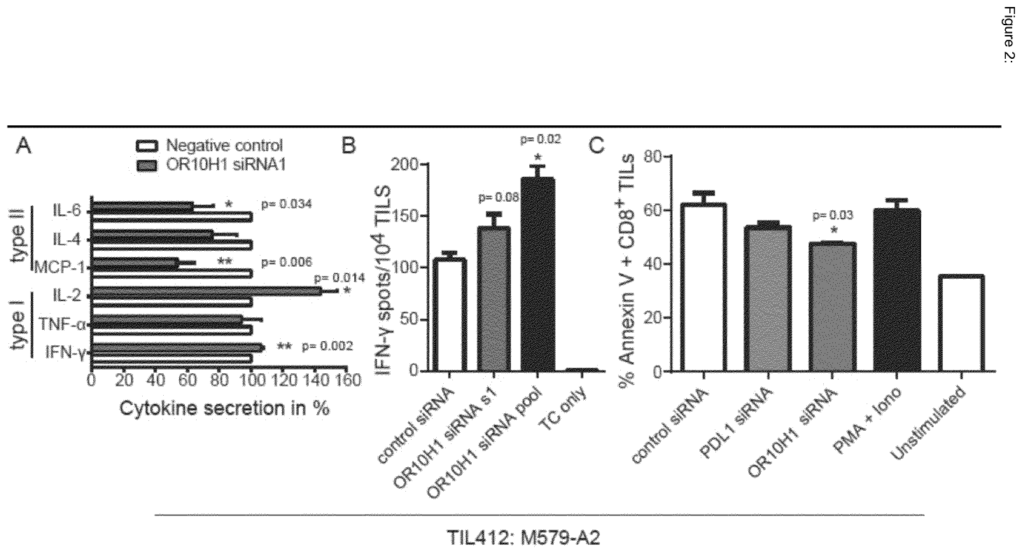

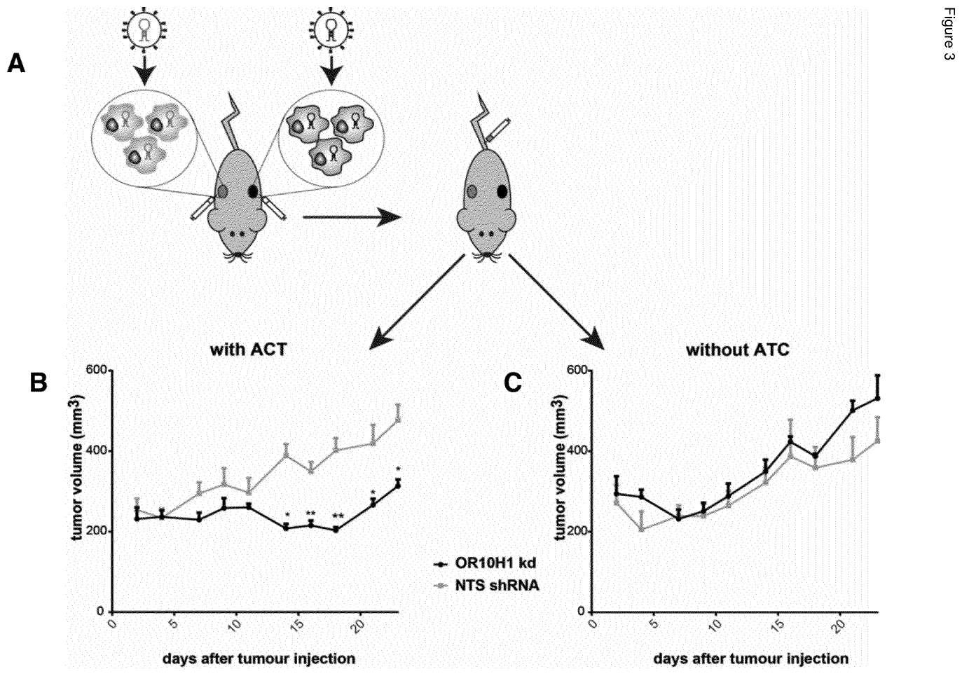

### Example 2: OR10H1 Knock-Down Increases TIL Activity and Survival (FIG. 2)

A co-culture of M579-A2 and TIL412 leads to a switch in cytokines secreted by TILs (from immune-suppressive type II to immune-activating type I), showing an increase in anti-tumor response. Cells were co-cultured for 20 hours and the cytokine concentrations in the supernatant were measured by Luminex as described in Khandelwal et al, 2015. As controls unstimulated TILs (no tumor cells) and over-stimulated TILs (PMA and ionomycin) were used (not shown).

As expected, unstimulated TILs did not secrete considerable amounts of cytokines, whereas over-stimulated TILs secreted dramatically increased amounts. However, the inventors were surprised to observe that knock-down of OR10H1 on melanoma increased type I cytokine secretion by TIL412 (FIG. 2A). IFN-gamma secretion increased significantly from 2825 to 3037 pg/ml (increase of 8%; p=0.0236) compared to the negative control. IL-2 secretion significantly increased from 1079 to 1701 pg/ml (increase of 58%; p=0.045). Furthermore, knock-down of OR10H1 was found to surprisingly decrease type II cytokine secretion by TIL412 (FIG. 2A). MCP-1 secretion significantly decreased from 6658 to 4035 pg/ml (decrease of 39%; p=0.016). IL-6 secretion significantly decreased from 49.4 to 31 pg/ml (decrease of 37%; p=0.028). IL-4 secretion decreased from 14.75 to 11.8 pg/ml (20%; trend).

Knock-down of OR10H1 increased the number of IFN-gamma producing TILs. Melanoma cells and TILs were co-cultured for 20 h and the number of IFN-gamma producing cells was measured using ELISpot as described in Khandelwal et al, 2015. Although TILs did not produce IFN-gamma without interaction with melanoma cells, knock-down of OR10H1 (pooled siRNA) significantly increased the number of IFN-gamma spots/104 TILs from 107.75 to 187.75 (increase of 73%; p=0.02; FIG. 2B).

Without being bound by theory, the effect of OR10H1 knock-down (in melanoma cells) on TIL activity appeared to depend on the HLA-TCR interaction (not shown). The inventors then compared the amounts of IFN-gamma after the co-culture (6 h) of M579-A2 melanoma cells with TILs. If melanoma cells did not express HLA-A2 (M579), IFN-gamma secretion was abrogated regardless of the knock-down of OR10H1. However, knock-down of OR10H1 on melanoma cells surprisingly reduced the induction of apoptosis in TILs after co-culture suggesting that OR10H1 knock-down prolonged survival and/or increased proliferation of TILs. Melanoma cells and TILs were co-cultured for 6 h and the percentage of Annexin V-positive CD8+ TILs (FACS staining) was calculated, in each case according to standard procedures. TILs which were not co-cultured or activated showed 35% of Annexin V-positive CD8+ T cells. Over-activation with PMA and ionomycin resulted in 60% apoptotic CD8+ TCs. Interestingly, co-culture with OR10H1-positive M579-A2 (scrambled siRNA control) increased the percentage of apoptotic CD8+ TCs to 62%. Surprisingly, knock-down of OR10H1 prior to co-culture reduced the number of apoptotic CD8+ TILs to 47% (FIG. 2C). The induction of apoptosis (compared to unstimulated TILs) is half as large if OR10H1 is not expressed in the melanoma cells (p=0.032). Knock-down of PD-L1 decreased the percentage of apoptotic CD8+ TCs to 53% (non-significant compared to scrambled siRNA control).

### Example 3: OR10H1 Functions as an Immune Checkpoint In Vivo (FIG. 3)

In order to assess OR10H1 function in vivo, a stable OR10H1 knock-down M579-A2 line was generated using lentiviral shRNA particles (MISSION shRNA for OR10H1; Sigma Aldrich), analogously as described in Khandelwal et al, 2015 for the stable CCR9 knockdown in M579-A2. The stable knock-down of OR10H1 increased the specific lysis of M579-A2 by TIL412 and TIL209 in vitro (chromium release) compared to negative target sequence (NTS) control-transduced M579-A2 (not shown). ShRNA 4 showed the strongest impact on OR10H1 expression and functionality, whereas stable knock-down of OR10H1 did not affect viability or proliferation of M579-A2 in vitro (not shown). Details of the respective shRNAs are set out in Table E2.

TABLE E2
Exemplary shRNAs against ORH10H1

| | | Sigma Aldrich | SEQ ID |
|---|---|---|---|
| Name | Sequence | order# | No. |
| OR10H1 shRNA 1 | GTTCCTGCTGATGTACCTGTT | TRCN0000011786 | 28 |
| OR10H1 shRNA 2 | TGCGCTACAACGTGCTCATGA | TRCN0000357706 | 29 |
| OR10H1 shRNA 3 | TGGCTTTGCCTCCGTCATTTA | TRCN0000357707 | 30 |
| OR10H1 shRNA 4 | TCTGCTGAAGGTCGGAACAAG | TRCN0000357708 | 31 |
| OR10H1 shRNA 5 | ACACAAGGAGATCCACCATTT | TRCN0000357775 | 32 |

OR10H1 knock-down and NTS control—transduced M579-A2 cells (in matrigel) were injected subcutaneously into the flanks of immunodeficient (NOD scid gamma; NSG) mice (FIG. 3A) and on day 2 and 9 after tumor inoculation, TIL412 cells were injected intravenously (adoptive cell transfer; ACT). Subsequently the tumor size was measured for 24 days. A control group of mice was injected with tumor cells but did not receive ACT, so as to validate the effect of OR10H1 knock-down on tumor growth without the presence of TILs.

Tumor growth is strongly reduced in OR10H1 knock-down (kd) tumors (i.e., OR10H1-negative tumors) after ACT with TIL412 but not without the ACT (FIGS. 3B and C). On day 7 (273 mm³) the tumors start to grow regardless of the presence or absence of OR10H1. There is no significant difference in tumor growth of OR10H1 knock-down M579-A2 compared to that of the NTS shRNA control group (FIG. 3C). In contrast, and to the surprise of the inventors, if the mice were i.v. injected with TIL412 as ACT, M579-A2 tumor growth of OR10H1-negative (kd), but not growth of shRNA control M579-A2 tumors (ie, OR10H1-negative), was significantly reduced after day 11 (FIG. 3B). OR10H1 knock down tumors significantly reduced in volume from 260 to 207 mm³ between day 11 and 14 after inoculation. In contrast, and over the same period, the NTS control tumors grew substantially from 296 to 389 mm³ (p=0.013). On day 18 the average OR10H1-negative tumor (kd) had a volume of only 203 mm³ compared to 401 mm³ for the average size of NTS control (ie OR10H1-positive) tumors (p=0.004). Although after day 18, the OR10H1-negative tumors do start to grow again, their tumor mass remains significantly reduced compared to NTS control (i.e. OR10H1-positive) tumors.

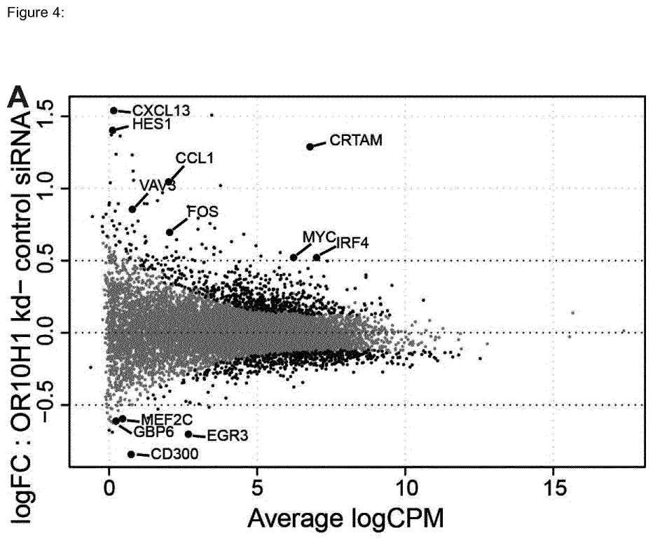

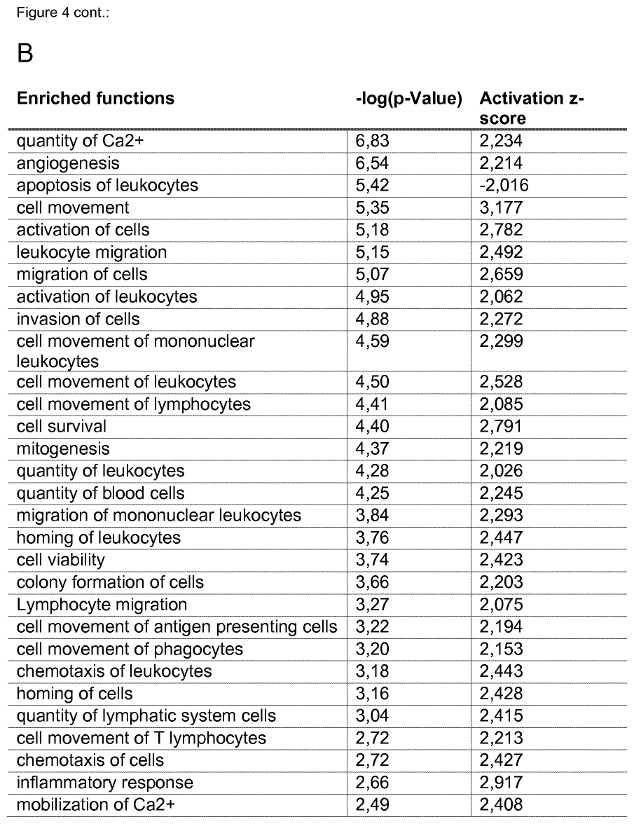

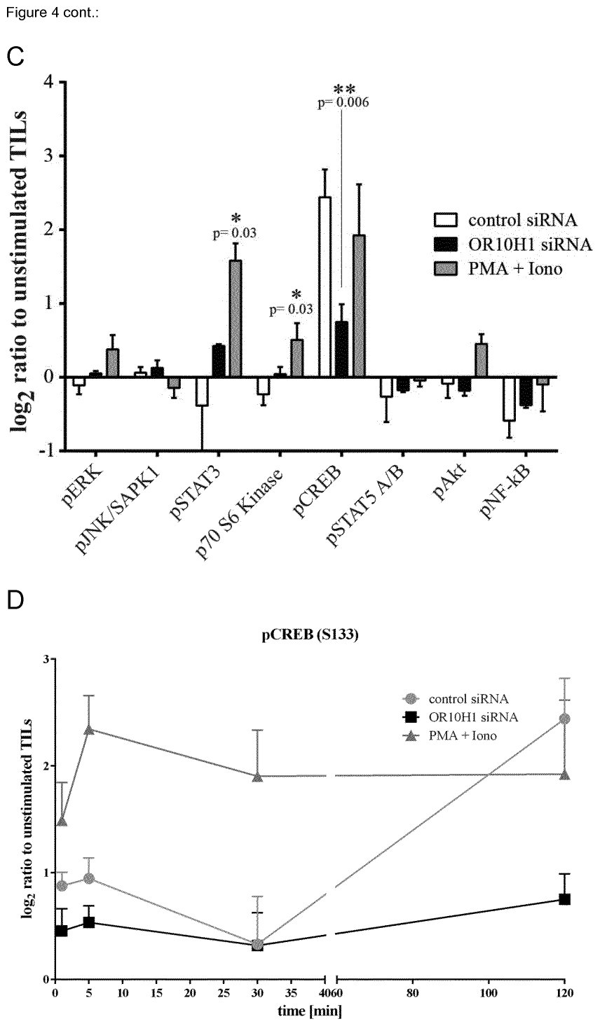

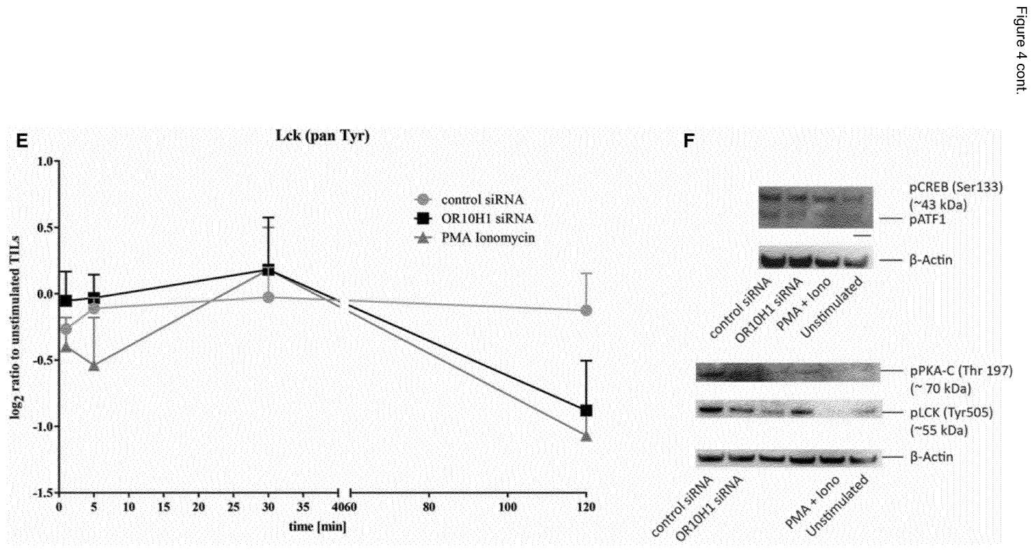

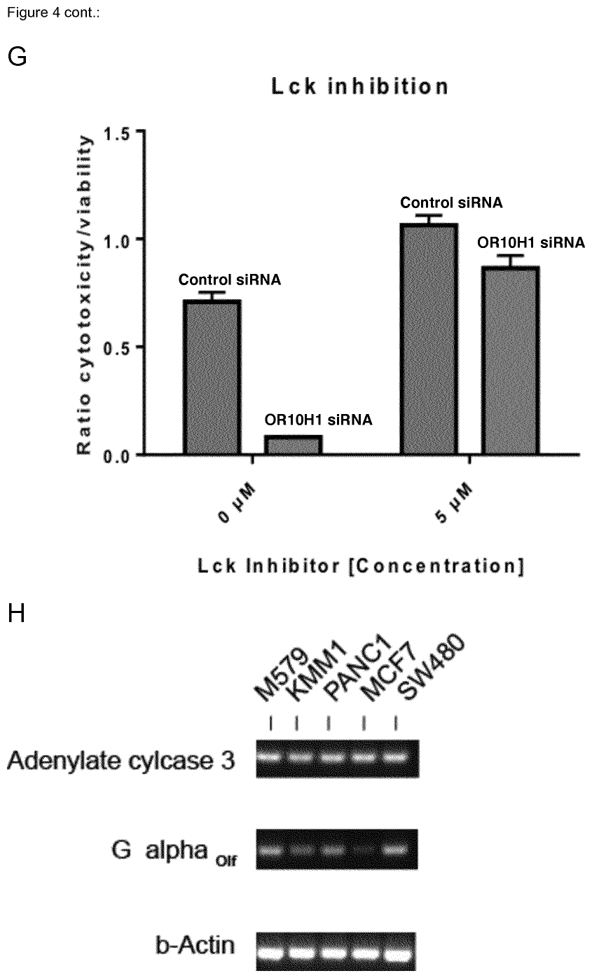

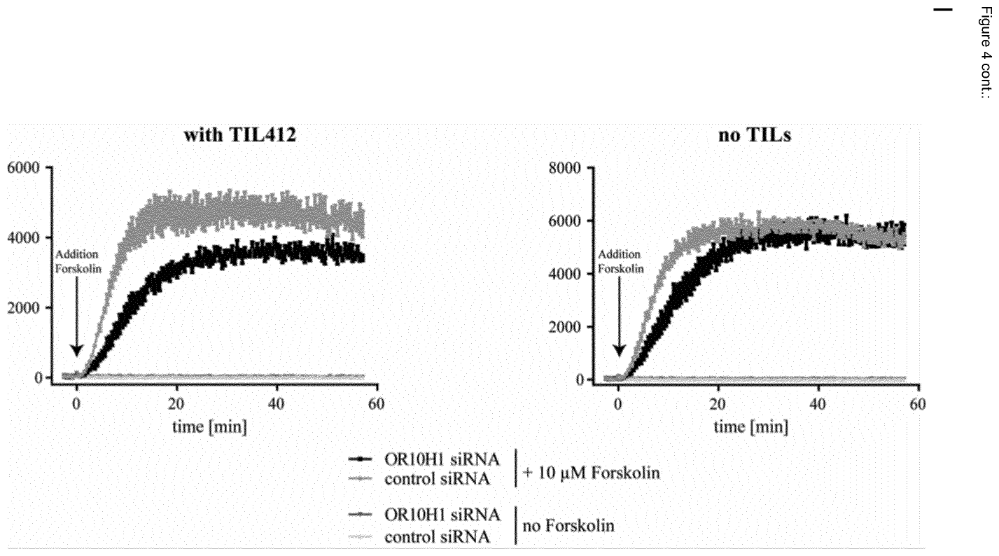

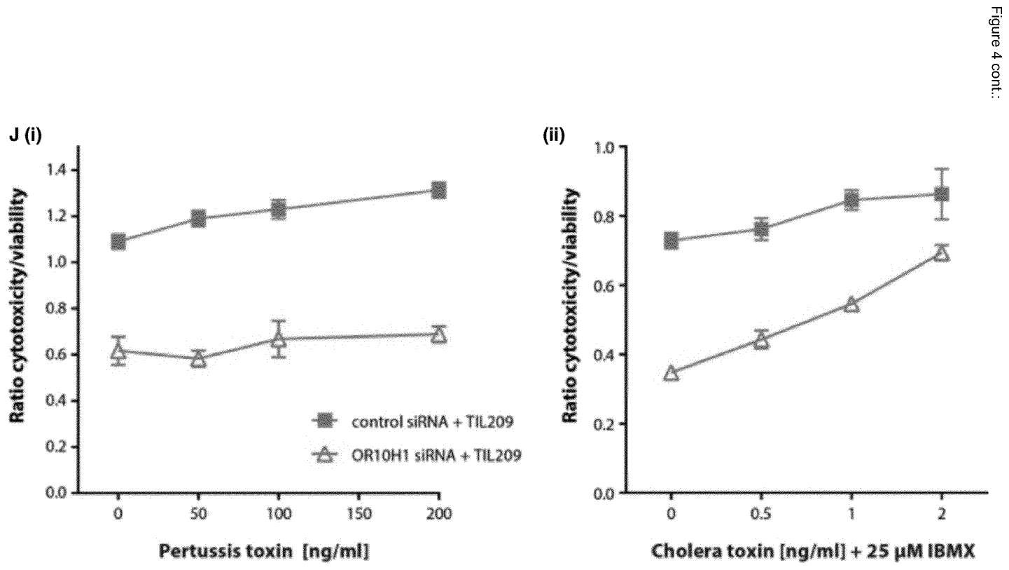

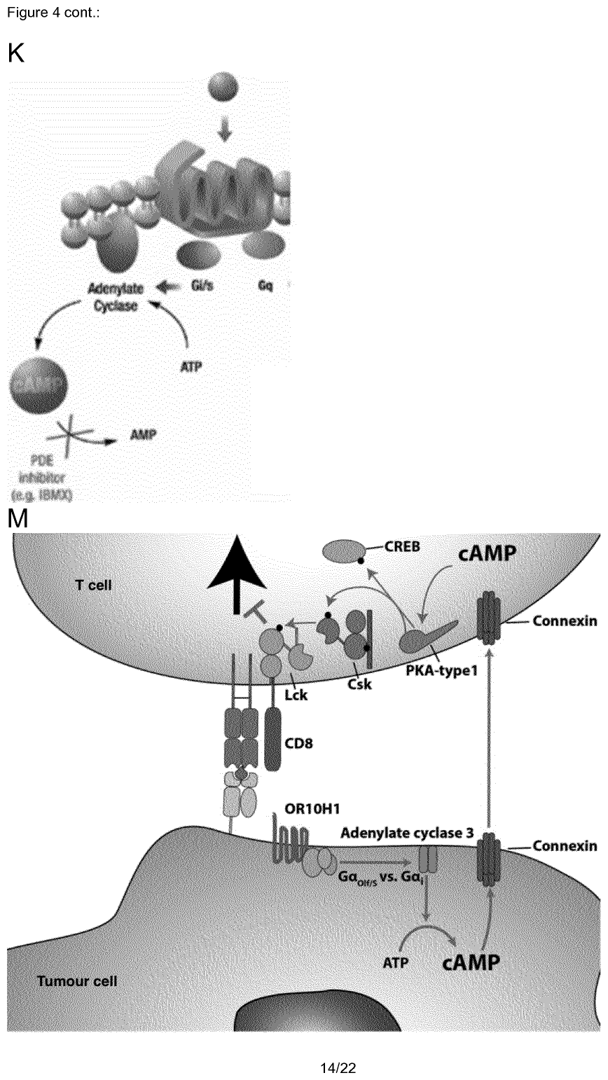

### Example 4: OR10H1 Inhibits TIL Functionality Utilizing cAMP (FIG. 4)

TIL412 were co-cultured with (OR10H1 knock-down or negative siRNA control) M579-A2 for 10 h cells to activate signaling pathways in the TILs, and then the melanoma cells were then removed from the co-culture using magnetic melanoma beads specific for MCSP (Miltenyi Biotech). The purity of the remaining TILs was around 99.5% (not shown). Differential gene expression between the TILs isolated from the OR10H1 knock-down and the negative siRNA control was measured by RNA-Sequencing according to standard procedures. Several genes were significantly up or down regulated (fold change above 0.5 or below −0.5 respectively) in the setting with OR10H1 knock-down M579-A2 (siRNA 1) compared to the negative siRNA control (FIG. 4A). Down-regulated in the TILs were the following genes: Early growth response gene 3 (EGr3) is a key negative transcriptional regulator of T cell activation and induces anergy [1-3]. Interferon-gamma receptor chain 2 (IFNGR2) expressed in high levels inhibits T cell proliferation and may induce apoptosis [4, 5]. Nuclear Receptor Subfamily 4, Group A, Member 2 (NR4A2) is associated with T cell exhaustion in chronic viral infection [6] and might be involved in apoptosis [7]. Up-regulated in the TILs were the following genes: Chemokine (C—X—C motif) ligand 13 (CXCL13) promotes T cell recruitment [8] and facilitates the inflammatory response of antigen-experienced T helper cells [9]. Cytotoxic And Regulatory T Cell Molecule (CRTAM) is expressed (upon TCR activation) on activated T cells and its interaction with Necl-2 (on tumors) promotes IFN-gamma secretion by CD8+ T cells [10]. Vav guanine nucleotide exchange factor 3 (VAV3) is involved in TCR signaling via NFAT and SRF activation [11]. FBJ Murine Osteosarcoma Viral Oncogene Homolog (FOS) together with c-Jun builds up activating protein 1 (AP-1), one of three major transcription factors downstream of TCR signaling [12]. V-Myc Avian Myelocytomatosis Viral Oncogene Homolog (c-Myc) is a transcription factor which is involved in proliferation and metabolic reprogramming upon T lymphocyte activation [13, 14]. It modulates the generation of CD8+ immunological memory in tumors and viral infection [15, 16]. Interferon regulatory factor 4 (IRF4) is a transcription factor necessary for the expansion and effector differentiation of CD8+ T cells and represses genes involved in cell cycle arrest and apoptosis [17, 18].

Upon Ingenuity pathway analysis (Qiagen; FIG. 4B) to investigate functions in which the differentially expressed genes are enriched (positive or negative activation) in TILs and which signaling networks play a role, the inventors surprisingly found that of the eight pathways analyzed, only CREB is differentially phosphorylated in the setting with OR10H1-negative melanoma. The log 2 ratio (compared to unstimulated TILs) for CREB is significantly reduced after the knock-down of OR10H1 (p=0.006; FIG. 4C).

Western blot analysis for phospho-CREB and ATF1 (same phosphorylation site) in TIL412 confirmed the reduced activation depending on OR10H1 knock-down on melanoma cells (FIG. 4F). cAMP response element-binding protein (CREB) downstream of TCR activation is widely known to be important for proliferation and survival of T cells [19] but has been associated with T cell anergy as well [20]. Therefore, key factors of TCR signaling (Lck, LAT, CREB, ZAP-70, Syk, CD3e and ERK1/2) were analysed.

Knock-down of OR10H1 on melanoma cells leads to decreased phosphorylation of CREB and Lck (inhibitory phospho-Tyrosin 505) in TILs (FIGS. 4D and 4E). Importantly however, OR10H1 does not play a role in modulating CD3e activation (phosphorylation): CD3e does not show increased phosphorylation levels after co-culture with melanoma cells/TILs (regardless of OR10H1 knock-down) compared to unstimulated TILs, and the same is observed for ERK1/2. Yet, over-stimulation with PMA and ionomycin does dramatically increase phosphorylation of ERK1/2 but does not affect CD3e.

Phosphorylation of Linker of activated T cells (LAT) slightly increases in TILs in the first 30 minutes of co-culture and stays stable up to two hours (for OR10H1-positive and OR10H1-negative melanoma cell/TIL co-cultures and over-activation PMA and ionomycin). Spleen tyrosine kinase (Syk) gets activated in 5 minutes and later on decreases below the level of unstimulated TILs in all three settings. Finally, phosphorylation of Zeta-chain-associated protein kinase 70 (ZAP70) increases in the first 5 minutes and stays stable for 2 h if co-cultured with melanoma cells/TILs (regardless of OR10H1 knock-down). Over-activation with PMA and ionomycin leads to an increased phosphorylation of ZAP70.

Co-culture of TIL412 with OR10H1-positive M579-A2 melanoma cells leads to a dramatically increased phosphorylation of CREB after 2h in TILs (FIG. 4D). Interestingly, in the first 30 min after activation the phosphorylation levels are similar to the OR10H1 knock-down setting. Over activation with PMA and ionomycin leads to a faster onset of CREB phosphorylation but the levels are similar to the control siRNA (OR10H1-positive) setup. In contrast, Lymphocyte-specific protein tyrosine kinase (Lck) becomes strongly dephosphorylated after 30 min in TILs if co-cultured with OR10H-negative M579-A2 melanoma cells or over-activated with PMA and ionomycin but not in the control siRNA (OR10H1-positive) setup (FIG. 4E).

As described above, TCRs of TILs which were co-cultured with melanoma cells were activated in the same way, but showed different signaling at two important hubs, namely CREB and Lck, depending on whether OR10H1 was present or absent on the target melanoma cells. Therefore, OR10H1-mediated inhibition of T cell activity appears to converge here. Lck has two phosphorylation sites but phosphoplex analysis of Lck measures total Lck phosphorylation. Phosphorylation of Lck-Tyr384 stabilizes the active conformation whereas phosphorylation of Lck-Tyr505 promotes the auto-inhibited conformation of Lck [21-23]. Western blots for phospho-Lck confirmed that phosphorylation of the inhibitory Tyr505 residue was strongly reduced after the knock-down of OR10H1 suggesting an increased activation of Lck in TILs. Indeed, inhibition of Lck activity by a small molecule inhibitor is shown to abrogate the OR10H1 knock-down effect on T-cell mediated cytotoxicity (as reflected by the ratio of cytotoxicity/viability) of melanoma cells (FIG. 4G). Protein kinase A (PKA) is activated by cAMP and in turn activates c-Src Tyrosine Kinase (Csk). Csk abrogates Lck activity by phosphorylation of Lck-Tyr505 [24, 25]. Indeed, the inventors were able to confirm by Western blot analysis an enhancement of PKA phosphorylation in TILs after the OR10H1-positive (i.e. control siRNA) M579-A2 melanoma cell/TIL co-culture. (FIG. 4F). Olfactory receptor signaling activates a unique G protein (G-alpha-Olf) and subsequently adenylate cyclase type III (FIG. 4H). RNA-sequencing expression data suggest that both genes are expressed in M579-A2. M579-A2 melanoma cells (OR10H1-positive and OR10H1-negative) were transiently transfected with a cAMP reporter luciferase construct and co-cultured with TIL412. The production of cAMP in melanoma is altered by OR10H1 knock-down only if there is an interaction with TILs, while co-culture of M579-A2 cells with TILs alone was not sufficient to trigger a cAMP response (not shown). Indeed, after co-culture of M579-A2 (OR10H1-positive or OR10H1-negative) with TILs for 2 h, 10 μM forskolin was added to raise cAMP levels: without a preceding co-culture with TILs there is no difference in cAMP response in M579-A2 cells, whereas in the presence of TILs the cAMP response in M579-A2 cells is reduced if OR10H1 is knocked-down on the melanoma cells (FIG. 4I).

The inventors demonstrated that TIL-mediated lysis of M579-A2, after OR10H1 knockdown, can be reversed by cholera toxin (CTX). The impact of pertussis (PTX) and cholera toxin (CTX) on TIL-mediated tumor lysis was measured. Pertussis toxin (derived from Bordetella pertussis) inhibits the inhibitory G protein alpha subunit (G-alpha-I), which is thus prevented from inhibiting adenylate cyclase (AC3) production of cAMP. Cholera toxin on the other hand inhibits the GTPase function of G-alpha-S and of G-alpha-Olf (collectively, designed herein by G-alpha-Olf/S), keeping them in their activated states leading to increased activation of AC3, and accumulation of cAMP by PDE inhibition (FIG. 4K). The addition of cholera but not pertussis toxin abrogated the OR10H1 knockdown-mediated increase in TIL-mediated tumor lysis (FIG. 4J). Here, tumor cells were pretreated with the toxins followed by vigorous washing to prevent any direct effect on the TILs. The pretreatment with pertussis toxin for 6 hours slightly decreased the TIL-mediated tumor lysis in control siRNA transfected M579-A2-luc (FIG. 4J(i). This was not observed in cells transfected with OR10H1 siRNA. The pretreatment with cholera toxin and 25 μM 3-isobutyl-1-methylxanthine (IBMX) leads to a slight decrease in TIL-mediated tumor lysis in the control setup but dramatically decreases killing of OR10H1-negative M579-A2-luc (FIG. 4J(ii)). This decrease in TIL-mediated tumor killing occurs in a cholera toxin concentration-dependent manner. Without cholera toxin pretreatment (but with IBMX) around 65-70% of OR10H1-negative M579-A2-luc cells get lysed in a TIL-dependent manner (20-25% in the control setup). At a concentration of 2 ng/ml, cholera toxin pretreatment decreased TIL-mediated lysis to around 30% in both settings. Hence, at this concentration, the effect of OR10H1 knockdown on TIL-mediated lysis is almost completely abrogated (compared to the negative control). This data suggests a major role for the G-alpha-Olf/S pathway in OR10H1-mediated inhibition of TIL cytotoxicity.

It was surprising to observe data suggesting that TIL-driven cytotoxicity of the melanoma cells may be inhibited by a mechanism involving connexin 32 (Cx32) and the transport of cAMP from the tumour cell to the T cell. We validated by RNA-sequencing that M579-A2 cells express connexin 32, a protein involved in the formation of gap-junctions. Such gap-junctions are selective for the transport of cAMP and cGMP [28], and hence the potential for cAMP transport from M579-A2 to the TIL412. Indeed, preliminary results suggest that blockade of Cx32 leads to an increased killing of melanoma cells by TILs only if OR10H1 is present (not shown). This is consistent with recent studies which have shown that regulatory T cells (Tregs) and tumor cells can transport cAMP via gap-junctions into T cells and lead to inhibition through phosphorylation of Lck and CREB [26, 27].

Overall, the inventors propose the following proposed mode of action for OR10H1-mediated inhibition of CD8+ TIL functionality (FIG. 4M). T cell recognition of tumour cells results in TCR-mediated activation of Lck and subsequent killing of the target cell. Binding of an unknown ligand to the olfactory receptor OR10H1 induces the dissociation of G-alpha-Olf/S from the trimeric G-protein complex. G-alpha-Olf/S activates adenylate cyclase 3 (AC3) increasing cAMP concentration. Whilst not wishing to be bound by this or any theory, cAMP is believed to activate tumour-associated PKA which in turn phosphorylates connexin 32 and increased permeability. cAMP diffuses through connexin 32 gap junctions into CD8+ TILs and activates TIL-associated PKA. PKA phosphorylates/activates Csk and CREB. The transcription factor CREB induces gene expression associated with an exhausted TIL phenotype. Csk phosphorylates Lck at an inhibitory tyrosine residue and thus abrogates TCR-mediated signaling.

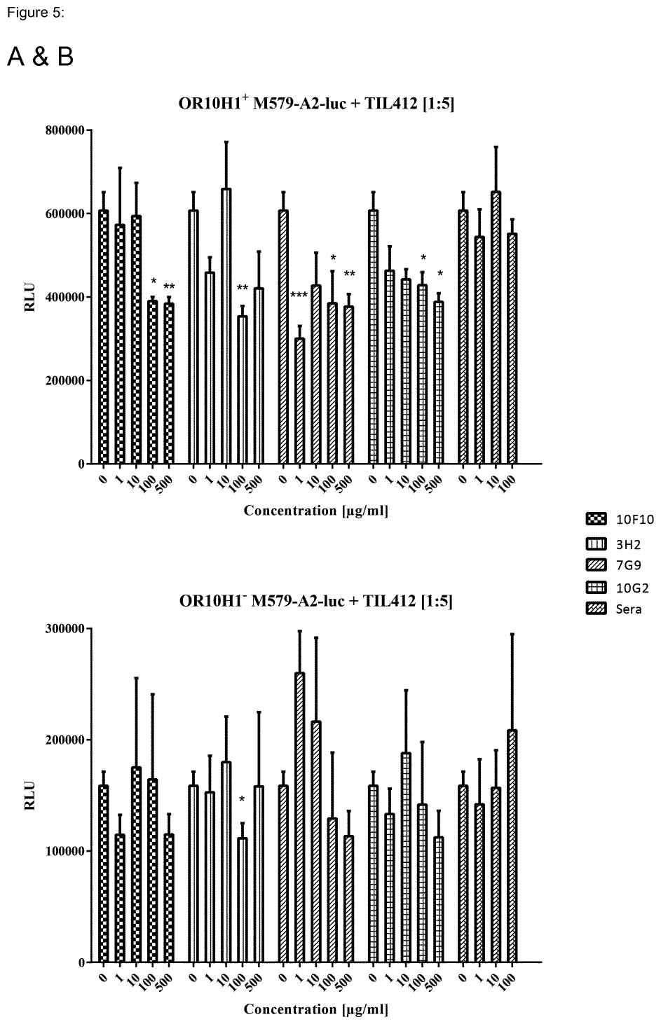

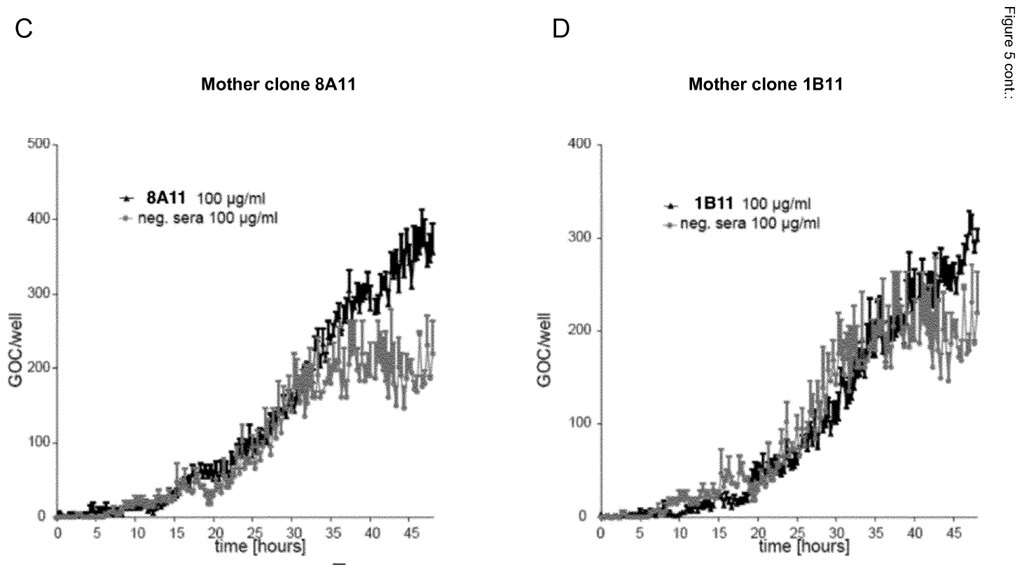

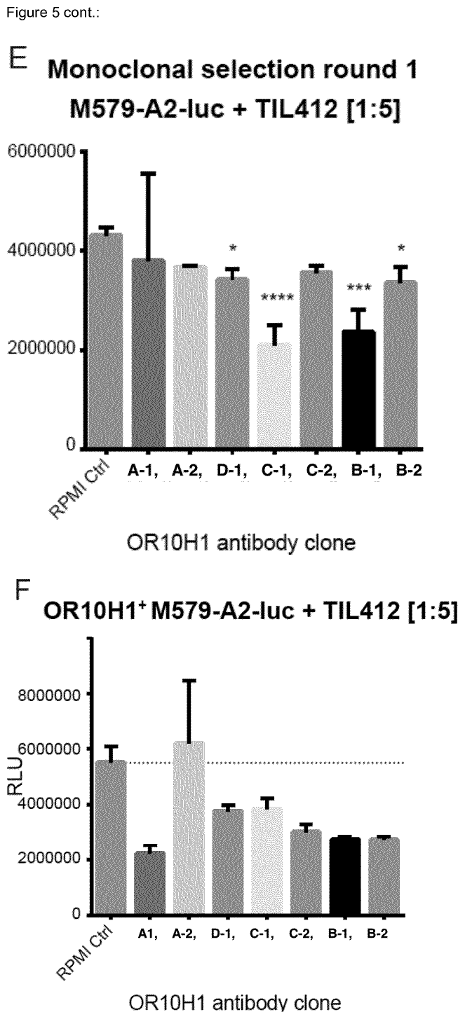

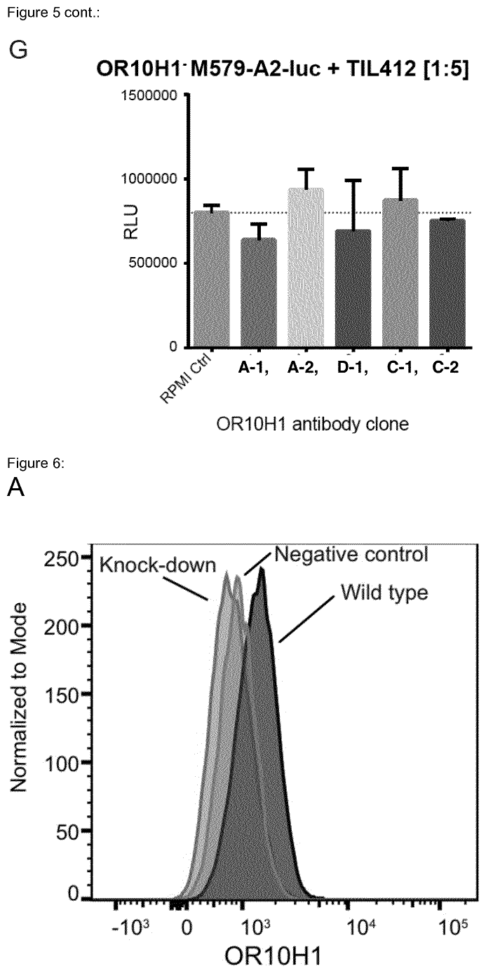

### Example 5: Development of Antibodies Against OR10H1 and Their Functional Activity (FIG. 5)

For generating antibodies against OR10H1, genomic immunization of rats is conducted at Aldevron (Freiburg), described briefly as follows.

Firstly, an OR10H1 construct is cloned into an Aldrevon immunization vector (pB8-OR10H1) and an Aldrevon screening vector (pB1-OR10H1). Transient transfection of these vectors into mammalian cells is used to confirm cell surface expression. Cell surface expression of such proteins—containing a vector-derived N-terminal tag-sequence—is analysed by flow cytometry on non-fixed living cells using an anti-tag murine antibody and a goat anti-mouse IgG fluorescently labeled conjugate as a secondary antibody. Secondly, a number of rats are immunized with the immunization vector (pB8-OR10H1) over 81 days with 7 genetic applications (IS81d-7)). Thirdly, sera of immunized rats are then tested (by flow cytometry) for reactivity against mammalian cells transiently transfected with the screening vector (pB1-OR10H1) using a goat anti-rat IgG fluorescently labeled conjugate as a secondary antibody. Compared to pre-immunization sera, a significant reactivity is observed. Fourthly, splenocytes are isolated from the rats' spleen, the resulting B cells fused with immortalized myeloma cells by standard technologies, and the resulting hybridoma clones screened for the reactivity of their supernatant against the recombinant mammalian cells transiently transfected with the screening vector (as described above). Screening and enrichment of such hybridoma clones will result in a number of mother clones being selected which express antibodies having the desired binding and/or functional effects on OR10H1.

Such screening and enrichment of hybidoma clones resulted in 4 mother clones being selected (1C3, 1B11, 8A11 and 4B4).For example, each of these antibodies produced by such clones can be shown to have a significant impact on TIL-mediated melanoma killing at a concentration of, for example, 100 μg/ml in the OR10H1-positive M579-A1-Luc/TIL412 assay (FIGS. 5A and B) and/or efficacy in the IncuCyte cytotoxicity assay based on caspase activation (eg for 8A11 and 1B11, as in FIGS. 5C and D), in each case as described above.

Next, these mother clones are single-cell sorted to generate individual hybridoma clones that produce monoclonal antibodies, which are then tested for their functional activity in the OR10H1-positive M579-A1-Luc/TIL412 assay as described above, and individual hybridoma clones were selected (based on their significant binding and/or functional effects) for sequencing of the antibody they produce. FIGS. 5E, F and G show the functional activity of monoclonal antibodies produced by individual hybridoma clones in the OR10H1-positive M579-A1-Luc/TIL412 assay as described above.

The isolation of antibodies by the above procedure demonstrates the ability of antibodies of the present invention to have therapeutic and research utility as and/or in the development of compounds for the treatment of proliferative disease such as a cancer disease, in particular OR10H1-positive cancers.

### Example 6: Detection of OR10H1 Using an Antibody of the Present Invention (FIG. 6)

The post-immunisation poly sera (or an antibody produced by one hybridoma clone isolated as described above) is used to specifically detect OR10H1 expressed on the surface of tumor cells. FACS detection of melanoma M579-A2 cells transiently transfected with negative control siRNA or OR10H1 knock-down siRNA1 is conducted (FIG. 6A), using a chicken anti-rat IgG-AlexaFluor 647 conjugate (ThermoFisher A-21472) as a secondary antibody. Cells knocked-down for OR10H1 show reduced fluorescence compared to wild-type (i.e., OR10H1-positive) cells.

Alternatively, tumour cells shown by RT-PCR not to express OR10H1 (e.g. KMM-1 myeloma cells) are not detected by an antibody purified from a hybridoma clone isolated as described above, while those shown by RT-PCR to express OR10H1 (e.g. M579-A2) are shown to be detected at all concentrations (e.g., 10 μg to 100 μg), while control isotype antibody does not distinguish between the two cells types.

In particular, the antibody purified from clone Di-8A11-H12-E6 shows specificity (compared to control IgG2a isotype antibody) by binding to those melanoma cells (M579-A2) that express OR10H1 but not binding to myeloma cells (KMM-1) that do not express OR10H1 (FIG. 6B). OR10H1 expression is determined by RT-PCR. Such results demonstrate the ability of antibodies of the present invention to detect the expression of OR10H1 protein and their utility to determine increased resistance of a cell against an immune response, such as of a cancer cell.

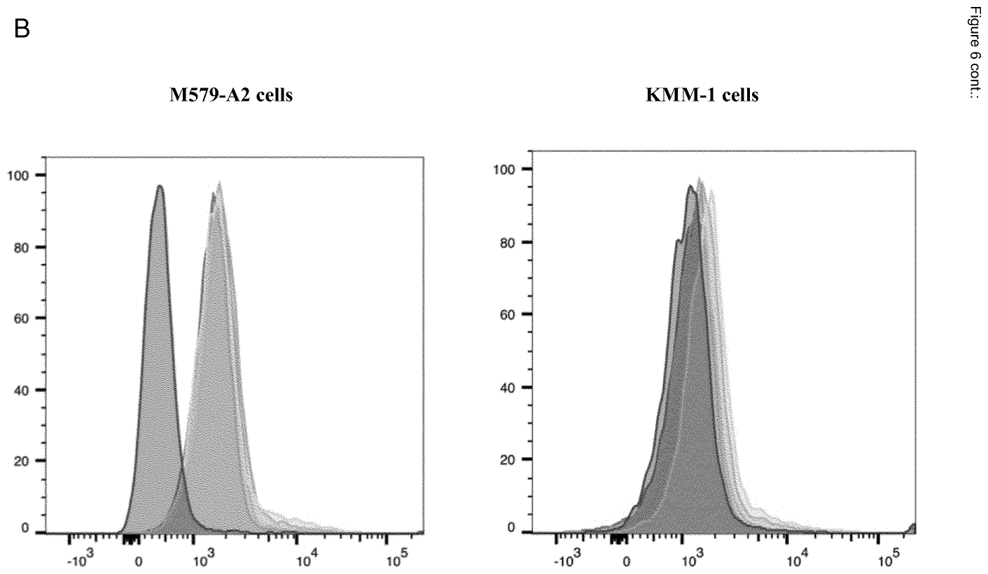

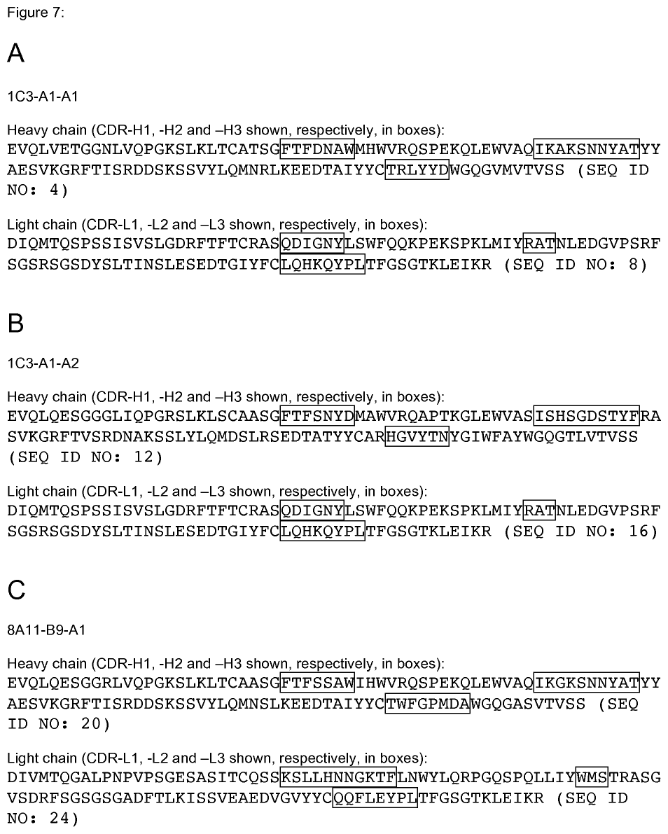

### Example 7: Sequences of Antibodies of the Invention (FIG. 7)

The sequences of variable domains of the antibodies produced by the hybridoma clones of Example 5 are determined by standard procedures at Antibody Designs Laboratories (San Diego). Briefly, the following general procedure is followed: (1) total RNA extraction and cDNA Synthesis; (2) 5′RACE extension; (3) amplification of VH and VL domains including leader sequence and partial constant regions CH1 and CL; (4) cloning of PCR positive reactions; (5) colony PCR and sequencing of clones with proper insert size; and (6) sequencing analysis up to 5× coverage or 10 clones per chain. From the nucleic acid sequence the amino acid sequence of the variable domains of chains of each antibody is determined.

The amino acid sequence of the variable domains of heavy and light chain of exemplary antibodies of the invention is shown in FIGS. 7A, B and C. Hybridoma clone Di-8A11-H12-E6 is deposited at the DSMZ (Leibniz Institut DSMZ-Deutsche Sammlung von Mikroorganismen and Zellkulturen GmbH, Inhoffenstrasse 7B, D-38124 Braunschweig, Germany) in accordance with the Budapest Treaty. (DSMZ Deposition Number: DSM ACC3310, deposited 26 Oct. 2016, Depositor: Deutsches Krebsforschungszentrum (DKFZ) [Stiftung des oeffentlichen Rechts], Im Neuenheimer Feld 280, 69120 Heidelberg, Germany). Hybridoma clone Di-8A11-H12-E6 is a hybridoma comprising Rat B lymphocytes fused with murine Ag8 myeloma cells, and can be cultured at 37oC in a 5% CO2 atmosphere in RPMI-1640 medium supplemented with 10% Fetal calf serum, 10 U/ml human recombinant IL-6 and 1% penicillin-streptomycin (10,000 U/mL). The optimal split ratio is 1:1. The viability of these hybridoma cells may be relatively low when first thawed, however this recovers over a few days in culture. Each antibody chain sequence comprises c-terminally a constant region (Table E3).

Using standard procedures, for example briefly: trypsin digestion of antibody protein followed by peptide mass fingerprinting using MALDI-TOF mass spectrometry (e.g., Toplab GmbH, Martinsried, Germany), the IgG subclass of the constant region of the antibody chain(s) is determined, by comparison of the peptide mass-peaks measured by MALDI-TOF mass spectrometry to the mass of peptide fragments predicted to be obtained from trypsin digestion of the respective rat IgG subclasses (e.g. Table E3).

TABLE E3
Rat Constant Domain Regions:

| Rat IgG | Uniprot ID for constant-region | Constant region |
|---|---|---|
| subclass | (accessed 20 Oct. 2016) | length (aa) |
| IgG1 | P20759 (Sequence version 1 of 1 Feb. 1991; | 326 |
| | Entry version 113; 5 Oct. 2016) | |
| IgG2a | P20760 (Sequence version 1 of 1 Feb. 1991; | 322 |
| | Entry version 116; 5 Oct. 2016) | |
| IgG2b | P20761 (Sequence version 1 of 1 Feb. 1991; | 333 |
| | Entry version 105; 5 Oct. 2016) | |
| IgG2c | P20762 (Sequence version 1 of 1 Feb. 1991; | 329 |
| | Entry version 96; 5 Oct. 2016) | |
| IgG kappa | P01836 (Sequence version 1 of 21 Jul. 1986; | 106 |
| (A allele) | Entry version 80; 5 Oct. 2016) | |
| IgG kappa | P01835 (Sequence version 1 of 21 Jul. 1986; | 106 |
| (B allele) | Entry version 89; 5 Oct. 2016) | |

### Example 8: Epitope Mapping Using Alanine Scan of the Extracellular Portions of OR10H1

A mutant library of OR10H1 is generated, where each residue in the extracellular region of OR10H1 (amino acids 1-25, 76-99, 161-197 and 260-272) is individually substituted by alanine, such that each mutant OR10H1 protein carries only one such alanine substitution as well as an N-terminal tag. Wildtype OR10H1 and each of the mutant OR10H1 variants is individually transiently transfected and expressed on the surface of CHO cells. The expression of wildtype and mutant OR10H1 is confirmed by flow cytometry using anti-tag antibodies. For epitope mapping, anti-OR10H1 antibodies (generated as described above) are diluted to 1-10 μg/ml in PBS/3% FCS and added to the transfected cells. After incubation the cells are washed using PBS/3% FCS. Bound anti-OR10H1 antibody is detected with goat anti-rat PE-conjugate using flow cytometry. The loss of binding of the OR10H1 antibodies to certain alanine mutants indicates the involvement of the corresponding residues in the binding of the respective antibody.

### Example 9: Epitope Mapping Using Domain Swapping

The regions harboring N-terminus or extracellular loops of human OR10H1 (amino acids 1-25, 76-99, 161-197 and 260-272) are replaced by the homologous fragments of human OR10H2, human OR10H5, mouse OLFR55 or cynomolgus OR10H1 using gene synthesis. Each mutant OR10H1 protein carries an N-terminal tag. Wildtype OR10H1 and each of the domain-swapped OR10H1 variants is individually transiently transfected and expressed on the surface of CHO cells. The expression of wildtype and mutant OR10H1 is confirmed by flow cytometry using anti-tag antibodies. For epitope mapping anti-OR10H1 antibodies (generated as described above) are diluted to 1-10 μg/ml in PBS/3% FCS and added to the transfected cells. After incubation the cells are washed using PBS/3% FCS. Bound anti-OR10H1 antibody is detected with goat anti-rat PE secondary antibody using flow cytometry. The loss of binding of the OR10H1 antibodies to certain mutants indicates the involvement of the corresponding domain in the binding of the respective antibody.

### Example 10: Epitope Mapping Using Peptides Representing the Extracellular Portions of OR10H1

Various different peptides are synthesized that represent overlapping sequences, and the entire regions, of the extracellular domains of OR10H1, representing the N terminus (amino acids 1-25) and the first (amino acids 76-99), second (amino acids 161-197) and third (amino acids 260-272) extracellular loops. Each peptide is immobilized on the wells of an ELISA plate, the wells are washed with PBS and blocked with PBS/3% skimmed milk powder. Anti-OR10H1 antibodies (generated as described above) are diluted to 0.5-5 μg/ml in PBS and added to the immobilized peptides. After incubation, the wells are washed with PBS/0.1% BSN 0.05% Tween 20 and then with PBS. Bound antibody is detected with goat anti-rat HRP-conjugate using TMB (3,3′,5,5′-Tetramethylbenzidine) liquid substrate and absorbance is read at 655 nm. Binding of the anti-OR10H1 antibodies indicates mapping to the corresponding extracellular regions of OR10H1.

### Example 11: Screening Compounds to Identify Them as Modulators of OR10H1 Function and/or Activity

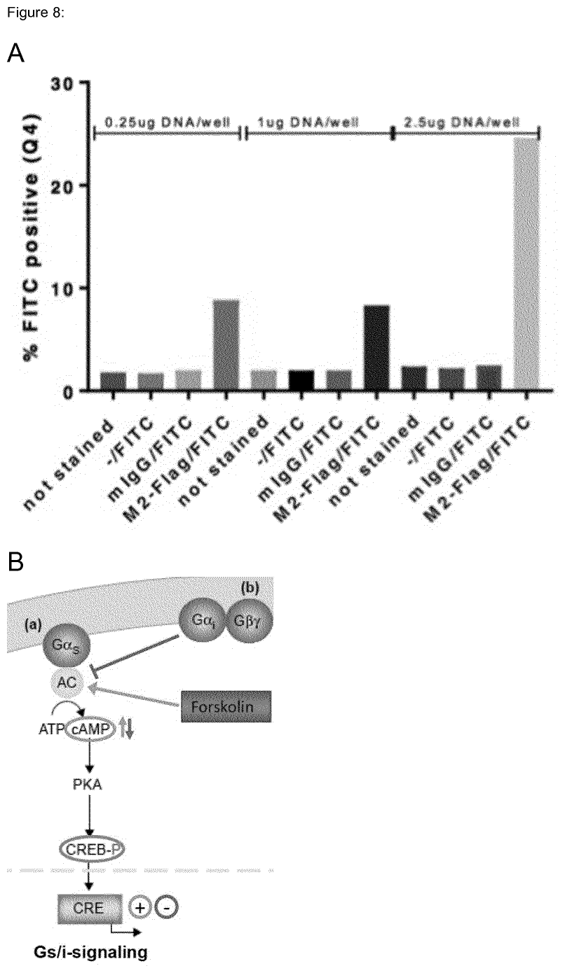

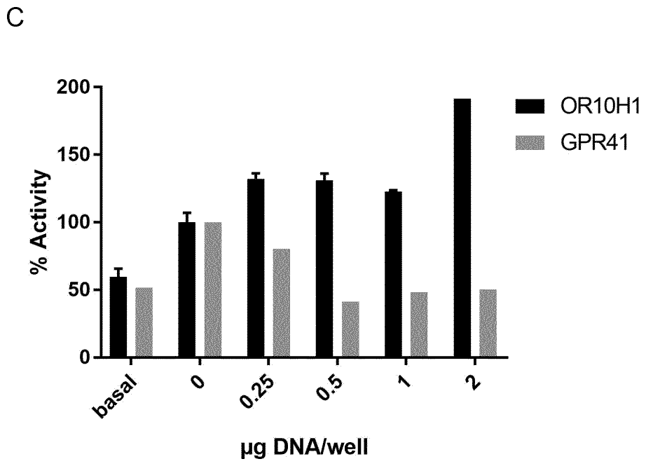

An OR10H1-dependent cAMP response was specifically engineered into a reporter cell line that does not typically express OR10H1. Transient transfection of HEK293 cells containing a cre-Luciferase reporter system (eg, Cignal Lenti CRE Reporter (luc) Kit: CLS-002L, Qiagen, Hildon, Germany; or Assay.Works GmbH, Regensburg, Germany) with DNA constructs encoding N-terminal FLAG-tagged OR10H1 was shown to lead to surface expression of OR10H1 on the surface of HEK cells, as determined by detection of binding of anti-FLAG-tag using a flow cytometer (FIG. 8A). Following 10 uM forskolin stimulation, cAMP production was significantly increased (as measured by cre-luc activity; principle shown in FIG. 8B) in such OR10H1-expressing HEK293 reporter cells. Conversely, HEK293 reporter cells transfected with the G-protein coupled receptor GPR41 which (in contrast to ORH10H1) signals via the G-alpha-I pathway led to decreased cAMP levels as determined by the cre-luciferase activity (FIG. 8C).

A stably transfected version of the above reporter cell line is generated: a HEK293 cell line comprising the cre-Luciferase reporter system and surface-expressing OR10H1. Such a cell line is characterized by a constitutively higher basal activity of the OR10H1 receptor as determined by the increased cAMP levels/cre-luciferase activity compared to the parental cell. Alternatively, co-culture of such a cell line with activated T cells can further elevate cAMP production, as detected by an increase in luciferase signal. Use of this assay system in a high-throughput setting enables the screening of a library of candidate compounds being drug-like small molecules (such as those comprised in the Compound Cloud as obtainable from BioAscent, Newhouse, Scotland) to identify those compounds from the library that reduce the amount of cAMP production in the reporter cell, as detected by a reduction in luciferase signal. Inhibiting/antagonising compounds that so reduce the luciferase signal may reduce the level of the signal reflecting a constitutively activated signal transduction pathway to the basal level, and hence considered “inverse agonists” of the activity of the OR10H1 receptor, or may further reduce the signal to below the basal level and hence be considered inhibitors or antagonists. Such identified small molecules being inhibitors or antagonists (or inverse agonists) of OR10H1, which can be developed for therapeutic uses disclosed herein, such as for the treatment of cancer characterised by aberrant expression and/or activity of a human OR10H1, or a paralogue, orthologue or other variant thereof.

Correspondingly, such an assay may be conducted, eg in the absence of T cells, to identify those compounds that are activators or agonists of OR10H1, by identifying compounds that further increase or elevate the amount of cAMP production in the reporter cell, as detected by an increase in luciferase signal.

Such assay system may also be used to screen, identify and/or characterize the functional effects of one or more ABPs of the invention.

### REFERENCES

Berrien-Elliott M M, Jackson S R, Meyer J M, Rouskey C J, Nguyen T L, Yagita H, Greenberg P D, Dipaolo R J, Teague R M (2013). Durable Adoptive Immunotherapy for Leukemia Produced by Manipulation of Multiple Regulatory Pathways of CD8+ T-Cell Tolerance. Cancer Res 73: 605-616.

Blank C, Brown I, Peterson A C, Spiotto M, Iwai Y, Honjo T, Gajewski T F (2004). PD-L1/B7H-1 inhibits the effector phase of tumor rejection by T cell receptor (TCR) transgenic CD8+ T cells. Cancer Res 64: 1140-1145.

Brahmer J R, Tykodi S S, Chow L Q, Hwu W J, Topalian S L, Hwu P, Drake C G, Camacho L H, Kauh J, Odunsi K et al (2012). Safety and activity of anti-PD-L1 antibody in patients with advanced cancer. New Engl J Med 366: 2455-2465.

Chambers C A, Kuhns M S, Egen J G, Allison J P (2001). CTLA-4-mediated inhibition in regulation of T cell responses: mechanisms and manipulation in tumor immunotherapy. Annu Rev Immunol 19: 565-594

Drake C G, Jaffee E, Pardoll D M. (2006). Mechanisms of immune evasion by tumors. Adv Immunol; 90:51-81.

van Elsas A, Hurwitz A A, Allison J P (1999). Combination immunotherapy of B16 melanoma using anti-cytotoxic T lymphocyte-associated antigen 4 (CTLA-4) and granulocyte/macrophage colony-stimulating factor (GMCSF)-producing vaccines induces rejection of subcutaneous and metastatic tumors accompanied by autoimmune depigmentation. J Exp Med 190: 355-366.

Gao, J., et al, (2013). Advances in the development of cancer immunotherapies. Trends Immunol, 34(2): p. 90-8. 26. Zhu, Y., S. Yao, and L. Chen, Cell surface signaling molecules in the control of immune responses: a tide model. Immunity, 2011. 34(4): p. 466-78.

Hanahan D, Weinberg R A. (2011). Hallmarks of cancer: the next generation. Cell; 144: 646-74.

Khandelwal et al, (2015). High-throughput RNAi screen for detection of immune-checkpoint molecules that mediate tumor resistance to cytotoxic T lymphocytes. EMBO Mol Med 7:450-63

Kipriyanow and Le Gall, (2004). Generation and production of engineered antibodies. Mol Biotechnol., 26(1): 39-60.

Kostelny et al., (1992), Formation of a bi-specific antibody by the use of leucine zippers. J. Immunol. 148:1547-1553.

Rabinovich, G. A., D. Gabrilovich, and E. M. (2007) Sotomayor, Immunosuppressive strategies that are mediated by tumor cells. Annu Rev Immunol., 25: p. 267-96.

Songsivilai and Lachmann, (1990). Bi-specific antibody: a tool for diagnosis and treatment of disease. Clin. Exp. Immunol. 79:315-321;

Topalian S L, Hodi F S, Brahmer J R, Gettinger S N, Smith D C, McDermott D F, Powderly J D, Carvajal R D, Sosman J A, Atkins M B et al (2012). Safety, activity, and immune correlates of anti-PD-1 antibody in cancer. New Engl J Med 366: 2443-2454.

Wang L, Rubinstein R, Lines J L,Wasiuk A, Ahonen C, Guo Y, Lu L F, Gondek D, Wang Y, Fava R A, Fiser A, Almo S, Noelle R J. (2011). VISTA, a novel mouse Ig superfamily ligand that negatively regulates T cell responses. J Exp Med; 208: 577-92.

Woo S R, Turnis M E, Goldberg M V, Bankoti J, Selby M, Nirschl C J, Bettini M L, Gravano D M, Vogel P, Liu C L et al (2012). Immune inhibitory molecules LAG-3 and PD-1 synergistically regulate T-cell function to promote tumoral immune escape. Cancer Res 72: 917-927.

Zitvogel, L., A. Tesniere, and G. Kroemer, (2006). Cancer despite immunosurveillance: immunoselection and immunosubversion. Nat Rev Immunol., 6(10): p. 715-27.
1. Safford, M., et al., Egr-2 and Egr-3 are negative regulators of T cell activation. Nat Immunol, 2005. 6(5): p. 472-480.
2. Collins, S., et al., Opposing regulation of T cell function by Egr-1/NAB2 and Egr-2/Egr-3. European Journal of Immunology, 2008. 38(2): p. 528-536.
3. Zheng, Y., Y. Zha, and T. F. Gajewski, Molecular regulation of T-cell anergy. EMBO Rep., 2008. 9: p. 50-55.
4. Bernabei, P., et al., Interferon-γ receptor 2 expression as the deciding factor in human T, B, and myeloid cell proliferation or death. Journal of Leukocyte Biology, 2001. 70(6): p. 950-960.
5. Schroder, K., et al., Interferon-γ: an overview of signals, mechanisms and functions. Journal of Leukocyte Biology, 2004. 75(2): p. 163-189.
6. Wherry, E. J., et al., Molecular Signature of CD8+ T Cell Exhaustion during Chronic Viral Infection. Immunity, 2007. 27(4): p. 670-684.
7. Zhou, T., et al., Inhibition of Nur77/Nurr1 leads to inefficient clonal deletion of self-reactive T cells. J Exp Med, 1996. 183(4): p. 1879-92.
8. Hui, W., C. Zhao, and S. G. Bourgoin, LPA Promotes T Cell Recruitment through Syn-thesis of CXCL13. 2015. 2015: p. 1-10.
9. Manzo, A., et al., Mature antigen-experienced T helper cells synthesize and secrete the B cell chemoattractant CXCL13 in the inflammatory environment of the rheumatoid joint. Arthritis & Rheumatism, 2008. 58(11): p. 3377-3387.
10. Boles, K. S., et al., The tumor suppressor TSLC1/NECL-2 triggers NK-cell and CD8+ T-cell responses through the cell-surface receptor CRTAM. Blood, 2005. 106(3): p. 779-786.
11. Charvet, C., et al., Membrane localization and function of Vav3 in T cells depend on its association with the adapter SLP-76. Journal of Biological Chemistry, 2005. 280(15): p. 15289-15299.
12. Huang, Y. and R. L. Wange, T Cell Receptor Signaling: Beyond Complex Complexes. Journal of Biological Chemistry, 2004. 279(28): p. 28827-28830.
13. Dose, M., et al., c-Myc mediates pre-TCR-induced proliferation but not developmental progression. Blood, 2006. 108(8): p. 2669-2677.
14. Wang, R., et al., The Transcription Factor Myc Controls Metabolic Reprogramming upon T Lymphocyte Activation. Immunity, 2011. 35(6): p. 871-882.
15. Hague, M., et al., C-Myc regulation by costimulatory signals modulates the generation of CD8+ memory T cells during viral infection. Open Biology, 2016. 6(1).
16. Chandran, S. S., et al., Tumor-Specific Effector CD8+ T Cells That Can Establish Immunological Memory in Humans after Adoptive Transfer Are Marked by Expression of IL7 Receptor and c-myc. Cancer Research, 2015. 75(16): p. 3216-3226.
17. Yao, S., et al., Interferon Regulatory Factor 4 Sustains CD8+ T Cell Expansion and Effector Differentiation. Immunity, 2013. 39(5): p. 833-845.
18. Huber, M. and M. Lohoff, IRF4 at the crossroads of effector T-cell fate decision. European Journal of Immunology, 2014. 44(7): p. 1886-1895.
19. Wen, A. Y., K. M. Sakamoto, and L. S. Miller, The role of the transcription factor CREB in immune function. Journal of immunology (Baltimore, Md.: 1950), 2010. 185(11): p. 6413-6419.
20. Powell, J. D., et al., The −180 Site of the IL-2 Promoter Is the Target of CREB/CREM Binding in T Cell Anergy. The Journal of Immunology, 1999. 163(12): p. 6631-6639.
21. Abraham, N., et al., Enhancement of T-cell responsiveness by the lymphocyte-specific tyrosine protein kinase p561ck. Nature, 1991. 350(6313): p. 62-66.
22. Gervais, F. G., et al., The SH2 domain is required for stable phosphorylation of p561ck at tyrosine 505, the negative regulatory site. Molecular and Cellular Biology, 1993. 13(11): p. 7112-7121.
23. Yamaguchi, H. and W. A. Hendrickson, Structural basis for activation of human lymphocyte kinase Lck upon tyrosine phosphorylation. Nature, 1996. 384(6608): p. 484-489.
24. Vang, T., et al., Activation of the COOH-terminal Src kinase (Csk) by cAMP-dependent protein kinase inhibits signaling through the T cell receptor. The Journal of experimental medicine, 2001. 193(4): p. 497-507.
25. Taskén, K. and a. J. Stokka, The molecular machinery for cAMP-dependent immuno-modulation in T-cells. Biochemical Society transactions, 2006. 34(Pt 4): p. 476-479.
26. Bopp, T., et al., Cyclic adenosine monophosphate is a key component of regulatory T cell-mediated suppression. The Journal of Experimental Medicine, 2007. 204(6): p. 1303-1310.
27. Ye, J., et al., TLR8 signaling enhances tumor immunity by preventing tumor␣induced T␣cell senescence. EMBO Molecular Medicine, 2014. 6(10): p. 1294-1311.
28. Bevans, C. G., Isoform Composition of Connexin Channels Determines Selectivity among Second Messengers and Uncharged Molecules. Journal of Biological Chemistry, 1998. 273(5): p. 2808-2816.

Those skilled in the art will recognize, or be able to ascertain using no more than routine experimentation, numerous equivalents to the specific embodiments described herein both in the Examples and in the body of the entire patent description. Such equivalents are considered to be within the scope of this invention and are intended to be encompassed by the following claims.

All publications, patent applications, patents, and other references mentioned herein are incorporated by reference in their entirety. In case of conflict, the present specification, including definitions, will control. In addition, the materials, methods, and examples are illustrative only and not intended to be limiting.

1. An antigen binding protein (ABP) that binds to a human olfactory receptor 10H1 (OR10H1), or a paralogue, orthologue or other variant thereof,
wherein said ABP binds to an epitope displayed by one or more extracellular domain(s) of said OR10H1, paralogue, orthologue or other variant.

2. The ABP of claim 1, wherein the ABP binds to an OR10H1 variant that shares at least 95%, amino acid sequence identity with SEQ ID NO:1, or to an OR10H1 that is identical to SEQ ID NO: 1.

3. (canceled)

4. The ABP of claim 1 which: (i) binds to said OR10H1 or variant with a KD that is less than 1 nM; and/or (ii) binds to a human cell line expressing said OR10H1 or variant with an EC50 of less than 2ug/mL.

5-6. (canceled)

7. The ABP of claim 1, wherein the ABP is a monoclonal antibody or antigen-binding fragment thereof.

8-11. (canceled)

12. The ABP of claim 1, wherein said epitope(s) is/are formed by a stretch of amino acids of between about 3 and 37 amino acids comprised in any of the amino acid sequences independently selected from the group consisting of: SEQ ID NO:5, SEQ ID NO:6, SEQ ID NO:7, SEQ ID NO:8, SEQ ID NO:9, SEQ ID NO:10 and SEQ ID NO:13.

13. The ABP of claim 12, wherein said epitope(s) is/are formed by a stretch of amino acids comprised in amino acid sequence SEQ ID NO: 5 and/or 9, and wherein said ABP also binds to the cynomolgus orthologue of human OR10H1 (SEQ ID NO: 11).

14-15. (canceled)

16. The ABP of claim 1, which is a modulator of the expression, function, activity and/or stability of said OR10H1 or variant.

17. The ABP of claim 16, wherein said ABP is an inhibitor or antagonist of expression, function, activity and/or stability of said OR10H1 or variant, optionally wherein said ABP: (i) enhances a cell-mediated immune response to a mammalian cell expressing said OR10H1 or variant and/or (ii) increases immune cell activity and/or survival in the presence of a mammalian cell expressing said OR10H1 or variant.

18-24. (canceled)

25. The ABP of claim 1, wherein said ABP is labelled.

26. (canceled)

27. A nucleic acid construct (NAC) comprising a nucleic acid encoding an ABP of claim 1 or a component of said ABP and one or more additional features permitting the expression of the encoded ABP or component of said ABP in a cell.

28-29. (canceled)

30. A pharmaceutical composition comprising (a) at least one ABP of claim 1; (b) at least one NAC comprising a nucleic acid encoding said ABP or component of said ABP; and one or more additional features permitting the expression of the encoded ABP or component of said ABP in a cell; and/or (c) a cell comprising the NAC, wherein the cell is capable of expressing the encoded ABP or component of said ABP, and a pharmaceutically acceptable excipient or carrier.

31. The pharmaceutical composition of claim 30, wherein the pharmaceutical composition is in unit dose form of: (i) between about 10 mg and about 1000 mg for said ABP; and/or (ii) between about 5 μg and about 5000 μg for said NAC; and/or (iii) between about 1×10\hat{ }3 and about 1×10\hat{ }9 cells of said cells.

32. (canceled)

33. A method of enhancing a cell-mediated immune response to a mammalian cell that expresses a human OR10H1 or paralogue, orthologue or other variant thereof, comprising contacting said cell with an ABP of claim 16, or a NAC comprising a nucleic acid encoding said ABP or component of said ABP and one or more additional features permitting the expression of the encoded ABP or component of said ABP in a cell in the presence of an immune cell
wherein said ABP is an inhibitor of the expression, function, activity and/or stability of said OR10H1 or variant, thereby enhancing a cell-mediated immune response.

34. (canceled)

35. A method of treating or preventing a disease, disorder or condition in a mammalian subject in need thereof, comprising administering to said subject at least once an effective amount of the ABP of claim 1, a NAC comprising a nucleic acid construct (NAC) encoding said ABP or component of said ABP and one or more additional features permitting the expression of the encoded ABP or component of said ABP in a cell, a cell comprising the NAC, wherein the cell is capable of expressing the encoded ABP or component of said ABP, or [[the]] a pharmaceutical composition comprising said ABP, said NAC or the cell comprising the NAC, and a pharmaceutically acceptable excipient or carrier.

36. The method of claim 35, wherein (i) an effective amount of said ABP is between about 0.1 mg/kg and about 10 mg/kg per administration; (ii) an effective amount of said NAC is between about 0.05 μg/kg and about 500 μg/kg per administration; or (iii) an effective amount of the cell comprising the NAC is between about 10 cells/kg and about 1×10\hat{ }7 cells/kg.

37. The method of claim 35, wherein said disease, disorder or condition is a proliferative disorder, and/or an infection and/or an immune disorder.

38-47. (canceled)

48. A method of detecting a human OR10H1 or a paralogue, orthologue or other variant thereof in a sample, comprising:
contacting the sample with an ABP of claim 1 and
detecting binding between said ABP and said human OR10H1, paralogue, orthologue or other variant.

49. A method of determining whether a mammalian subject has or is at risk of developing a phenotype associated with cellular resistance against a cell-mediated immune response, said method comprising:
(a) the steps of:
(i) providing a biological sample from said subject, said sample comprising cells or tissue of said subject, or an extract of said cells or tissue; and
(ii) practicing the method of claim 48 with said sample;
or
(b) practicing the method of claim 48 on a biological sample from said subject, said sample comprising cells or tissue of said subject, or an extract of said cells or tissue;
wherein the detection of said human OR10H1 or paralogue, orthologue or other variant thereof in said sample indicates a phenotype associated with cellular resistance against the cell-mediated immune response in said subject.

50. The method of claim 49, wherein said biological sample is a tissue sample from said subject.

51. The method of claim 49, wherein a detected amount of said human OR10H1 or variant thereof greater than a standard or cut-off value indicates a phenotype associated with cellular resistance against the cell-mediated immune response in said subject.

52. (canceled)

53. A kit for detecting human OR10H1 or a paralogue, orthologue or other variant thereof in a biological sample comprising:
(i) at least one ABP of claim 1 and
(ii) optionally, instructions for, and/or one or more additional components suitable for, detecting the binding between said ABP and said human OR10H1 or said paralogue, orthologue or other variant thereof.

54-58. (canceled)

59. A method for identifying or characterizing a compound suitable for the treatment of a disease characterized by expression of OR10H1 or a paralogue, orthologue or other variant thereof, the method comprising the steps of:
(a) providing a first cell expressing a protein of OR10H1 or of a paralogue, orthologue or other variant thereof on the surface of the first cell, and
(b) providing a candidate compound, and
(c) optionally, providing a second cell, wherein the second cell is a cytotoxic immune cell, capable of immunologically recognizing said first cell, and
(d) bringing into contact the first cell and the candidate compound, and optionally the second cell, and
(e) determining subsequent to step (d), the activity of said OR10H1, paralogue, orthologue or other variant thereof of the first cell,
wherein, a reduced or increased activity of said OR10H1 paralogue, orthologue or other variant thereof of the first cell contacted with the candidate compound indicates that the candidate compound is a compound suitable for the treatment of a disease characterized by expression of OR10H1 or a paralogue, orthologue or other variant thereof.

60. The method of claim 59, wherein the activity of said OR10H1 paralogue, orthologue or other variant thereof of the first cell is determined from the amount of cAMP produced by the first cell.

61-62. (canceled)
